(19) United States
(12) Patent Application Publication
(10) Pub. No.: US 2007/0172449 A1
(43) Pub. Date: Jul. 26, 2007

(54) TNF-alpha VARIANT FORMULATIONS FOR THE TREATMENT OF TNF-alpha RELATED DISORDERS

(75) Inventors: David F. Carmichael, Monrovia, CA (US); John R. Desjarlais, Pasadena, CA (US); David E. Szymkowski, Monrovia, CA (US); Jonathan Zalevsky, Riverside, CA (US)

Correspondence Address: MORGAN, LEWIS & BOCKIUS, LLP, ONE MARKET SPEAR STREET TOWER, SAN FRANCISCO, CA, 94105, US

(73) Assignee: Xencor, Inc., Monrovia, CA (US)

(21) Appl. No.: 11/559,379

(22) Filed: Nov. 13, 2006

Related U.S. Application Data
(63) Continuation-in-part of application No. 11/108,001, filed on Apr. 14, 2005.
Application No. 11/108,001 is a continuation-in-part of application No. 10/963,994, filed on Oct. 12, 2004.
Application No. 10/963,994 is a continuation-in-part of application No. 10/262,630, filed on Sep. 30, 2002.
Application No. 10/262,630 is a continuation-in-part of application No. 09/981,289, filed on Oct. 15, 2001, now Pat. No. 7,101,974.
Application No. 09/981,289 is a continuation-in-part of application No. 09/945,150, filed on Aug. 31, 2001, now abandoned.
Application No. 09/945,150 is a continuation-in-part of application No. 09/798,789, filed on Mar. 2, 2001, now Pat. No. 7,056,695.
(60) Provisional application No. 60/186,427, filed on Mar. 2, 2000.
(60) Provisional application No. 60/735,430, filed on Nov. 10, 2005.

(51) Int. Cl. A61K 38/19; A61K 38/13; A61K 31/59; A61K 31/573; A61K 31/525; A61K 31/015

(52) U.S. Cl. 424/85.1; 514/171; 514/167; 514/765; 514/11; 514/251

(57) ABSTRACT

Combination therapies comprising novel TNF-α proteins for the treatment of TNF-α related disorders are provided herein.

This application is a continuation-in-part of U.S. Ser. No. 11/108,001, filed Apr. 14, 2005, which is a continuation-in-part of U.S. Ser. No. 10/963,994, filed Oct. 12, 2004; which is a continuation-in-part of U.S. Ser. No. 10/262,630, filed Sep. 30, 2002, which is a is a continuation-in-part of U.S. Ser. No. 09/981,289, filed Oct. 15, 2001; which is a continuation-part of U.S. Ser. No. 09/945,150, filed Aug. 31, 2001; which is a continuation-in-part of U.S. Ser. No. 09/798,789, filed Mar. 2, 2001, U.S. Ser. No. 09/798,789 claims the benefit of U.S. Provisional Application No. 60/186,427, filed Mar. 2, 2000; and this application further claims benefit of U.S. Provisional Application No. 60/735,430, filed Nov. 10, 2005. All the above identified patent applications are incorporated herein by reference in their entirety.

### FIELD OF THE INVENTION

The invention relates to novel proteins with TNF-α antagonist activity in the treatment of TNF-α related disorders.

### BACKGROUND OF THE INVENTION

Tumor necrosis factor a (TNF-α or TNF-alpha) is a pleiotropic cytokine that is primarily produced by activated macrophages and lymphocytes; but is also expressed in endothelial cells and other cell types. TNF-α is a major mediator of inflammatory, immunological, and pathophysiological reactions. (Grell, M., et al., (1995) Cell, 83:793-802), incorporated by reference. Two distinct forms of TNF exist, a 26 kDa membrane expressed form and the soluble 17 kDa cytokine which is derived from proteolytic cleavage of the 26 kDa form. The soluble TNF polypeptide is 157 amino acids long and is the primary biologically active molecule.

TNF-α exerts its biological effects through interaction with high-affinity cell surface receptors. Two distinct membrane TNF-α receptors have been cloned and characterized. These are a 55 kDa species, designated p55 TNF-R and a 75 kDa species designated p75 TNF-R (Corcoran. A. E., et al., (1994) Eur. J. Biochem., 223:831-840), incorporated by reference. The two TNF receptors exhibit 28% similarity at the amino acid level. This is confined to the extracellular domain and consists of four repeating cysteine-rich motifs, each of approximately 40 amino acids. Each motif contains four to six cysteines in conserved positions. Dayhoff analysis shows the greatest intersubunit similarity among the first three repeats in each receptor. This characteristic structure is shared with a number of other receptors and cell surface molecules, which comprise the TNF-R/nerve growth factor receptor superfamily. TNF signaling is initiated by receptor clustering, either by the trivalent ligand TNF or by cross-linking monoclonal antibodies (Vandevoorde, V., et al., (1997) J. Cell Biol., 137:1627-1638), incorporated by reference.

Crystallographic studies of TNF and the structurally related cytokine, lymphotoxin (LT) have shown that both cytokines exist as homotrimers, with subunits packed edge to edge in a threefold symmetry. Structurally, neither TNF or LT reflect the repeating pattern of the their receptors. Each monomer is cone shaped and contains two hydrophilic loops on opposite sides of the base of the cone. Recent crystal structure determination of a p55 soluble TNF-R/LT complex has confirmed the hypothesis that loops from adjacent monomers join together to form a groove between monomers and that TNF-R binds in these grooves. Random mutagenesis has been used to identify active sites in TNF-α responsible for the loss of cytotoxic activity (Van Ostade, X., et al., (1991) EMBO J., 10:827836), incorporated by reference. Human TNF muteins having higher binding affinity for human p75TNF receptor than for human p55-TNF receptor have also been disclosed (U.S. Pat. No. 5,597,899 and Loetscher et al., J. Biol. Chem., 268(35) pp263050-26357 (1993)), incorporated by reference.

The different activities of soluble TNF (solTNF) and transmembrane TNG (tmTNF), mediated through discrete interactions with receptors TNFR1 and TNFR2, may account for contrasting beneficial and harmful roles reported for TNF in animal models and in human disease (Kollias, D. Kontoyiannis, Cytokine Growth Factor Rev. 13, 315 (2002); M. Grell et al., Cell 83, 793 (1995); M. Grell, H. Wajant, G. Zimmermann, P. Scheurich, Proc. Natl. Acad. Sci. U.S.A. 95, 570 (1998); C. O. Jacob, Immunol. Today 13, 122 (1992); R. N. Saha, K. Pahan, J. Neurochem. 86, 1057 (2003); and, M. H. Holtmann, M. F. Neurath, Curr. Mol. Med. 4, 439 (2004), all incorporated by reference). For example, paracrine signaling by solTNF is associated with chronic inflammation, while juxtacrine signaling by tmTNF plays an essential role in resolving inflammation and maintaining immunity to pathogens (Holtmann & Neurath, supra; S. R. Ruuls et al., Immunity 15, 533 (2001); M. Canault et al., Atherosclerosis 172, 211 (2004); C. Mueller et al., J. Biol. Chem. 274, 38112 (1999); M. L. 011eros et al., J. Immunol. 168, 3394 (2002); and, M. Pasparakis, L. Alexopoulou, V. Episkopou, G. Kollias, J. Exp. Med. 184, 1397 (1996), all incorporated by reference.) Excess soluble TNF levels are associated with numerous inflammatory and autoimmune diseases, and inactivation of TNF by injectable protein inhibitors reduces symptoms and blocks disease progression (B. B. Aggarwal, A. Samanta, M. Feldmann, in Cytokine Reference J. J. Oppenheim, M. Feldmann, Eds. (Academic Press, London, 2000) pp. 413-434, incorporated by reference). The three FDA-approved TNF inhibitors include a TNFR2-IgG1 Fc decoy receptor (etanercept) and two neutralizing monoclonal antibodies, Remicade® (infliximab) and Humira® (adalimumab). Although effective anti-inflammatory agents, these immunosuppressive drugs can exacerbate demyelinating disease, induce lymphoma, reactivate latent tuberculosis, and increase the risk of sepsis and other infections (as indicated in their warning labels) (N. Scheinfeld, J. Dermatolog. Treat. 15, 280 (2004), incorporated by reference.) A possible explanation for the increased risk of infection comes from studies using TNF knockout and tmTNF knock-in mice, which demonstrate that tmTNF signaling is sufficient to maintain immunity to listerial and mycobacterial infection. In contrast, solTNF is a primary driver of inflammation. Decoy receptors and antibodies can bind to tmTNF, and that etanercept, infliximab, and adalimumab inhibit tmTNF in addition to solTNF (J. Gerspach et al., Microsc. Res. Tech. 50, 243 (2000); H. Mitoma, T. Horiuchi, H. Tsukamoto, Gastroenterology 126, 934 (2004); J. Agnholt, J. F. Dahlerup, K. Kaltoft, Cytokine 23, 76 (2003); B. Scallon et al., J. Pharmacol. Exp. Ther. 301, 418 (2002); C. Shen et al., Aliment. Pharmacol. Ther. 21, 251 (2005); and, H. Mitoma et al., Gastroenterology 128, 376 (2005), all incorporated by reference.) In view of the serious side effects of existing therapies, a therapeutic that is more potent and has a reduced side effect profile is still needed. The present invention shows that an anti-inflammatory agent that inhibits solTNF but spares tmTNF-mediated signaling will block inflammation yet preserve normal immunity to infectious agents.

### SUMMARY OF THE INVENTION

In one aspect, the present invention is directed to a composition for treating a TNF-α related disorder. The composition comprises a therapeutic agent and a variant human TNF-α homotrimer. The homotrimer is comprised of three non-naturally occurring variant TNF-α proteins (e.g. proteins not found in nature) comprising amino acid sequences with at least one amino acid change compared to the wild type TNF-α proteins. Included within the compositions are therapeutic agents that can be administered in combination with the TNF-alpha proteins of the present invention. Therapeutics include therapeutic agents including but not limited to other drugs (e.g. organic molecules or biologics), as well as radiation therapy.

The number of substitutions can be 1, 2, 3, 4 and 5 or more, with at least two being preferred as compared to unmodified human TNF-alpha. Additional substitutions may be made for expression, production, chemical modification reasons.

In certain aspects, the non-naturally occurring variant TNF-α proteins have a substitution selected from positions 21, 23, 30, 31, 32, 33, 34, 35, 57, 65, 66, 67, 69, 75, 84, 86, 87, 91, 97, 101, 111, 112, 115, 140, 143, 144, 145, 146 and 147. In other aspects, the non-naturally occurring variant TNF-α proteins have substitutions selected from the group of substitutions consisting of Q21 C, Q21 R, E23C, N34E, V91E, Q21R, N30D, R31C, R31I, R31D, R31E, R32D, R32E, R32S, A33E, N34E, N34V, A35S, D45C, L57F, L57W, L57Y, K65D, K65E, K651, K65M, K65N, K65Q, K65T, K65S, K65V, K65W, G66K, G66Q, Q67D, Q67K, Q67R, Q67S, Q67W, Q67Y, C69V, L75E, L75K, L75Q, A84V, S86Q, S86R, Y87H, Y87R, V91E, I97R, I97T, C101A, A111R, A111E, K112D, K112E, Y115D, Y115E, Y115F, Y115H, Y1151, Y115K, Y115L, Y115M, Y115N, Y115Q, Y115R, Y115S, Y115T, Y115W, D140K, D140R, D143E, D143K, D143L, D143R, D143N, D143Q, D143R, D143S, F144N, A145D, A145E, A145F, A145H, A145K, A145M, A145N, A145Q, A145R, A145S, A145T, A145Y, E146K, E146L, E146M, E146N, E146R, E146S and S147R.

In another aspect, substitutions may be made either individually or in combination, with any combination being possible. Preferred embodiments utilize at least one, and preferably more, positions in each variant TNF-α protein. For example, substitutions at positions 31, 57, 69, 75, 86, 87, 97, 101, 115, 143, 145, and 146 may be combined to form double variants. In addition triple, quadrupal, quintupal and the like, point variants may be generated.

In a further aspect, the variants comprise polymers, particularly polyethylene glycol (PEG). The polymers, e.g. PEG molecules, can be attached at an amino acid position selected from the group consisting of 10, 21, 23, 24, 25, 27, 31, 42, 44, 45, 46, 86, 87, 88, 90, 107, 108, 128, 110, 140 and 145, with position 31 being particularly preferred. Optionally, the method of attachment is to make a substitution at one or more of these positions to a cysteine, and then chemically attach the polymer molecule to provide specific PEG binding profiles.

In other aspects, the substitution is selected from the group consisting of R31C, C69V, Y87H, C101A, and A145R. In still further aspects, the monomer includes all the substitutions V1M, R31C, C69V, Y87H, C101A, and A145R.

In other aspects, each monomer of the TNF-α homotrimer comprises an amino acid sequence that has at least one amino acid substitution in the Large Domain and at least one amino acid substitution in a domain selected from the group consisting of the DE Loop and the Small Domain as compared to SEQ ID NO:1, wherein said Large Domain substitution is at a position selected from the group consisting of 21, 30, 31, 32, 33, 35, 65, 66, 67, 111, 112, 115, 140, 143, 144, 145 and 146, said Small Domain substitution at a position selected from the group consisting of 75 and 97, said DE Loop substitution at a position selected from the group consisting of 84, 86, 87 and 91, and said monomers are capable of forming TNF-α heterotrimers having at least a 50% decrease in receptor activation as compared to a homotrimer of wild-type TNF-α proteins as determined by a caspase assay.

In certain aspects, the therapeutic agent can be an anti-rheumatoid arthritis agent selected from the group consisting of a non-steroidal anti-inflammation drugs (NSAID), a disease-modifying antip rheumatic drugs (DMARD), and a steroid.

Alternatively, the therapeutic agent is an anti-psoriasis agent selected from the group consisting of anthralin, chrysarobin, corticosteroids, calcipotriene, vitamin D, TazaroteneVitamin A derivatives, methotrexate, cyclosporine, acitretin, alefacept, etanercept, and efalizumab.

In still other aspects, the therapeutic agent is an anti-inflammatory medication.

In still further aspects, methods of treating a TNF-α associated disorder by administering the composition to a patient in need thereof are provided.

### BRIEF DESCRIPTION OF THE DRAWINGS

FIG. 1 depicts the design strategy for TNF-α mutants. FIG. 1A depicts a complex of TNF receptor with wild type TNF-α. FIG. 1B depicts a mixed trimer of mutant TNF-α (TNF-X) and wild type TNF-α. Dark circles are receptor molecules, light pentagons are wild type TNF-α and the dark pentagon is a mutant TNF-α.

FIG. 2 depicts the structure of the wild type TNF-TNF-R trimer complex.

FIG. 3 depicts the structure of the p55 TNF-R extra-cellular domain. The darker appearing regions represent residues required for contact with TNF-α.

FIG. 4 depicts the binding sites on TNF-α that are involved in binding the TNF-R.

FIG. 5 depicts the TNF-α trimer interface.

FIG. 6A depicts the nucleotide sequence of the histidine tagged wild type TNF-α molecule used as a template molecule from which the mutants were generated. The additional 6 histidines, located between the start codon and the first amino acid are underlined. (SEQ ID NO: 1)

FIG. 6B depicts the amino acid sequence of wild type TNF-α with an additional 6 histidines (underlined) between the start codon and the first amino acid. Amino acids changed in the TNF-α mutants are shown in bold. (SEQ ID NO: 2)

FIG. 7 depicts the position and the amino acid changes in the TNF-α mutants.

FIG. 8 depicts the results from a TNF-α activity assay. Only one of the 11 TNF-α variants tested, E146K, was found to have agonistic activity similar to wild-type TNF-α.

FIG. 9 depicts the antagonist activities of the TNF-α variants. The results shown are raw data that have not been normalized as a percent of the control. In this experiment, wild type TNF-α was used at 10 ng/mL. The concentration of the variant TNF-α proteins ranged from 1 ng/mL to 50 μg/mL.

FIGS. 10A and 10B depicts the antagonist activities of the TNF-α variants normalized for percent apoptosis of the control.

FIG. 11 depicts another example of the mutation pattern of TNF-α protein sequences. The probability table shows only the amino acid residues of positions 21, 30, 31, 32, 33, 35, 65, 66, 67, 111, 112, 115, 140, 143, 144, 145, 146 and 147. The occurrence of each amino acid residue at a given position is indicated as a relative probability. For example, at position 21, the wild type amino acid is glutamine; in the TNF-α variants, arginine is the preferred amino acid at this position.

FIGS. 12A-F depicts trimerization domains from TRAF proteins. (SEQ ID NOS: 3-8)

FIG. 13 depicts the synthesis of a full-length gene and all possible mutations by PCR. Overlapping oligonucleotides corresponding to the full-length gene (black bar, Step 1) and comprising one or more desired mutations are synthesized, heated and annealed. Addition of DNA polymerase to the annealed oligonucleotides results in the 5′ to 3′ synthesis of DNA (Step 2) to produce longer DNA fragments (Step 3). Repeated cycles of heating, annealing, and DNA synthesis (Step 4) result in the production of longer DNA, including some full-length molecules. These can be selected by a second round of PCR using primers (indicated by arrows) corresponding to the end of the full-length gene (Step 5).

FIG. 14 depicts a preferred method for synthesizing a library of the variant TNF-α proteins of the invention using the wild-type gene.

FIG. 15 depicts another method for generating proteins of the present invention which uses an overlapping extension method. At the top of FIG. 15A is the template DNA showing the locations of the regions to be mutated (black boxes) and the binding sites of the relevant primers (arrows). The primers R1 and R2 represent a pool of primers, each containing a different mutation; as described herein, this may be done using different ratios of primers if desired. The variant position is flanked by regions of homology sufficient to get hybridization. In this example, three separate PCR reactions are done for step 1. The first reaction contains the template plus oligos Fl and R1. The second reaction contains template plus F2 and R2, and the third contains the template and F3 and R3. The reaction products are shown. In Step 2, the products from Step 1 tube 1 and Step 1 tube 2 are taken. After purification away from the primers, these are added to a fresh PCR reaction together with F1 and R4. During the denaturation phase of the PCR, the overlapping regions anneal and the second strand is synthesized. The product is then amplified by the outside primers. In Step 3, the purified product from Step 2 is used in a third PCR reaction, together with the product of Step 1, tube 3 and the primers F1 and R3. The final product corresponds to the full-length gene and contains the required mutations.

FIG. 16 depicts a ligation of PCR reaction products to synthesize the libraries of the invention. In this technique, the primers also contain an endonuclease restriction site (RE), either blunt, 5′ overhanging or 3′ overhanging. We set up three separate PCR reactions for Step 1. The first reaction contains the template plus oligos Fl and R1. The second reaction contains the template plus F2 and R2, and the third contains the template and F3 and R3. The reaction products are shown. In Step 2, the products of step 1 are purified and then digested with the appropriate restriction endonuclease. The digestion products from Step 2, tube 1 and Step 2, tube 2 and ligate them together with DNA ligase (step 3). The products are then amplified in Step 4 using primer F1 and R4. The whole process is then repeated by digesting the amplified products, ligating them to the digested products of Step 2, tube 3, and amplifying the final product by primers F1 and R3. It would also be possible to ligate all three PCR products from Step 1 together in one reaction, providing the two restriction sites (RET and RE2) were different.

FIG. 17 depicts blunt end ligation of PCR products. In this technique, the primers such as Fl and R1 do not overlap, but they abut. Again three separate PCR reactions are performed. The products from tube 1 and tube 2 are ligated, and then amplified with outside primers F1 and R4. This product is then ligated with the product from Step 1, tube 3. The final products are then amplified with primers F1 and R3.

FIG. 18 is a graphical illustration of the approach of identifying chemical modification sites of the wild type TNF-α molecule.

FIGS. 19 A-D depict the results of a TNFR1 binding assay of wild type TNF-α and certain variants of the present invention.

FIG. 20. A is a chart showing that the TNF-α variants of the present invention are pre-exchanged with wild type TNF-α to reduce TNF-α induced activation of NFkB in 293T cells. FIG. 20B are photographs of the immuno-localization of NFkB in HeLa cells showing that the exchange of wild type TNF-α with the A145/Y87H TNF-α variant inhibits TNF-α-induced nuclear translocation of NFkB in HeLa cells. FIG. 20C depicts the TNF-α variant A145R/Y87H reduces wild type TNF-α-induced Activation of the NFkB-driven luciferase reporter.

FIG. 21 is a chart showing antagonist activity of TNF-α variants.

FIG. 22A-C are dose response curves of caspase activation by various TNF variants.

FIGS. 23A and B shows that a PEGylated TNF-α variant of the present invention when challenged by a Listeria infection has a reduced infection rate as compared to etanercept in a mouse Listeria infection model.

FIG. 24 shows the efficacy of a TNF-α molecule of the present invention against endogenous muTNF in a mouse DBA/1J mouse CIA model. The graph shows therapeutic treatment with a PEGylated TNF-α molecule of the present invention (5 mg/kg IP qd) has comparable in vivo efficacy as compared to etanercept. The bar above the graph shows the protocol of administration in the study.

FIG. 25 shows in vitro data of soluble TNF-α variant antagonism with no effect on transmembrane TNF-α (tmTNF) antagonism.

FIG. 26 shows the TNF-α molecules of the present invention inhibit only soluble TNF and spare transmembrane TNF (tmTNF) activity.

FIG. 27 shows possible mutations to human TNF-α.

FIG. 28 shows that unlike etanercept, DN-TNF molecules are ligand selective TNF inhibitors that inhibit soluble murine or human (a and c) but not transmembrane (b and d) TNF.

FIG. 29 shows that etanercept and DN-TNF have similar efficacy in a mouse anti-collagen antibody induced arthritis model. The experimental efficacy is determined as a measure of hind paw swelling (a) or clinical score (b). DN-TNF safety was examined using a mouse model of L.monocytogenes infection, although etanercept sensitized the mice to infection (as measured by either spleen (c) or blood CFU (d), the DN-TNF treated mice mounted a normal immune response and fought off the infection.

FIG. 30 shows a similar L.monocytogenes infection study in which death was scored as the endpoint. TNF knockout animals as well at the etanercept treated group perished as a result of the infection, while DN-TNF, vehicle, or transmembrane TNF knockin animals has complete survival.

FIG. 31 shows the rational PEGylation strategy to increase DN-TNF pharmacokinetics

FIG. 32 shows the biochemical results of a PEGylation reaction. The gel on the left shows the uniform PEGylation products, and the site-specific PEG adducts formed after conjugating PEGs of various sizes to DN-TNF. The two gels at right show the efficiency of this reaction both in molarity and kinetics.

FIG. 33 shows the PEGylated DN-TNF has the same bioactivity as the unPEGylated form. The top panel shows a kinetic exchange assay that demonstrates the same exchange kinetics between PEGylated or unPEGylated DN-TNF. The bottom panel shows a native PAGE that depicts the steady-state exchange products formed between native and PEGylated or unPEGylated DN-TNF.

FIG. 34 shows that PEGylated and unPEGylated DN-TNFs have equal efficacy in a potency assay (caspase antagonist assay, right panel). The left panel shows a PK study in rat and the increased half-life of PEGylated DN-TNF

FIG. 35 shows the intravenous and subcutaneous pharmacokinetics following single dose administration of 1125-labelled PEGylated DN-TNF.

FIG. 36 shows the fractional absorption (bioavailability) of 1125-labelled PEGylated DN-TNF molecules conjugated with different sized PEG groups.

FIG. 37 shows repeat dose modeling of subcutaneous administration of PEGylated DN-TNF (10 kD-PEG size).

FIG. 38 shows a native PAGE that depicts the steady-state exchange products formed between native mouse or human TNFs and PEGylated DN-TNF.

FIG. 39 shows a kinetic exchange assay that demonstrates the same exchange kinetics between native human and rat TNFs.

### DETAILED DESCRIPTION OF THE INVENTION

The present invention is directed to formulations comprising molecules, including proteins and nucleic acids, possessing TNF-α antagonist activity. In some embodiments, the variants antagonize the activity of both soluble and transmembrane TNF-α activity, while in other embodiments, the variants selectively inhibit the activity of soluble TNF-α over transmembrane TNF-α activity, and in some embodiments, while substantially maintaining transmembrane TNF-α activity. In addition, methods of treatment of TNF-α related conditions using the formulations and molecules of the present invention are disclosed.

In general, the variant TNF-α proteins outlined herein were generated using the PDA® technology, previously described in U.S. Pat. Nos. 6,188,965; 6,269,312; 6,403,312; 6,708,120; 6,801,861; 6,804,611; 6,792,356; and 6,864,359; W098/47089 and WO 01/40091; U.S. Ser. Nos. 09/782,004; 09/927,790; 10/101,499; 10/218,102; 10/666,311; 10/666,307; 10/888,748; all of which are incorporated by reference. In general, these applications describe a variety of computational modeling systems that allow the generation of extremely stable proteins. In this way, variants of TNF proteins were generated that act as antagonists for wild type TNF-α. Other models for assessing the relative energies of sequences with high precision include Warshel, Computer Modeling of Chemical Reactions in Enzymes and Solutions, Wiley & Sons, New York, (1991), as well as the models identified in U.S. Ser. No. 10/218,102, filed Aug. 12, 2002, all hereby incorporated by reference.

In addition, the TNF-α variants may be modified to include polymers, such as PEG, to allow for altered half-lifes and stabilities within the patient. Preferred methods for identifying suitable sites for either the addition or removal of putative PEGylation sites are found in U.S. Ser. No. 10/820,466; 10/956,352; and U.S. Ser. No. 11/200,444, filed on Aug. 8, 2005, entitled “Methods for Rational PEGylation of Proteins, all hereby incorporated by reference in their entirety for such teachings.

Thus, the present invention is directed to variant TNF-α proteins that are antagonists of wild type TNF-α. By “variant TNF-α” or “TNF-α proteins” is meant TNF-α or TNF-α proteins that differ from the corresponding wild type protein by at least 1 amino acid. Thus, a variant of human TNF-α is compared to SEQ ID NO:.1; a mammalian variant is compared to the corresponding wild-type mammalian TNF-α. As used herein variant TNF-α or TNF-α proteins include TNF-α monomers, dimers or trimers. Included within the definition of “variant TNF-α” are competitive inhibitor TNF-α variants. By “competitive inhibitor TNF-α variants” or “ci TNF-α ” or grammatical equivalents is meant variants that compete with naturally occurring TNF-α protein for binding to the TNF receptor without activating TNF signaling, thereby limiting the ability of naturally occurring TNF-α to bind and activate the TNF receptor. By “inhibits the activity of TNF-α ” and grammatical equivalents is meant at least a 10% reduction in wild-type TNF-α activity relative to homotrimeric variant TNF-α or heterotrimeric variant:wild-type TNF-α (e.g. allelelic variants), more preferably at least a 50% reduction in wild-type TNF-α activity, and even more preferably, at least 90% reduction in wild-type TNF-α activity. As described more fully below, in some cases, there is a selective inhibition of the activity of soluble TNFa versus transmembrane TNF-α, and in some cases, the activity of soluble TNF-α is inhibited while the activity of transmembrane TNF-αis substantially and preferably completely maintained.

By “protein” is meant at least two covalently attached amino acids, which includes proteins, polypeptides, oligopeptides and peptides. The protein may be made up of naturally occurring amino acids and peptide bonds, or synthetic peptidomimetic structures, i.e., “analogs” such as peptoids [see Simon et al., Proc. Natl. Acd. Sci. U.S.A. 89(20:9367-71 (1992), incorporated by reference], generally depending on the method of synthesis. Thus “amino acid”, or “peptide residue”, as used means both naturally occurring and synthetic amino acids. For example, homo-phenylalanine, citrulline, and noreleucine are considered amino acids for the purposes of the invention. “Amino acid” also includes imino acid residues such as proline and hydroxyproline. In addition, any amino acid representing a component of the variant TNF-α proteins can be replaced by the same amino acid but of the opposite chirality. Thus, any amino acid naturally occurring in the L-configuration (which may also be referred to as the R or S, depending upon the structure of the chemical entity) may be replaced with an amino acid of the same chemical structural type, but of the opposite chirality, generally referred to as the D-amino acid but which can additionally be referred to as the R— or the S—, depending upon its composition and chemical configuration. Such derivatives have the property of greatly increased stability, and therefore are advantageous in the formulation of compounds which may have longer in vivo half lives, when administered by oral, intravenous, intramuscular, intraperitoneal, topical, rectal, intraocular, or other routes. In the preferred embodiment, the amino acids are in the S- or L-configuration. If non-naturally occurring side chains are used, non-amino acid substituents may be used, for example to prevent or retard in vivo degradations. Proteins including non-naturally occurring amino acids may be synthesized or in some cases, made recombinantly; see van Hest et al., FEBS Lett 428:(1-2) 68-70 May 22, 1998 and Tang et al., Abstr. Pap Am. Chem. S218:U138-U138 Part 2 Aug. 22, 1999, both of which are incorporated by reference herein.

Aromatic amino acids may be replaced with D- or L-naphylalanine, D- or L-Phenylglycine, D- or L2-thieneylalanine, D- or L-1-, 2-, 3- or 4-pyreneylalanine, D- or L-3-thieneylalanine, D- or L-(2-pyridinyl)-alanine, D- or L-(3-pyridinyl)-alanine, D- or L-(2-pyrazinyl)-alanine, D- or L-(4-isopropyl)phenyl-glycine, D-(trifluoromethyl)-phenylglycine, D-(trifluoromethyl)-phenylalanine, D-pfluorophenylalanine, D- or L-p-biphenylphenylalanine, D- or L-p-methoxybiphenylphenylalanine, D- or L-2-indole(alkyl)-alanines, and D- or L-alkylainines where alkyl may be substituted or unsubstituted methyl, ethyl, propyl, hexyl, butyl, pentyl, isopropyl, iso-butyl, sec-isotyl, iso-pentyl, non-acidic amino acids, of C1-C20. Acidic amino acids may be substituted with non-carboxylate amino acids while maintaining a negative charge, and derivatives or analogs thereof, such as the non-limiting examples of (phosphono)alanine, glycine, leucine, isoleucine, threonine, or serine; or sulfated (e.g., —SO3H) threonine, serine, tyrosine. Other substitutions may include unnatural hydroxylated amino acids which may made by combining “alkyl” with any natural amino acid. The term “alkyl” as used refers to a branched or unbranched saturated hydrocarbon group of 1 to 24 carbon atoms, such as methyl, ethyl, n-propyl, isoptopyl, n-butyl, isobutyl, t-butyl, octyl, decyl, tetradecyl, hexadecyl, eicosyl, tetracisyl and the like. Alkyl includes heteroalkyl, with atoms of nitrogen, oxygen and sulfur. Preferred alkyl groups herein contain 1 to 12 carbon atoms. Basic amino acids may be substituted with alkyl groups at any position of the naturally occurring amino acids lysine, arginine, ornithine, citrulline, or (guanidino)- acetic acid, or other (guanidino)alkyl-acetic acids, where “alkyl” is define as above. Nitrile derivatives (e.g., containing the CN-moiety in place of COOH) may also be substituted for asparagine or glutamine, and methionine sulfoxide may be substituted for methionine. Methods of preparation of such peptide derivatives are well known to one skilled in the art. In addition, any amide linkage in any of the variant TNF-α polypeptides can be replaced by a ketomethylene moiety. Such derivatives are expected to have the property of increased stability to degradation by enzymes, and therefore possess advantages for the formulation of compounds which may have increased in vivo half lives, as administered by oral, intravenous, intramuscular, intraperitoneal, topical, rectal, intraocular, or other routes.

Additional amino acid modifications of amino acids of variant TNF-α polypeptides of to the present invention may include the following: Cysteinyl residues may be reacted with alpha-haloacetates (and corresponding amines), such as 2-chloroacetic acid or chloroacetamide, to give carboxymethyl or carboxyamidomethyl derivatives. Cysteinyl residues may also be derivatized by reaction with compounds such as bromotrifluoroacetone, alpha-bromo-beta-(5-imidozoyl)propionic acid, chloroacetyl phosphate, N-alkylmaleimides, 3-nitro-2-pyridyl disulfide, methyl 2-pyridyl disulfide, pchloromercuribenzoate, 2-chloromercuri-4-nitrophenol, or chloro-7-nitrobenzo-2-oxa-1,3-diazole. Histidyl residues may be derivatized by reaction with compounds such as diethylprocarbonate e.g., at pH 5.5-7.0 because this agent is relatively specific for the histidyl side chain, and para-bromophenacyl bromide may also be used; e.g., where the reaction is preferably performed in 0.1M sodium cacodylate at pH 6.0. Lysinyl and amino terminal residues may be reacted with compounds such as succinic or other carboxylic acid anhydrides. Derivatization with these agents is expected to have the effect of reversing the charge of the lysinyl residues. Other suitable reagents for derivatizing alphaamino-containing residues include compounds such as imidoesters, e.g., as methyl picolinimidate; pyridoxal phosphate; pyridoxal; chloroborohydride; trinitrobenzenesulfonic acid; O-methylisourea; 2,4 pentanedione; and transaminase-catalyzed reaction with glyoxylate.

Arginyl residues may be modified by reaction with one or several conventional reagents, among them phenylglyoxal, 2,3-butanedione, 1,2-cyclohexanedione, and ninhydrin according to known method steps. Derivatization of arginine residues requires that the reaction be performed in alkaline conditions because of the high pKa of the guanidine functional group. Furthermore, these reagents may react with the groups of lysine as well as the arginine epsilon-amino group.

The specific modification of tyrosyl residues per se is well known, such as for introducing spectral labels into tyrosyl residues by reaction with aromatic diazonium compounds or tetranitromethane. Nacetylimidizol and tetranitromethane may be used to form O-acetyl tyrosyl species and 3-nitro derivatives, respectively.

Carboxyl side groups (aspartyl or glutamyl) may be selectively modified by reaction with carbodiimides (R′—N—C—N—R′) such as 1-cyclohexyl-3-(2-morpholinyl- (4-ethyl) carbodiimide or 1-ethyl3-(4-azonia-4,4- dimethylpentyl) carbodiimide. Furthermore aspartyl and glutamyl residues may be converted to asparaginyl and glutaminyl residues by reaction with ammonium ions.

Glutaminyl and asparaginyl residues may be frequently deamidated to the corresponding glutamyl and aspartyl residues. Alternatively, these residues may be deamidated under mildly acidic conditions. Either form of these residues falls within the scope of the present invention.

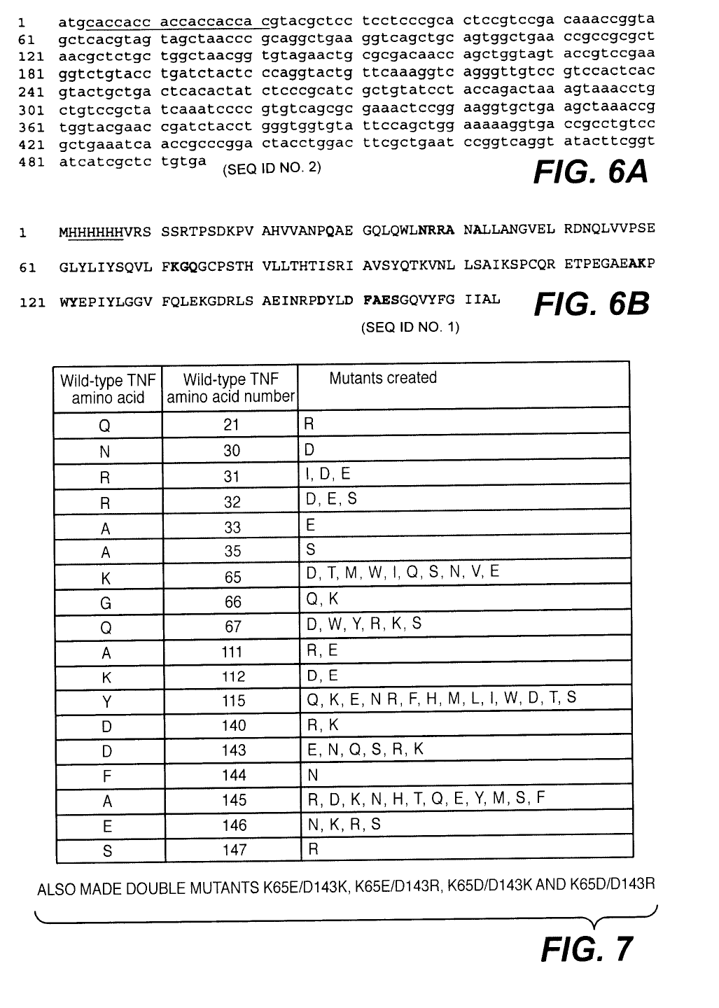

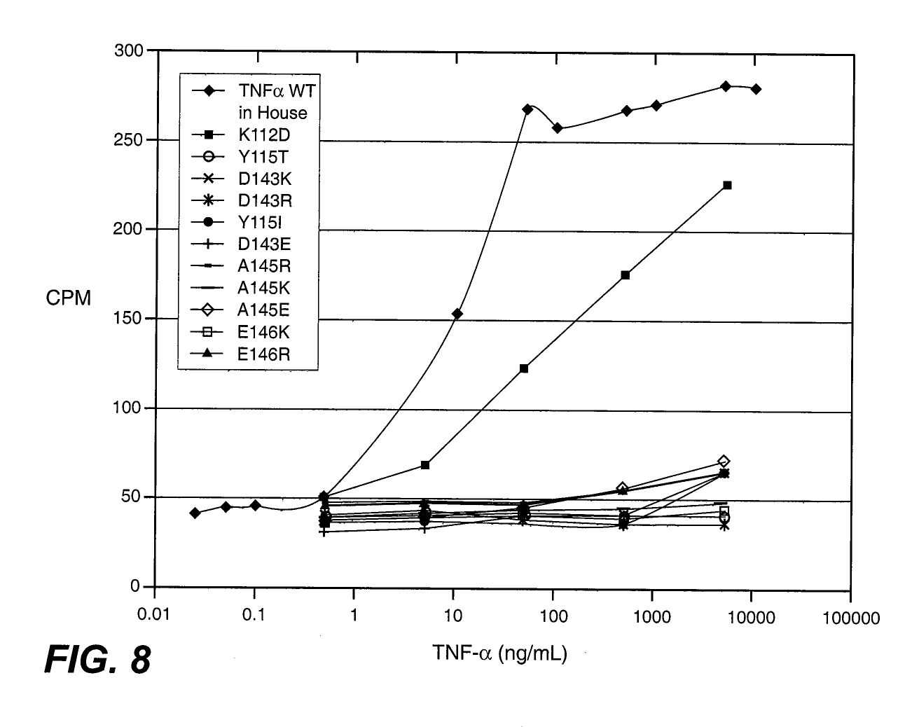

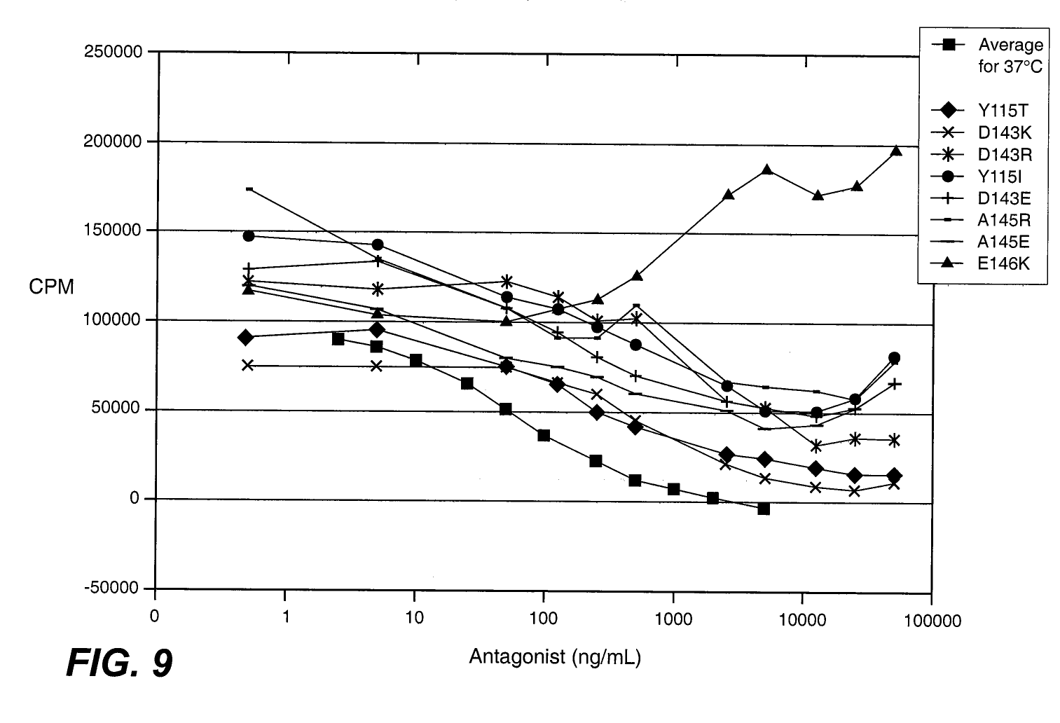

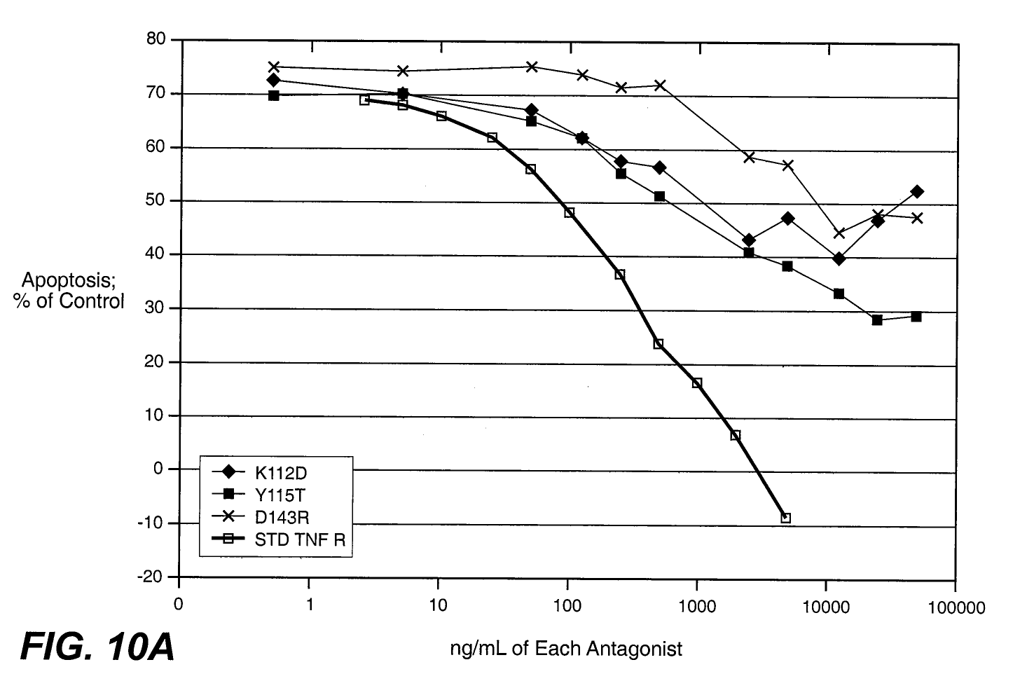

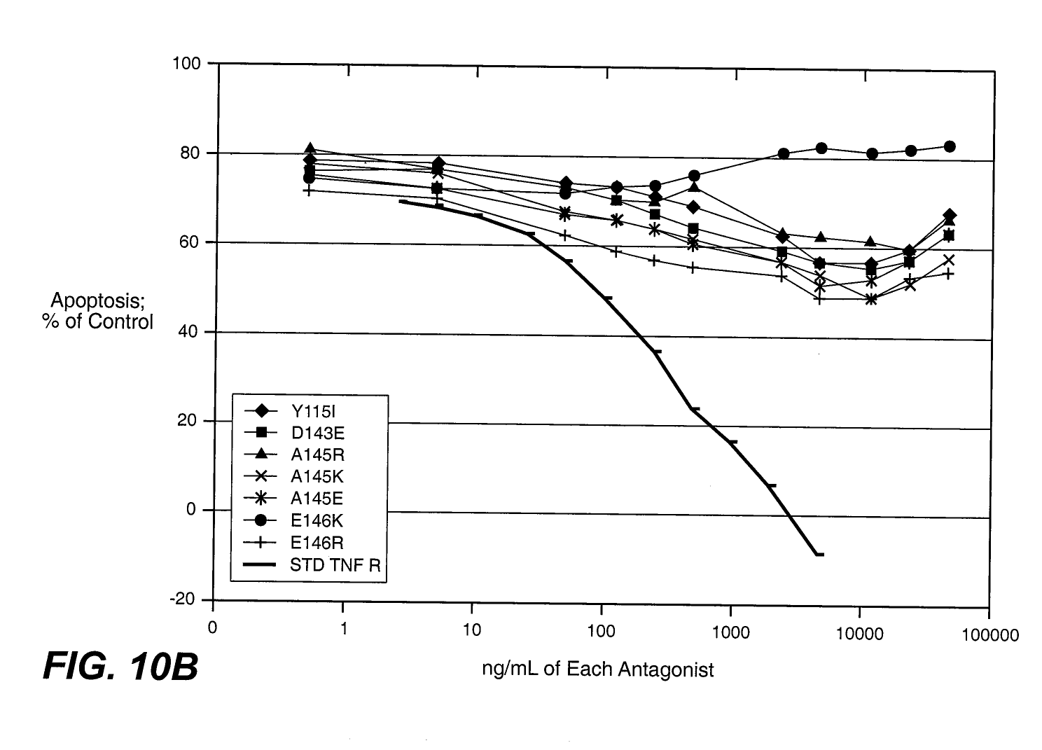

The TNF-α proteins may be from any number of organisms, with TNF-α proteins from mammals being particularly preferred. Suitable mammals include, but are not limited to, rodents (rats, mice, hamsters, guinea pigs, etc.), primates, farm animals (including sheep, goats, pigs, cows, horses, etc); and in the most preferred embodiment, from humans (the sequence of which is depicted in FIG. 6B). As will be appreciated by those in the art, TNF-α proteins based on TNF-α proteins from mammals other than humans may find use in animal models of human disease and treatment of domesticated animals.

The TNF proteins of the invention have modulated activity as compared to wild type proteins. In a preferred embodiment, variant TNF-α proteins exhibit decreased biological activity (e.g. antagonism) as compared to wild type TNF-α, including but not limited to, decreased binding to a receptor (p55, p75 or both), decreased activation and/or ultimately a loss of cytotoxic activity. By “cytotoxic activity” herein refers to the ability of a TNF-α variant to selectively kill or inhibit cells. Variant TNF-α proteins that exhibit less than 50% biological activity as compared to wild type are preferred. More preferred are variant TNF-α proteins that exhibit less than 25%, even more preferred are variant proteins that exhibit less than 15%, and most preferred are variant TNF-α proteins that exhibit less than 10% of a biological activity of wild-type TNF-α. Suitable assays include, but are not limited to, caspase assays, TNF-α cytotoxicity assays, DNA binding assays; transcription assays (using reporter constructs; see Stavridi, supra); size exclusion chromatography assays and radiolabeling/immuno-precipitation; see Corcoran et al., supra); and stability assays (including the use of circular dichroism (CD) assays and equilibrium studies; see Mateu, supra); all of which are incorporated by reference.

In one embodiment, at least one property critical for binding affinity of the variant TNF-α proteins is altered when compared to the same property of wild type TNF-α and in particular, variant TNF-α proteins with altered receptor affinity are preferred. Particularly preferred are variant TNF-α with altered affinity toward oligomerization to wild type TNF-α. Thus, the invention provides variant TNF-α proteins with altered binding affinities such that the variant TNF-α proteins will preferentially oligomerize with wild type TNF-α, but do not substantially interact with wild type TNF receptors, i.e., p55, p75. “Preferentially” in this case means that given equal amounts of variant TNF-α monomers and wild type TNF-α monomers, at least 25% of the resulting trimers are mixed trimers of variant and wild type TNF-α, with at least about 50% being preferred, and at least about 80-90% being particularly preferred. In other words, it is preferable that the variant TNF-α proteins of the invention have greater affinity for wild type TNF-α protein as compared to wild type TNF-α proteins. By “do not substantially interact with TNF receptors” is meant that the variant TNF-α proteins will not be able to associate with either the p55 or p75 receptors to significantly activate the receptor and initiate the TNF signaling pathway(s). In a preferred embodiment, at least a 50% decrease in receptor activation is seen, with greater than 50%, 76%, 80-90% being preferred.

Thus, the proteins of the invention are antagonists of wild type TNF-α. By “antagonists of wild type TNF-α” is meant that the variant TNF-α protein inhibits or significantly decreases at least one biological activity of wild-type TNF-α.

In some embodiments, the variants of the invention are antagonists of both soluble and transmembrane TNF-α. However, as described herein, some variant TNF-α proteins are antagonists of the activity of soluble TNF-α but do not substantially effect the activity of transmembrane TNF-α Thus, a reduction of activity of the heterotrimers for soluble TNF-α is as outlined above, with reductions in biological activity of at least 10%, 25, 50 75, 80, 90, 95, 99 or 100% all being preferred. However, some of the variants outlined herein comprise selective inhibition; that is, they inhibit soluble TNF-α activity but do not substantially inhibit transmembrane TNF-α. In these embodiments, it is preferred that at least 80%, 85, 90, 95, 98, 99 or 100% of the transmembrane TNF-α activity is maintained. This may also be expressed as a ratio; that is, selective inhibition can include a ratio of inhibition of soluble to transmembrane TNF-α. For example, variants that result in at least a 10:1 selective inhibition of soluble to transmembrane TNF-α activity are preferred, with 50:1, 100:1, 200:1, 500:1, 1000:1 or higher find particular use in the invention. Thus one embodiment utilizes variants, such as double mutants at positions 87/145 as outlined herein, that substantially inhibit or eliminate soluble TNF-α activity (for example by exchanging with homotrimeric wild-type to form heterotrimers that do not bind to TNF-α receptors or that bind but do not activate receptor signaling) but do not significantly effect (and preferably do not alter at all) transmembrane TNF-α activity. Without being bound by theory, the variants exhibiting such differential inhibition allow the descrease of inflammation without a corresponding loss in immune response.

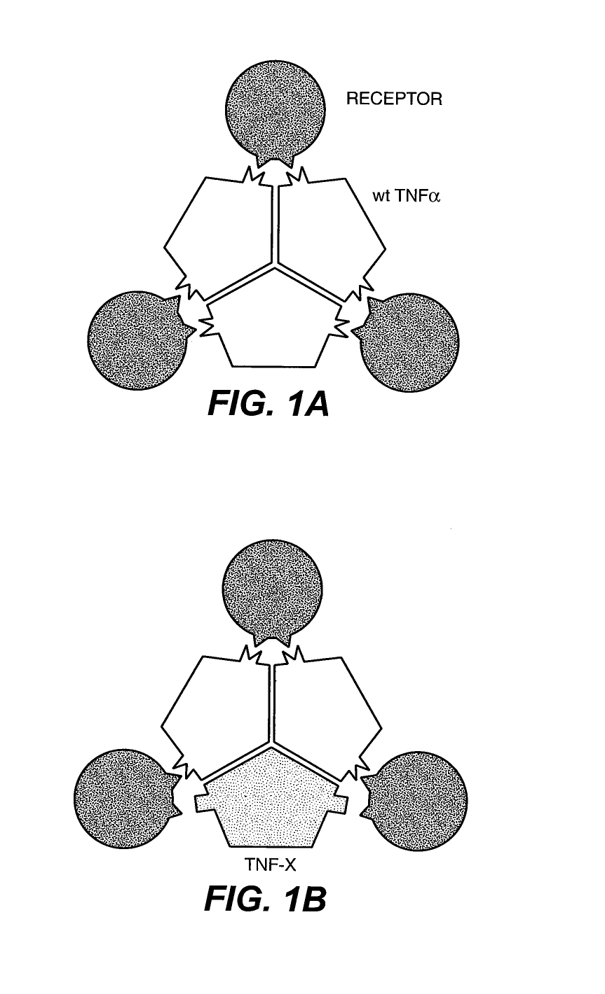

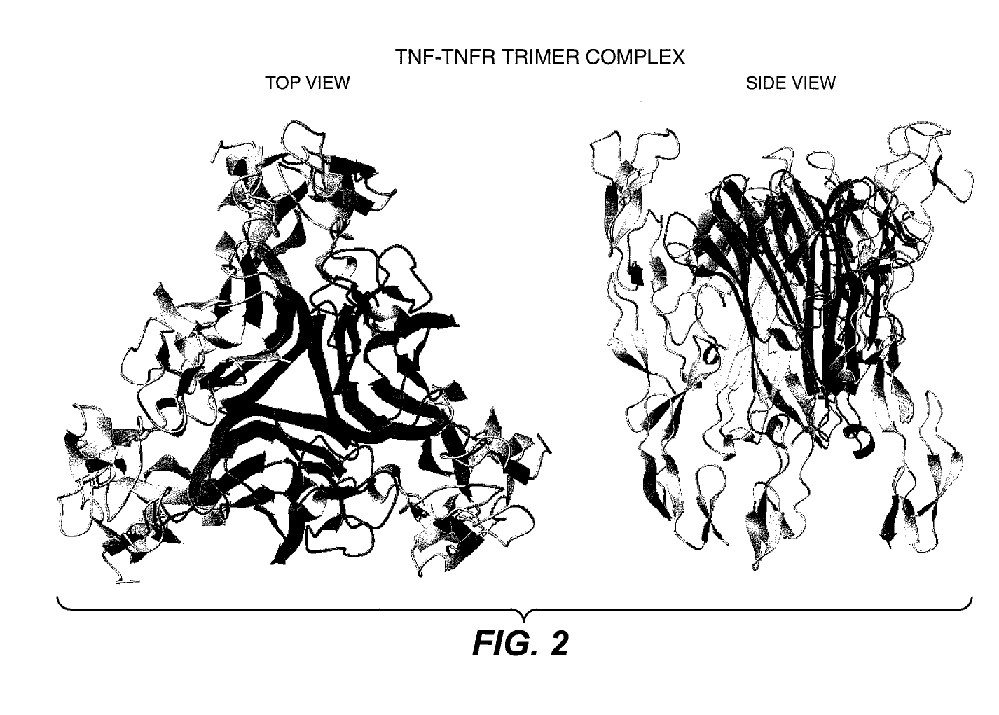

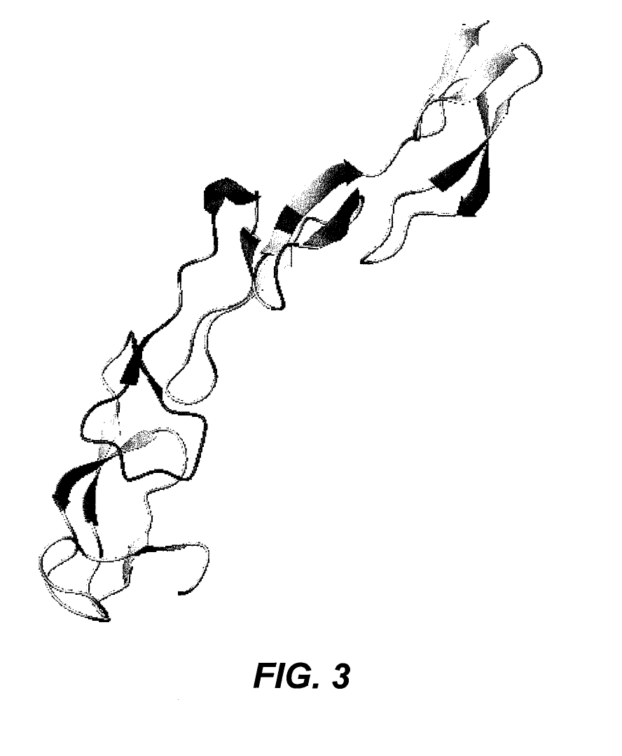

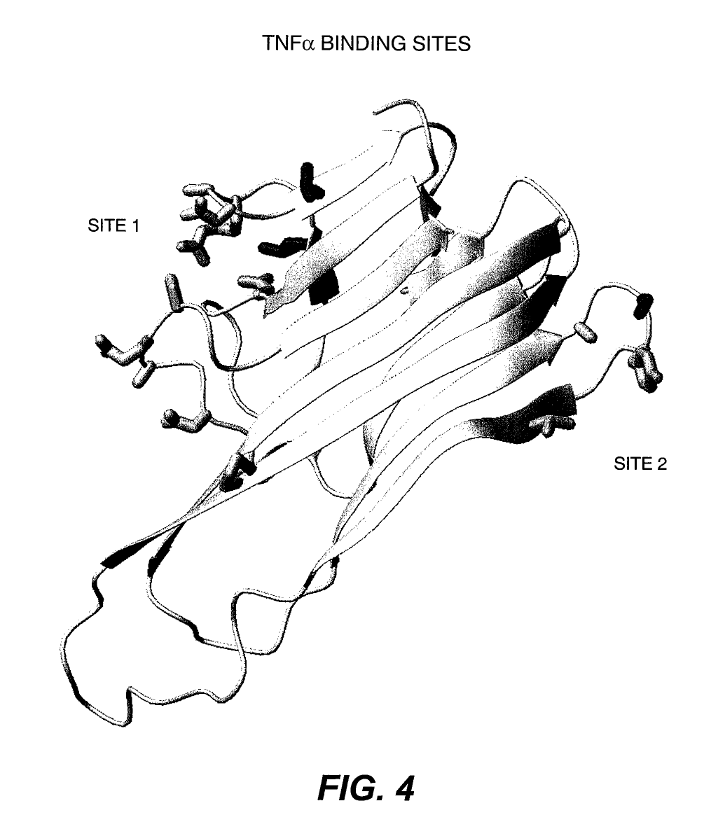

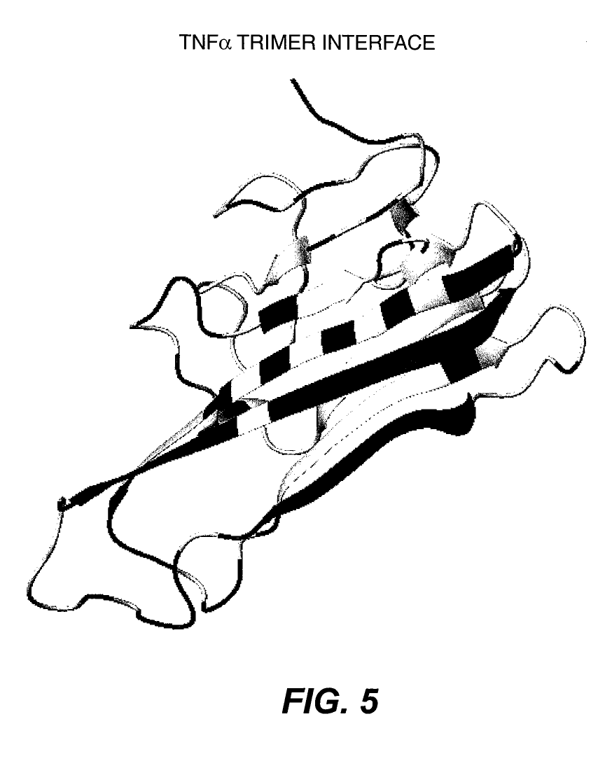

In one embodiment, the affected biological activity of the variants is the activation of receptor signaling by wild type TNF-α proteins. In a preferred embodiment, the variant TNF-α protein interacts with the wild type TNF-α protein such that the complex comprising the variant TNF-α and wild type TNF-α has reduced capacity to activiate (as outlined above for “substantial inhibition”), and in preferred embodiments is incapable of activating, one or both of the TNF receptors, i.e. p55 TNF-R or p75 TNF-R. In a preferred embodiment, the variant TNF-α protein is a variant TNF-α protein which functions as an antagonist of wild type TNF-α. Preferably, the variant TNF-α protein preferentially interacts with wild type TNF-α to form mixed trimers with the wild type protein such that receptor binding does not significantly occur and/or TNF-α signaling is not initiated (FIG. 1A). By mixed trimers is meant that monomers of wild type and variant TNF-α proteins interact to form heterotrimeric TNF-α (FIG. 5). Mixed trimers may comprise 1 variant TNF-α protein:2 wild type TNF-α proteins, 2 variant TNF-α proteins:1 wild type TNF-α protein. In some embodiments, trimers may be formed comprising only variant TNF-α proteins (FIG. 1B).

The variant TNF-α antagonist proteins of the invention are highly specific for TNF-α antagonism relative to TNF-beta antagonism. Additional characteristics include improved stability, pharmacokinetics, and high affinity for wild type TNF-α. Variants with higher affinity toward wild type TNF-α may be generated from variants exhibiting TNF-α antagonism as outlined above.

As outlined above, the invention provides variant TNF-α nucleic acids encoding variant TNF-α polypeptides. The variant TNF-α polypeptide preferably has at least one altered property as compared to the same property of the corresponding naturally occurring TNF polypeptide. The property of the variant TNF-α polypeptide is the result the PDA® analysis of the present invention. The term “altered property” or grammatical equivalents thereof in the context of a polypeptide, as used herein, further refers to any characteristic or attribute of a polypeptide that can be selected or detected and compared to the corresponding property of a naturally occurring protein. These properties include, but are not limited to cytotoxic activity; oxidative stability, substrate specificity, substrate binding or catalytic activity, thermal stability, alkaline stability, pH activity profile, resistance to proteolytic degradation, kinetic association (Kon) and dissociation (Koff) rate, protein folding, inducing an immune response, ability to bind to a ligand, ability to bind to a receptor, ability to be secreted, ability to be displayed on the surface of a cell, ability to oligomerize, ability to signal, ability to stimulate cell proliferation, ability to inhibit cell proliferation, ability to induce apoptosis, ability to be modified by phosphorylation or glycosylation, and the ability to treat disease.

Unless otherwise specified, a substantial change in any of the above-listed properties, when comparing the property of a variant TNF-α polypeptide to the property of a naturally occurring TNF protein is preferably at least a 20%, more preferably, 50%, more preferably at least a 2-fold increase or decrease. A change in cytotoxic activity is evidenced by at least a 75% or greater decrease in cell death initiated by a variant TNF-α protein as compared to wild type protein. A change in binding affinity is evidenced by at least a 5% or greater increase or decrease in binding affinity to wild type TNF receptor proteins or to wild type TNF-α.

A change in oxidative stability is evidenced by at least about 20%, more preferably at least 50% increase of activity of a variant TNF-α protein when exposed to various oxidizing conditions as compared to that of wild type TNF-α. Oxidative stability is measured by known procedures.

A change in alkaline stability is evidenced by at least about a 5% or greater increase or decrease (preferably increase) in the half-life of the activity of a variant TNF-α protein when exposed to increasing or decreasing pH conditions as compared to that of wild type TNF-α. Generally, alkaline stability is measured by known procedures.

A change in thermal stability is evidenced by at least about a 5% or greater increase or decrease (preferably increase) in the half-life of the activity of a variant TNF-α protein when exposed to a relatively high temperature and neutral pH as compared to that of wild type TNF-α. Generally, thermal stability is measured by known procedures.

Similarly, variant TNF-α proteins, for example are experimentally tested and validated in in vivo and in in vitro assays. Suitable assays include, but are not limited to, activity assays and binding assays. For example, TNF-α activity assays, such as detecting apoptosis via caspase activity can be used to screen for TNF-α variants that are antagonists of wild type TNF-α. Other assays include using the Sytox green nucleic acid stain to detect TNF-induced cell permeability in an Actinomycin-D sensitized cell line. As this stain is excluded from live cells, but penetrates dying cells, this assay also can be used to detect TNF-α variants that are agonists of wild-type TNF-α. By “agonists of “wild type TNF-α” is meant that the variant TNF-α protein enhances the activation of receptor signaling by wild type TNF-α proteins. Generally, variant TNF-α proteins that function as agonists of wild type TNF-α are not preferred. However, in some embodiments, variant TNF-α proteins that function as agonists of wild type TNF-α protein are preferred. An example of an NF kappaB assay is presented in Example 7.

In a preferred embodiment, binding affinities of variant TNF-α proteins as compared to wild type TNF-α proteins for naturally occurring TNF-α and TNF receptor proteins such as p55 and p75 are determined. Suitable assays include, but are not limited to, e.g., quantitative comparisons comparing kinetic and equilibrium binding constants. The kinetic association rate (Kon) and dissociation rate (Koff), and the equilibrium binding constants (Kd) may be determined using surface plasmon resonance on a BIAcore instrument following the standard procedure in the literature [Pearce et al., Biochemistry 38:81-89 (1999), incorporated by reference]. Examples of binding assays are described in Example 6.

In a preferred embodiment, the antigenic profile in the host animal of the variant TNF-α protein is similar, and preferably identical, to the antigenic profile of the host TNF-α; that is, the variant TNF-α protein does not significantly stimulate the host organism (e.g. the patient) to an immune response; that is, any immune response is not clinically relevant and there is no allergic response or neutralization of the protein by an antibody. That is, in a preferred embodiment, the variant TNF-α protein does not contain additional or different epitopes from the TNF-α. By “epitope” or “determinant” is meant a portion of a protein which will generate and/or bind an antibody. Thus, in most instances, no significant amounts of antibodies are generated to a variant TNF-α protein. In general, this is accomplished by not significantly altering surface residues, as outlined below nor by adding any amino acid residues on the surface which can become glycosylated, as novel glycosylation can result in an immune response.

The variant TNF-α proteins and nucleic acids of the invention are distinguishable from naturally occurring wild type TNF-α. By “naturally occurring” or “wild type” or grammatical equivalents, is meant an amino acid sequence or a nucleotide sequence that is found in nature and includes allelic variations; that is, an amino acid sequence or a nucleotide sequence that usually has not been intentionally modified. Accordingly, by “non-naturally occurring” or “synthetic” or “recombinant” or grammatical equivalents thereof, is meant an amino acid sequence or a nucleotide sequence that is not found in nature; that is, an amino acid sequence or a nucleotide sequence that usually has been intentionally modified. It is understood that once a recombinant nucleic acid is made and reintroduced into a host cell or organism, it will replicate non-recombinantly, i.e., using the in vivo cellular machinery of the host cell rather than in vitro manipulations, however, such nucleic acids, once produced recombinantly, although subsequently replicated non-recombinantly, are still considered recombinant for the purpose of the invention. Representative amino acid and nucleotide sequences of a naturally occurring human TNF-α are shown in FIGS. 6A and 6B. It should be noted, that unless otherwise stated, all positional numbering of variant TNF-α proteins and variant TNF-α nucleic acids is based on these sequences. That is, as will be appreciated by those in the art, an alignment of TNF-α proteins and variant TNF-α proteins may be done using standard programs, as is outlined below, with the identification of “equivalent” positions between the two proteins. Thus, the variant TNF-α proteins and nucleic acids of the invention are non-naturally occurring; that is, they do not exist in nature.

Thus, in a preferred embodiment, the variant TNF-α protein has an amino acid sequence that differs from a wild type TNF-α sequence by at least 1 amino acid, with from 1, 2, 3, 4, 5, 6, 7, 8, 9 and 10 amino acids all contemplated, or higher. Expressed as a percentage, the variant TNF-α proteins of the invention preferably are greater than 90% identical to wild-type, with greater than 95, 97, 98 and 99% all being contemplated. Stated differently, based on the human TNF sequence of FIG. 6B, variant TNF-α proteins have at least about 1 residue that differs from the human TNF-α sequence, with at least about 2, 3, 4, or 5 different residues. Preferred variant TNF-α proteins have 3 to 5 different residues.

Homology in this context means sequence similarity or identity, with identity being preferred. As is known in the art, a number of different programs may be used to identify whether a protein (or nucleic acid as discussed below) has sequence identity or similarity to a known sequence. Sequence identity and/or similarity is determined using standard techniques known in the art, including, but not limited to, the local sequence identity algorithm of Smith & Waterman, Adv. Appl. Math., 2:482 (1981), by the sequence identity alignment algorithm of Needleman & Wunsch, J. Mol. Biol., 48:443 (1970), by the search for similarity method of Pearson & Lipman, Proc. Natl. Acad. Sci. U.S.A., 85:2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res., 12:387-395 (1984), preferably using the default settings, or by inspection. Preferably, percent identity is calculated by FastDB based upon the following parameters: mismatch penalty of 1; gap penalty of 1; gap size penalty of 0.33; and joining penalty of 30, “Current Methods in Sequence Comparison and Analysis,” Macromolecule Sequencing and Synthesis, Selected Methods and Applications, pp 127-149 (1988), Alan R. Liss, Inc, all of which are incorporated by reference.

An example of a useful algorithm is PILEUP. PILEUP creates a multiple sequence alignment from a group of related sequences using progressive, pair wise alignments. It may also plot a tree showing the clustering relationships used to create the alignment. PILEUP uses a simplification of the progressive alignment method of Feng & Doolittle, J. Mol. Evol. 35:351-360 (1987); the method is similar to that described by Higgins & Sharp CABIOS 5:151-153 (1989), both incorporated by reference. Useful PILEUP parameters including a default gap weight of 3.00, a default gap length weight of 0.10, and weighted end gaps.

Another example of a useful algorithm is the BLAST algorithm, described in: Altschul et al., J. Mol. Biol. 215, 403-410, (1990); Altschul et al., Nucleic Acids Res. 25:3389-3402 (1997); and Karlin et al., Proc. Natl. Acad. Sci. U.S.A. 90:5873-5787 (1993), both incorporated by reference. A particularly useful BLAST program is the WU-BLAST-2 program which was obtained from Altschul et al., Methods in Enzymology, 266:460-480 (1996); http://blast.wustliedu/blast/README.html]. WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST, as reported by Altschul et al., Nucl. Acids Res., 25:3389-3402, incorporated by reference. Gapped BLAST uses BLOSUM-62 substitution scores; threshold T parameter set to 9; the two-hit method to trigger ungapped extensions; charges gap lengths of k a cost of 10+k; Xu set to 16, and Xg set to 40 for database search stage and to 67 for the output stage of the algorithms. Gapped alignments are triggered by a score corresponding to −22 bits.

A % amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored). In a similar manner, “percent (%) nucleic acid sequence identity” with respect to the coding sequence of the polypeptides identified is defined as the percentage of nucleotide residues in a candidate sequence that are identical with the nucleotide residues in the coding sequence of the cell cycle protein. A preferred method utilizes the BLASTN module of WU-BLAST-2 set to the default parameters, with overlap span and overlap fraction set to 1 and 0.125, respectively.

The alignment may include the introduction of gaps in the sequences to be aligned. In addition, for sequences which contain either more or fewer amino acids than the protein encoded by the sequence of FIG. 6B, it is understood that in one embodiment, the percentage of sequence identity will be determined based on the number of identical amino acids in relation to the total number of amino acids. Thus, for example, sequence identity of sequences shorter than that shown in FIG. 6, as discussed below, will be determined using the number of amino acids in the shorter sequence, in one embodiment. In percent identity calculations relative weight is not assigned to various manifestations of sequence variation, such as, insertions, deletions, substitutions, etc.

In one embodiment, only identities are scored positively (+1) and all forms of sequence variation including gaps are assigned a value of “0”, which obviates the need for a weighted scale or parameters as described below for sequence similarity calculations. Percent sequence identity may be calculated, for example, by dividing the number of matching identical residues by the total number of residues of the “shorter” sequence in the aligned region and multiplying by 100. The “longer” sequence is the one having the most actual residues in the aligned region.

Thus, the variant TNF-α proteins of the present invention may be shorter or longer than the amino acid sequence shown in FIG. 6B. As used in this invention, “wild type TNF-α” is a native mammalian protein (preferably human). TNF-α is polymorphic. An example of the amino acid sequences shown in FIG. 6B. Thus, in a preferred embodiment, included within the definition of variant TNF proteins are portions or fragments of the sequences depicted herein. Fragments of variant TNF-α proteins are considered variant TNF-α proteins if a) they share at least one antigenic epitope; b) have at least the indicated homology; c) and preferably have variant TNF-α biological activity as defined herein.

In a preferred embodiment, as is more fully outlined below, the variant TNF-α proteins include further amino acid variations, as compared to a wild type TNF-α, than those outlined herein. In addition, any of the variations depicted herein may be combined in any way to form additional novel variant TNF-α proteins. In addition, variant TNF-α proteins may be made that are longer than those depicted in the figures, for example, by the addition of epitope or purification tags, as outlined herein, the addition of other fusion sequences, etc.

TNF-α proteins may be fused to, for example, to other therapeutic proteins or to other proteins such as Fc or serum albumin for therapeutic or pharmacokinetic purposes. In this embodiment, a TNF-α protein of the present invention is is operably linked to a fusion partner. The fusion partner may be any moiety that provides an intended therapeutic or pharmacokinetic effect. Examples of fusion partners include but are not limited to Human Serum Albumin, a therapeutic agent, a cytotoxic or cytotoxic molecule, radionucleotide, and an Fc, etc. As used herein, an Fc fusion is synonymous with the terms “immunoadhesin”, “lg fusion”, “lg chimera”, and “receptor globulin” as used in the prior art (Chamow et al., 1996, Trends Biotechnol 14:52-60; Ashkenazi et al., 1997, Curr Opin Immunol 9:195200, both incorporated by reference). An Fc fusion combines the Fc region of an immunoglobulin with the target-binding region of a TNF-α protein, for example. See for example U.S. Pat. Nos. 5,766,883 and 5,876,969, both of which are incorporated by reference.

the present invention provides compositions comprising a variant human TNF-α monomer comprising the formula (Vb stands for “variable” and Fx stands for “fixed”):
Vb(1)-Fx(2-9)-Vb(10)-Fx(11-20)-Vb(21)-Fx(22)-Vb(23)-Vb(24)-Vb(25)-Fx(26)-Vb(27)-Fx(28-29)-Vb(30)-Vb(31)-Vb(32)-Vb(33)-Vb(34)-Vb(35)-Fx(36-41)-Vb(42)-Fx(43)-Vb(44)-Vb(45)-Fx(46-56)-Vb(57)-Fx(58-64)-Vb(65)-Vb(66)-Vb(67)-Fx(68-75)-Vb(75)-Fx(76-83)-Vb(84)-Fx(85)-Vb(86)-Vb(87)-Vb(88)-Fx(89)-Vb(90)-Vb(91)-Fx(92-96)-Vb(97)-Fx(98-100)-Vb(101)-Fx(102-106)-Vb(107)-Vb(108)-Fx(109)-Vb(110)-Vb(111)-Vb(112)-Fx(113-114)-Vb(115)-Fx(116-127)-Vb(128)-Fx(129-139)-Vb(140)-Fx(141-142)-Vb(143)-Vb(144)-Vb(145)-Vb(146)-Vb(147),
   wherein:
   Vb(1) is selected from the group consisting of V, L and M; Fx(2-9) comprises the human amino acid sequence of TNF-α at positions 2-9_(SEQ ID NO.9); Vb(10) is selected from the group consisting of D and C; Fx(11-20) comprises the human amino acid sequence of TNF-α at positions 11-20 (SEQ ID NO.10); Vb(21) is selected from the group consisting of Q, C and R; Fx(22) is the amino acid at position 22 of human TNF-α; Vb(23) is selected from the group consisting of E and C; Vb(24) is selected from the group consisting of G and C; Vb(25) is selected from the group consisting of Q and C; Fx(26) comprises the human amino acid sequence of TNF-α at position 26; Vb(27) is selected from the group consisting of Q and C; Fx(28-29) comprises the human amino acid sequence of TNF-α at positions 28-29; Vb(30) is selected from the group consisting of N and D; Vb(31) is selected from the group consisting of R, C, I, D and E; Vb(32) is selected from the group consisting of R, D, E and S; Vb(33) is selected from the group consisting of A and E; Vb(34) is selected from the group consisting of N, E and V; Vb(35) is selected from the group consisting of A and S; Fx(36-41) comprises the human amino acid sequence of TNF-α at positions 36-41 (SEQ ID NO.11); Vb(42) is selected from the group consisting of E and C; Fx(43) comprises the human amino acid sequence of TNF-α at position 43; Vb(44) is selected from the group consisting of R and C; Vb(45) is selected from the group consisting of D and C; Fx(46-56) comprises the human amino acid sequence of TNF-α at positions 46-56 (SEQ ID NO.12); Vb(57) is selected from the group consisting of L, F, W and Y; Fx(58-64) comprises the human amino acid sequence of TNF-α at positions 58-64 (SEQ ID NO.13); Vb(65) is selected from the group consisting of K, D, E, I, M, N, Q, T, S V and W; Vb(66) is selected from the group consisting of G, K and Q; Vb(67) is selected from the group consisting of Q, D, K, R, S, W and Y; Fx(68) comprises the human amino acid sequence of TNF-α at positions 68; Vb(69) is selected from the group consisting of C and V; Fx(70-74) comprises the human amino acid sequence of TNF-α at positions 70-74 (SEQ ID NO. 14); Vb(75) is selected from the group consisting of L, E, K and Q; Fx(76-83) comprises the human amino acid sequence of TNF-α at positions 76-83 (SEQ ID NO.15); Vb(84) is selected from the group consisting of A and V; Fx(85) is the amino acid at position 85 of human TNF-α; Vb(86) is selected from the group consisting of S, Q and R; Vb(87) is selected from the group consisting of Y, H and R; Vb(88) is selected from the group consisting of Q and C; Fx(89) comprises the human amino acid sequence of TNF-α at position 89; Vb(90) is selected from the group consisting of K and C; Vb(91) is selected from the group consisting of V and E; Fx(92-96) comprises the human amino acid sequence of TNF-α at positions 92-96 (SEQ ID NO.16); Vb(97) is selected from the group consisting of 1, R and T; Fx(98-100) comprises the human amino acid sequence of TNF-α at positions 98-100; Vb(101) is selected from the group consisting of C and A; Fx(102-106) comprises the human amino acid sequence of TNF-α at positions 102-106 (SEQ ID NO. 17); Vb(107) is selected from the group consisting of I and C; Vb(108) is selected from the group consisting of G and C; Fx(109) comprises the human amino acid sequence of TNF-α at position 109; Vb(110) is selected from the group consisting of E and C; Vb(111) is selected from the group consisting of A, R and E; Vb(112) is selected from the group consisting of K, D and E; Fx(113-114) comprises the human amino acid sequence of TNF-α at positions 113-114; Vb(115) is selected from the group consisting of Y, D, E, F, H, I, K, L, M, N, Q, R, S, T and W; Fx(116-127) comprises the human amino acid sequence of TNF-α at positions 116-127 (SEQ ID NO.18); Vb(128) is selected from the group consisting of K and C; Fx(129-139) comprises the human amino acid sequence of TNF-α at positions 129-139 (SEQ ID NO.19); Vb(140) is selected from the group consisting of D, K and R; Fx(141-142) comprises the human amino acid sequence of TNF-α at positions 141-142; Vb(143) is selected from the group consisting of D, E, K, L, R, N, Q and S; Vb(144) is selected from the group consisting of F and N; Vb(145) is selected from the group consisting of A, D, E, F, H, K, M, N, Q, R, S, T and Y; Vb(146) is selected from the group consisting of E, K, L, M, N, R and S; and Vb(147) is selected from the group consisting of S and R.

In a preferred embodiment, the variant TNF-α proteins comprise residues selected from the following positions 21, 23, 30, 31, 32, 33, 34, 35, 57, 65, 66, 67, 69, 75, 84, 86, 87, 91, 97, 101, 111, 112, 115, 140, 143, 144, 145, 146, and 147. Preferred amino acids for each position, including the human TNF-α residues, are shown in FIG. 7. Thus, for example, at position 143, preferred amino acids are Glu, Asn, Gin, Ser, Arg, and Lys; etc. Preferred changes include: Q21C, Q21R, E23C, N34E, V91E, Q21R, N30D, R31C, R31I, R31D, R31E, R32D, R32E, R32S, A33E, N34E, N34V, A35S, D45C, L57F, L57W, L57Y, K65D, K65E, K651, K65M, K65N, K65Q, K65T, K65S, K65V, K65W, G66K, G66Q, Q67D, Q67K, Q67R, Q67S, Q67W, Q67Y, C69V, L75E, L75K, L75Q, A84V, S86Q, S86R, Y87H, Y87R, V91E, 197R, 197T, C101A, A111R, A111E, K112D, K112E, Y115D, Y115E, Y115F, Y115H, Y1151, Y115K, Y115L, Y115M, Y115N, Y115Q, Y115R, Y115S, Y115T, Y115W, D140K, D140R, D143E, D143K, D143L, D143R, D143N, D143Q, D143R, D143S, F144N, A145D, A145E, A145F, A145H, A145K, A145M, A145N, A145Q, A145R, A145S, A145T, A145Y, E146K, E146L, E146M, E146N, E146R, E146S and S147R. These may be done either individually or in combination, with any combination being possible. However, as outlined herein, preferred embodiments utilize at least 1 to 5, and preferably more, positions in each variant TNF-α protein.

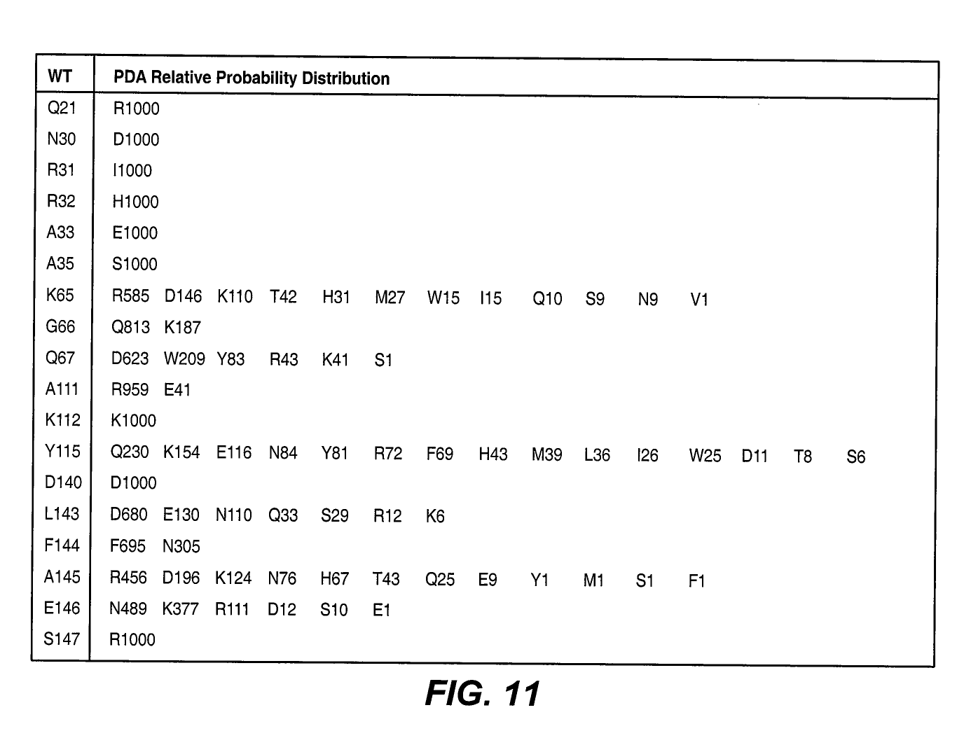

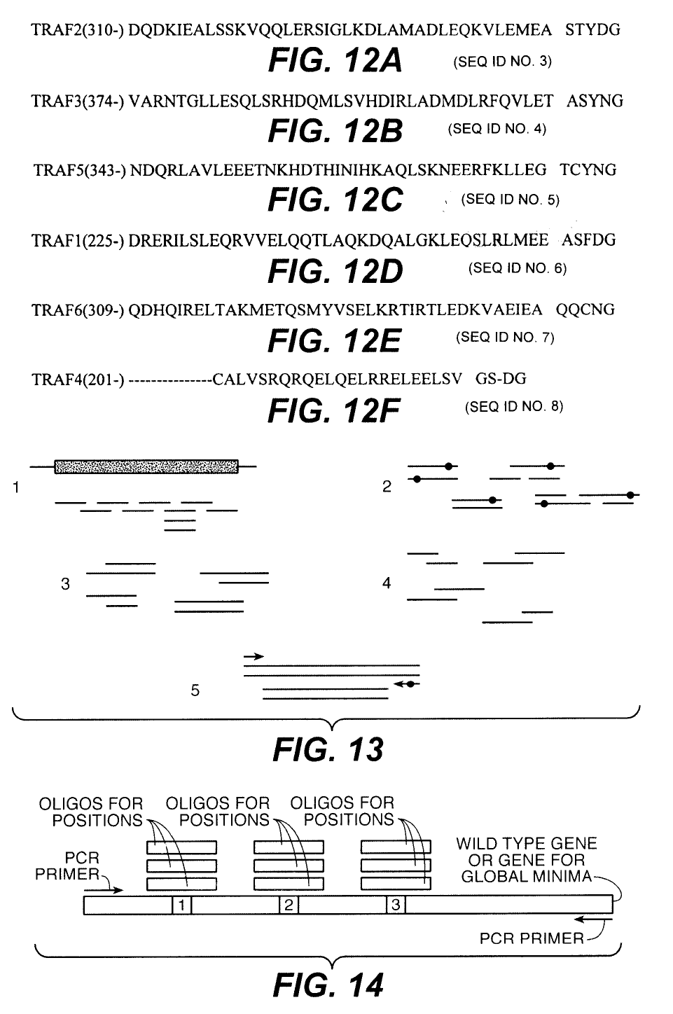

For purposes of the present invention, the areas of the wild type or naturally occurring TNF-α molecule to be modified are selected from the group consisting of the Large Domain (also known as II), Small Domain (also known as 1), the DE loop, and the trimer interface. The Large Domain, the Small Domain and the DE loop are the receptor interaction domains. The modifications may be made solely in one of these areas or in any combination of these areas. The Large Domain preferred positions to be varied include: 21, 30, 31, 32, 33, 35, 65, 66, 67, 111, 112, 115, 140, 143, 144, 145, 146 and/or 147 (FIG. 11). For the Small Domain, the preferred positions to be modified are 75 and/or 97. For the DE Loop, the preferred position modifications are 84, 86, 87 and/or 91. The Trimer Interface has preferred double variants including positions 34 and 91 as well as at position 57. In a preferred embodiment, substitutions at multiple receptor interaction and/or trimerization domains may be combined. Examples include, but are not limited to, simultaneous substitution of amino acids at the large and small domains (e.g. A145R and 197T), large domain and DE loop (A145R and Y87H), and large domain and trimerization domain (A145R and L57F). Additional examples include any and all combinations, e.g., 197T and Y87H (small domain and DE loop). More specifically, theses variants may be in the form of single point variants, for example K112D, Y115K, Y1151, Y115T, A145E or A145R. These single point variants may be combined, for example, Y1151 and A145E, or Y115I and A145R, or Y115T and A145R or Y1151 and A145E; or any other combination.

Preferred double point variant positions include 57, 75, 86, 87, 97, 115, 143,145, and 146; in any combination. In addition, double point variants may be generated including L57F and one of Y1151, Y115Q, Y115T, D143K, D143R, D143E, A145E, A145R, E146K or E146R. Other preferred double variants are Y115Q and at least one of D143N, D143Q, A145K, A145R, or E146K; Y115M and at least one of D143N, D143Q, A145K, A145R or E146K; and L57F and at least one of A145E or 146R; K65D and either D143K or D143R, K65E and either D143K or D143R, Y115Q and any of L75Q, L57W, L57Y, L57F, 197R, 197T, S86Q, D143N, E146K, A145R and 197T, A145R and either Y87R or Y87H; N34E and V91E; L75E and Y115Q; L75Q and Y115Q; L75E and A145R; and L75Q and A145R.

Further, triple point variants may be generated. Preferred positions include 34, 75, 87, 91, 115, 143, 145 and 146. Examples of triple point variants include V91 E, N34E and one of Y115I, Y115T, D143K, D143R, A145R, A145E E146K, and E146R. Other triple point variants include L75E and Y87H and at least one of Y115Q, A145R, Also, L75K, Y87H and Y115Q. More preferred are the triple point variants V91 E, N34E and either A145R or A145E.

In a preferred embodiment, the variant TNF-α proteins of the invention are human TNF-α conformers. By “conformer” is meant a protein that has a protein backbone 3-D structure that is virtually the same but has significant differences in the amino acid side chains. That is, the variant TNF-α proteins of the invention define a conformer set, wherein all of the proteins of the set share a backbone structure and yet have sequences that differ by at least 1-3-5%. The three dimensional backbone structure of a variant INF-α protein thus substantially corresponds to the three-dimensional backbone structure of human TNF-α. “Backbone” in this context means the non-side chain atoms: the nitrogen, carbonyl carbon and oxygen, and the a-carbon, and the hydrogens attached to the nitrogen and a-carbon. To be considered a conformer, a protein must have backbone atoms that are no more than 2 Angstroms RMSD from the human TNF-α structure, with no more than 1.5 Angstroms RMSD being preferred, and no more than 1 Angstrom RMSD being particularly preferred. In general, these distances may be determined in two ways. In one embodiment, each potential conformer is crystallized and its three-dimensional structure determined. Alternatively, as the former is quite tedious, the sequence of each potential conformer is run in the PDA™ technology program to determine whether it is a conformer.

Variant TNF-α proteins may also be identified as being encoded by variant TNF-α nucleic acids. In the case of the nucleic acid, the overall homology of the nucleic acid sequence is commensurate with amino acid homology but takes into account the degeneracy in the genetic code and codon bias of different organisms. Accordingly, the nucleic acid sequence homology may be either lower or higher than that of the protein sequence, with lower homology being preferred. In a preferred embodiment, a variant TNF-α nucleic acid encodes a variant TNF-α protein. As will be appreciated by those in the art, due to the degeneracy of the genetic code, an extremely large number of nucleic acids may be made, all of which encode the variant TNF-α proteins of the present invention. Thus, having identified a particular amino acid sequence, those skilled in the art could make any number of different nucleic acids, by simply modifying the sequence of one or more codons in a way which does not change the amino acid sequence of the variant TNF-α.

In one embodiment, the nucleic acid homology is determined through hybridization studies. Thus, for example, nucleic acids which hybridize under high stringency to the nucleic acid sequence shown in FIG. 6A or its complement and encode a variant TNF-α protein is considered a variant TNF-α gene. High stringency conditions are known in the art; see for example Maniatis et al., Molecular Cloning: A Laboratory Manual, 2d Edition, 1989, and Short Protocols in Molecular Biology, ed. Ausubel, et al., both of which are hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993), incorporated by reference. Generally, stringent conditions are selected to be about 5-10 degrees C. lower than the thermal melting point (Tm) for the specific sequence at a defined ionic strength and pH. The TM is the temperature (under defined ionic strength, pH and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30 degrees C. for short probes (e.g. 10 to 50 nucleotides) and at least about 60 degrees C. for long probes (e.g. greater than 50 nucleotides). Stringent conditions may also be achieved with the addition of destabilizing agents such as formamide. In another embodiment, less stringent hybridization conditions are used; for example, moderate or low stringency conditions may be used, as are known in the art; see Maniatis and Ausubel, supra, and Tijssen, supra.

The variant TNF-α proteins and nucleic acids of the present invention are recombinant. As used herein, “nucleic acid” may refer to either DNA or RNA, or molecules which contain both deoxy- and ribonucleotides. The nucleic acids include genomic DNA, cDNA and oligonucleotides including sense and anti-sense nucleic acids. Such nucleic acids may also contain modifications in the ribose-phosphate backbone to increase stability and half-life of such molecules in physiological environments. The nucleic acid may be double stranded, single stranded, or contain portions of both double stranded or single stranded sequence. As will be appreciated by those in the art, the depiction of a single strand (“Watson”) also defines the sequence of the other strand (“Crick”); thus the sequence depicted in FIG. 6 also includes the complement of the sequence. By the term “recombinant nucleic acid” is meant nucleic acid, originally formed in vitro, in general, by the manipulation of nucleic acid by endonucleases, in a form not normally found in nature. Thus an isolated variant TNF-α nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e. using the in vivo cellular machinery of the host cell rather than in vitro manipulations; however, such nucleic acids, once produced recombinantly, although subsequently replicated non-recombinantly, are still considered recombinant for the purposes of the invention.

Similarly, a “recombinant protein” is a protein made using recombinant techniques, i.e. through the expression of a recombinant nucleic acid as depicted above. A recombinant protein is distinguished from naturally occurring protein by at least one or more characteristics. For example, the protein may be isolated or purified away from some or all of the proteins and compounds with which it is normally associated in its wild-type host, and thus may be substantially pure. For example, an isolated protein is unaccompanied by at least some of the material with which it is normally associated in its natural state, preferably constituting at least about 0.5%, more preferably at least about 5% by weight of the total protein in a given sample. A substantially pure protein comprises at least about 75% by weight of the total protein, with at least about 80% being preferred, and at least about 90% being particularly preferred. The definition includes the production of a variant TNF-α protein from one organism in a different organism or host cell. Alternatively, the protein may be made at a significantly higher concentration than is normally seen, through the use of a inducible promoter or high expression promoter, such that the protein is made at increased concentration levels. Furthermore, all of the variant TNF-α proteins outlined herein are in a form not normally found in nature, as they contain amino acid substitutions, insertions and deletions, with substitutions being preferred, as discussed below.

Also included within the definition of variant TNF-α proteins of the present invention are amino acid sequence variants of the variant TNF-α sequences outlined herein and shown in the Figures. That is, the variant TNF-α proteins may contain additional variable positions as compared to human TNF-α. These variants fall into one or more of three classes: substitutional, insertional or deletional variants. These variants ordinarily are prepared by site-specific mutagenesis of nucleotides in the DNA encoding a variant TNF-α protein, using cassette or PCR mutagenesis or other techniques well known in the art, to produce DNA encoding the variant, and thereafter expressing the DNA in recombinant cell culture as outlined above. However, variant TNF-α protein fragments having up to about 100-150 residues may be prepared by in vitro synthesis using established techniques. Amino acid sequence variants are characterized by the predetermined nature of the variation, a feature that sets them apart from naturally occurring allelic or interspecies variation of the variant TNF-α protein amino acid sequence. The variants typically exhibit the same qualitative biological activity as the naturally occurring analogue; although variants can also be selected which have modified characteristics as will be more fully outlined below.

While the site or region for introducing an amino acid sequence variation is predetermined, the mutation per se need not be predetermined. For example, in order to optimize the performance of a mutation at a given site, random mutagenesis may be conducted at the target codon or region and the expressed variant TNF-α proteins screened for the optimal combination of desired activity. Techniques for making substitution mutations at predetermined sites in DNA having a known sequence are well known, for example, M13 primer mutagenesis and PCR mutagenesis. Screening of the mutants is done using assays of variant TNF-α protein activities.

Amino acid substitutions are typically of single residues; insertions usually will be on the order of from about 1 to 20 amino acids, although considerably larger insertions may be tolerated. Deletions range from about 1 to about 20 residues, although in some cases deletions may be much larger.

Substitutions, deletions, insertions or any combination thereof may be used to arrive at a final derivative. Generally these changes are done on a few amino acids to minimize the alteration of the molecule. However, larger changes may be tolerated in certain circumstances. When small alterations in the characteristics of the variant TNF-α protein are desired, substitutions are often made in accordance with the following: Ala to Ser; Arg to Lys; Asn to Gln, His; Asp to Glu; Cys to Ser, Ala; Gln to Asn; Glu to Asp; Gly to Pro; His to Asn, Gin; Ile to Leu, Val; Leu to lie, Val; Lys to Arg, Gln, Glu; Met to Leu, lie; Phe to Met, Leu, Tyr; Ser to Thr; Thr to Ser; Trp to Tyr; Tyr to Trp, Phe; Val to lie, Leu.

Substantial changes in function or immunological identity are made by selecting substitutions that are less conservative than those shown above. For example, substitutions may be made which more significantly affect: the structure of the polypeptide backbone in the area of the alteration, for example the alpha-helical or beta-sheet structure; the charge or hydrophobicity of the molecule at the target site; or the bulk of the side chain. The substitutions which in general are expected to produce the greatest changes in the polypeptide's properties are those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g. lysyl, arginyl, or histidyl, is substituted for (or by) an electronegative residue, e.g. glutamyl or aspartyl; or (d) a residue having a bulky side chain, e.g. phenylalanine, is substituted for (or by) one not having a side chain, e.g. glycine.

The variants typically exhibit the same qualitative biological activity and will elicit the same immune response as the original variant TNF-α protein, although variants also are selected to modify the characteristics of the variant TNF-α proteins as needed. Alternatively, the variant may be designed such that the biological activity of the variant TNF-α protein is altered. For example, glycosylation and/or pegylation sites may be altered or removed. Similarly, the biological function may be altered; for example, in some instances it may be desirable to have more or less potent TNF-α activity.

The variant TNF-α proteins and nucleic acids of the invention can be made in a number of ways. Individual nucleic acids and proteins can be made as known in the art and outlined below. Alternatively, libraries of variant TNF-α proteins can be made for testing. In a preferred embodiment, sets or libraries of variant TNF-α proteins are generated from a probability distribution table. As outlined herein, there are a variety of methods of generating a probability distribution table, including using PDA® technology calculations, sequence alignments, forcefield calculations such as SCMF calculations, etc. In addition, the probability distribution can be used to generate information entropy scores for each position, as a measure of the mutational frequency observed in the library. In this embodiment, the frequency of each amino acid residue at each variable position in the list is identified. Frequencies may be thresholded, wherein any variant frequency lower than a cutoff is set to zero. This cutoff is preferably 1%, 2%, 5%, 10% or 20%, with 10% being particularly preferred. These frequencies are then built into the variant TNF-α library. That is, as above, these variable positions are collected and all possible combinations are generated, but the amino acid residues that “fill” the library are utilized on a frequency basis. Thus, in a non-frequency based library, a variable position that has 5 possible residues will have 20% of the proteins comprising that variable position with the first possible residue, 20% with the second, etc. However, in a frequency based library, a variable position that has 5 possible residues with frequencies of 10%, 15%, 25%, 30% and 20%, respectively, will have 10% of the proteins comprising that variable position with the first possible residue, 15% of the proteins with the second residue, 25% with the third, etc. As will be appreciated by those in the art, the actual frequency may depend on the method used to actually generate the proteins; for example, exact frequencies may be possible when the proteins are synthesized. However, when the frequency-based primer system outlined below is used, the actual frequencies at each position will vary, as outlined below.

In another embodiment, the novel trimeric complexes that are formed will act as competitive inhibitors of normal receptor signaling without the signaling produced by divalent binders. The heterotrimer complex of the present invention has a single, monovalent receptor binding site.

The receptor binding interface of trimeric TNF ligands has two sides, each contributed by a different monomer subunit. One side consists of the “Large Domain” while the other is made up of the “Small Domain” and the “DE Loop”. Disruption of receptor binding and consequent agonist can be achieved by mutations on either binding face alone. Complementary mutations in the same molecule on both binding faces generally are even more effective at disruption. For example the Large Domain double mutant D143N/A145R and Small Domain mutant Y87H effectively eliminate binding/signaling. In a homotrimeric complex of a mutant at a single face, each of the three receptor binding sites will be disrupted. In a heterotrimeric mixture of complementary mutations on different faces, as may be achieved by co-expression or exchange, there will be one receptor binding site disrupted on one face, one disrupted on two faces, and a third with no disruption.

In a preferred embodiment, the different protein members of the variant TNF-α library may be chemically synthesized. This is particularly useful when the designed proteins are short, preferably less than 150 amino acids in length, with less than 100 amino acids being preferred, and less than 50 amino acids being particularly preferred, although as is known in the art, longer proteins may be made chemically or enzymatically. See for example Wilken et al., Curr. Opin. Biotechnol. 9:412-26 (1998), hereby incorporated by reference.

In a preferred embodiment, particularly for longer proteins or proteins for which large samples are desired, the library sequences are used to create nucleic acids such as DNA which encode the member sequences and which may then be cloned into host cells, expressed and assayed, if desired. Thus, nucleic acids, and particularly DNA, may be made which encodes each member protein sequence. This is done using well known procedures. The choice of codons, suitable expression vectors and suitable host cells will vary depending on a number of factors, and may be easily optimized as needed.

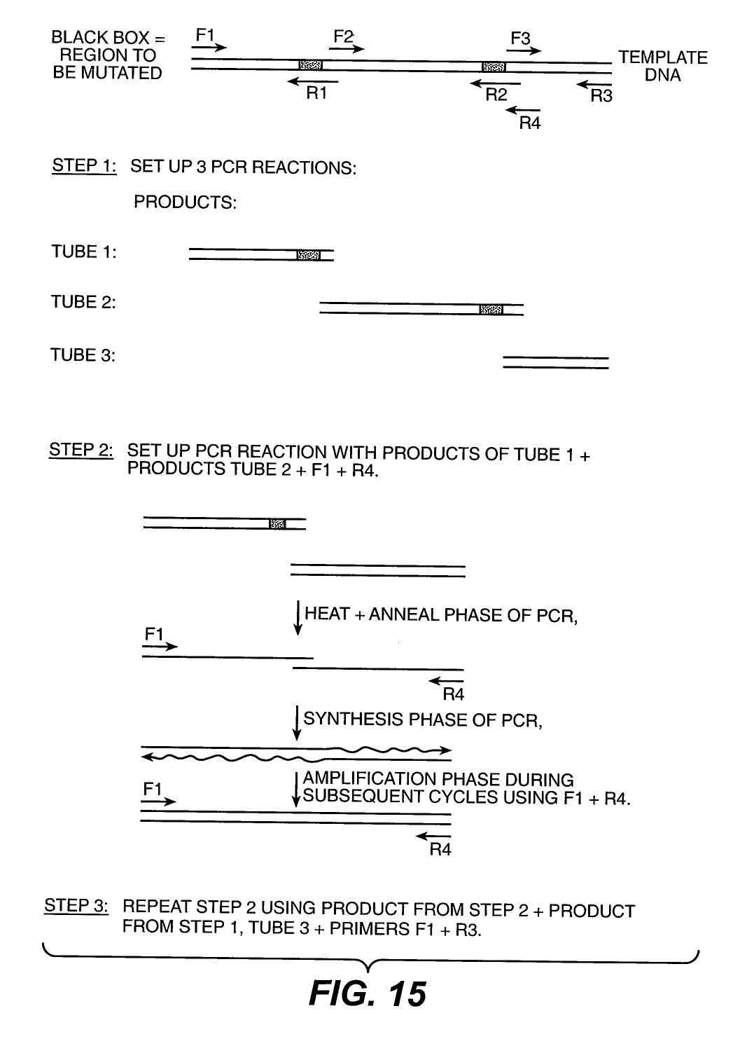

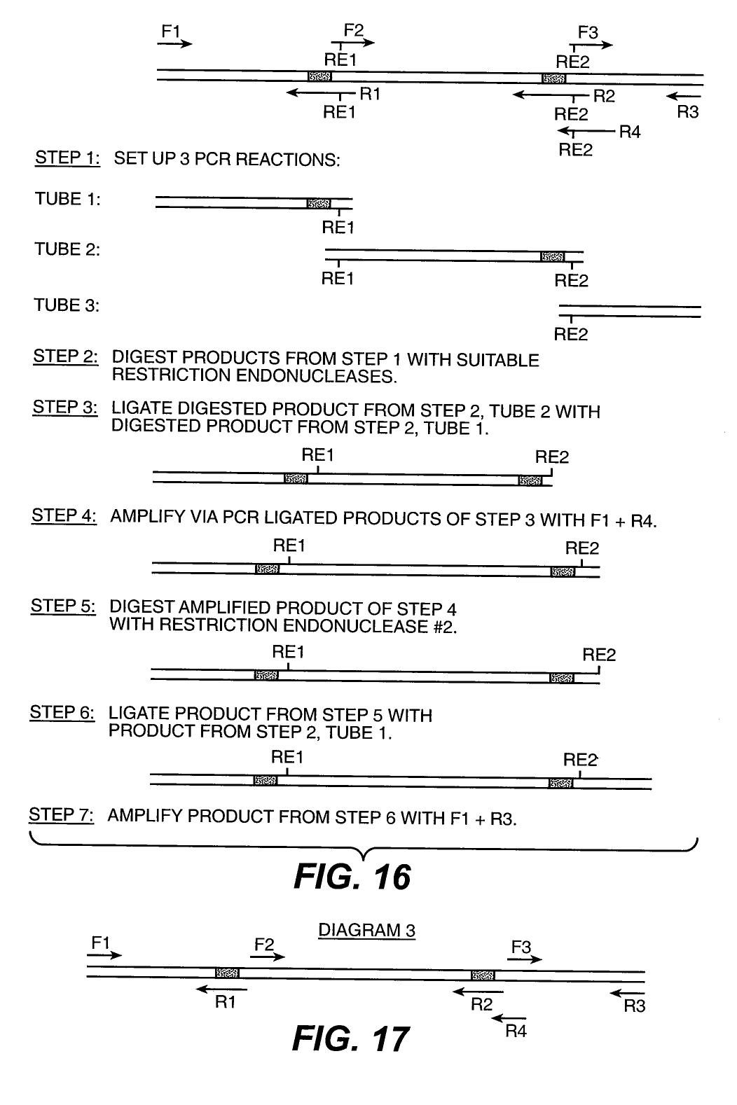

In a preferred embodiment, multiple PCR reactions with pooled oligonucleotides are done, as is generally depicted in the FIGS. 13-17. In this embodiment, overlapping oligonucleotides are synthesized which correspond to the full-length gene. Again, these oligonucleotides may represent all of the different amino acids at each variant position or subsets.

In a preferred embodiment, these oligonucleotides are pooled in equal proportions and multiple PCR reactions are performed to create full-length sequences containing the combinations of mutations defined by the library. In addition, this may be done using error-prone PCR methods. In a preferred embodiment, the different oligonucleotides are added in relative amounts corresponding to the probability distribution table. The multiple PCR reactions thus result in full length sequences with the desired combinations of mutations in the desired proportions. The total number of oligonucleotides needed is a function of the number of positions being mutated and the number of mutations being considered at these positions: (number of oligos for constant positions)+M1+M2+Mₙ=(total number of oligos required) where Mₙ is the number of mutations considered at position n in the sequence. The total number of oligonucleotides required increases when multiple mutable positions are encoded by a single oligonucleotide. The annealed regions are the ones that remain constant, i.e. have the sequence of the reference sequence.

Oligonucleotides with insertions or deletions of codons may be used to create a library expressing different length proteins. In particular computational sequence screening for insertions or deletions may result in secondary libraries defining different length proteins, which can be expressed by a library of pooled oligonucleotide of different lengths. In a preferred embodiment, the variant TNF-α library is done by shuffling the family (e.g. a set of variants); that is, some set of the top sequences (if a rank-ordered list is used) can be shuffled, either with or without error-prone PCR. “Shuffling” in this context means a recombination of related sequences, generally in a random way. It can include “shuffling” as defined and exemplified in U.S. Pat. Nos. 5,830,721; 5,811,238; 5,605,793; 5,837,458 and PCT US/19256, all of which are incorporated by reference. This set of sequences may also be an artificial set; for example, from a probability table (for example generated using SCMF) or a Monte Carlo set. Similarly, the “family” can be the top 10 and the bottom 10 sequences, the top 100 sequences, etc. This may also be done using error-prone PCR.

In a preferred embodiment, error-prone PCR is done to generate the variant TNF-α library. See U.S. Pat. Nos. 5,605,793, 5,811,238, and 5,830,721, all incorporated by reference. This may be done on the optimal sequence or on top members of the library, or some other artificial set or family. In this embodiment, the gene for the optimal sequence found in the computational screen of the primary library may be synthesized. Error-prone PCR is then performed on the optimal sequence gene in the presence of oligonucleotides that code for the mutations at the variant positions of the library (bias oligonucleotides). The addition of the oligonucleotides will create a bias favoring the incorporation of the mutations in the library. Alternatively, only oligonucleotides for certain mutations may be used to bias the library.

In a preferred embodiment, gene shuffling with error-prone PCR can be performed on the gene for the optimal sequence, in the presence of bias oligonucleotides, to create a DNA sequence library that reflects the proportion of the mutations found in the variant TNF-α library. The choice of the bias oligonucleotides can be done in a variety of ways; they can chosen on the basis of their frequency, i.e. oligonucleotides encoding high mutational frequency positions can be used: alternatively, oligonucleotides containing the most variable positions can be used, such that the diversity is increased; if the secondary library is ranked, some number of top scoring positions may be used to generate bias oligonucleotides; random positions may be chosen; a few top scoring and a few low scoring ones may be chosen; etc. What is important is to generate new sequences based on preferred variable positions and sequences.

In a preferred embodiment, PCR using a wild-type gene or other gene may be used, as is schematically depicted in the Figures. In this embodiment, a starting gene is used; generally, although this is not required, the gene is usually the wild-type gene. In some cases it may be the gene encoding the global optimized sequence, or any other sequence of the list, or a consensus sequence obtained e.g. from aligning homologous sequences from different organisms. In this embodiment, oligonucleotides are used that correspond to the variant positions and contain the different amino acids of the library. PCR is done using PCR primers at the termini, as is known in the art. This provides two benefits. First, this generally requires fewer oligonucleotides and may result in fewer errors. Second, it has experimental advantages in that if the wild-type gene is used, it need not be synthesized. In addition, there are several other techniques that may be used, as exemplified in FIGS. 13-17.

In a preferred embodiment, a variety of additional steps may be done to the variant TNF-α library; for example, further computational processing may occur, different variant TNF-α libraries can be recombined, or cutoffs from different libraries may be combined. In a preferred embodiment, a variant TNF-α library may be computationally remanipulated to form an additional variant TNF-α library (sometimes referred to as “tertiary libraries”). For example, any of the variant TNF-α library sequences may be chosen for a second round of PDA®, by freezing or fixing some or all of the changed positions in the first library. Alternatively, only changes seen in the last probability distribution table are allowed. Alternatively, the stringency of the probability table may be altered, either by increasing or decreasing the cutoff for inclusion. Similarly, the variant TNF-α library may be recombined experimentally after the first round; for example, the best gene/genes from the first screen may be taken and gene assembly redone (using techniques outlined below, multiple PCR, error-prone PCR, shuffling, etc.). Alternatively, the fragments from one or more good gene(s) to change probabilities at some positions.

In a preferred embodiment, a tertiary library may be generated from combining different variant TNF-α libraries. For example, a probability distribution table from a first variant TNF-α library may be generated and recombined, either computationally or experimentally, as outlined herein. A PDATM variant TNF-α library may be combined with a sequence alignment variant TNF-α library, and either recombined (again, computationally or experimentally) or just the cutoffs from each joined to make a new tertiary library. The top sequences from several libraries may be recombined. Sequences from the top of a library may be combined with sequences from the bottom of the library to more broadly sample sequence space, or only sequences distant from the top of the library may be combined. Variant TNF-α libraries that analyzed different parts of a protein may be combined to a tertiary library that treats the combined parts of the protein.

In a preferred embodiment, a tertiary library may be generated using correlations in a variant TNF-α library. That is, a residue at a first variable position may be correlated to a residue at second variable position (or correlated to residues at additional positions as well). For example, two variable positions may sterically or electrostatically interact, such that if the first residue is X, the second residue must be Y. This may be either a positive or negative correlation.

Using the nucleic acids of the present invention which encode a variant TNF-α protein, a variety of expression vectors are made. The expression vectors may be either self-replicating extrachromosomal vectors or vectors which integrate into a host genome. Generally, these expression vectors include transcriptional and translational regulatory nucleic acid operably linked to the nucleic acid encoding the variant TNF-α protein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation.

In a preferred embodiment, when the endogenous secretory sequence leads to a low level of secretion of the naturally occurring protein or of the variant TNF-α protein, a replacement of the naturally occurring secretory leader sequence is desired. In this embodiment, an unrelated secretory leader sequence is operably linked to a variant TNF-α encoding nucleic acid leading to increased protein secretion. Thus, any secretory leader sequence resulting in enhanced secretion of the variant TNF-α protein, when compared to the secretion of TNF-α and its secretory sequence, is desired. Suitable secretory leader sequences that lead to the secretion of a protein are known in the art. In another preferred embodiment, a secretory leader sequence of a naturally occurring protein or a protein is removed by techniques known in the art and subsequent expression results in intracellular accumulation of the recombinant protein.

Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice. The transcriptional and translational regulatory nucleic acid will generally be appropriate to the host cell used to express the fusion protein; for example, transcriptional and translational regulatory nucleic acid sequences from Bacillus are preferably used to express the fusion protein in Bacillus. Numerous types of appropriate expression vectors, and suitable regulatory sequences are known in the art for a variety of host cells.

In general, the transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences. Promoter sequences encode either constitutive or inducible promoters. The promoters may be either naturally occurring promoters or hybrid promoters. Hybrid promoters, which combine elements of more than one promoter, are also known in the art, and are useful in the present invention. In a preferred embodiment, the promoters are strong promoters, allowing high expression in cells, particularly mammalian cells, such as the CMV promoter, particularly in combination with a Tet regulatory element.

In addition, the expression vector may comprise additional elements. For example, the expression vector may have two replication systems, thus allowing it to be maintained in two organisms, for example in mammalian or insect cells for expression and in a prokaryotic host for cloning and amplification. Furthermore, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors are well known in the art.

In addition, in a preferred embodiment, the expression vector contains a selectable marker gene to allow the selection of transformed host cells. Selection genes are well known in the art and will vary with the host cell used. A preferred expression vector system is a retroviral vector system such as is generally described in PCT/US97/01019 and PCT/US97/01048, both of which are hereby incorporated by reference. In a preferred embodiment, the expression vector comprises the components described above and a gene encoding a variant TNF-α protein. As will be appreciated by those in the art, all combinations are possible and accordingly, as used herein, the combination of components, comprised by one or more vectors, which may be retroviral or not, is referred to herein as a “vector composition”.

The variant TNF-α nucleic acids are introduced into the cells either alone or in combination with an expression vector. By “introduced into ” or grammatical equivalents is meant that the nucleic acids enter the cells in a manner suitable for subsequent expression of the nucleic acid. The method of introduction is largely dictated by the targeted cell type, discussed below. Exemplary methods include CaPO4 precipitation, liposome fusion, lipofectin®, electroporation, viral infection, etc. The variant TNFa nucleic acids may stably integrate into the genome of the host cell (for example, with retroviral introduction, outlined below), or may exist either transiently or stably in the cytoplasm (i.e. through the use of traditional plasmids, utilizing standard regulatory sequences, selection markers, etc.).

The variant TNF-α proteins of the present invention are produced by culturing a host cell transformed with an expression vector containing nucleic acid encoding a variant TNF-α protein, under the appropriate conditions to induce or cause expression of the variant TNF-α protein. The conditions appropriate for variant TNF-α protein expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield. Appropriate host cells include yeast, bacteria, archaebacteria, fungi, and insect and animal cells, including mammalian cells. Of interest are Drosophila melangaster cells, Saccharomyces cerevisiae and other yeasts, E. coli, Bacillus subtilis, SF9 cells, C129 cells, 293 cells, Neurospora, BHK, CHO, COS, Pichia pastoris, etc.

In a preferred embodiment, the variant TNF-α proteins are expressed in mammalian cells. Mammalian expression systems are also known in the art, and include retroviral systems. A mammalian promoter is any DNA sequence capable of binding mammalian RNA polymerase and initiating the downstream (3′) transcription of a coding sequence for the fusion protein into mRNA. A promoter will have a transcription initiating region, which is usually placed proximal to the 5′ end of the coding sequence, and a TATA box, using a located 25-30 base pairs upstream of the transcription initiation site. The TATA box is thought to direct RNA polymerase II to begin RNA synthesis at the correct site. A mammalian promoter will also contain an upstream promoter element (enhancer element), typically located within 100 to 200 base pairs upstream of the TATA box. An upstream promoter element determines the rate at which transcription is initiated and can act in either orientation. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter. Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenylation signals include those derived from SV40.

The methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei. As outlined herein, a particularly preferred method utilizes retroviral infection, as outlined in PCT US97/01019, incorporated by reference.

As will be appreciated by those in the art, the type of mammalian cells used in the present invention can vary widely. Basically, any mammalian cells may be used, with mouse, rat, primate and human cells being particularly preferred, although as will be appreciated by those in the art, modifications of the system by pseudotyping allows all eukaryotic cells to be used, preferably higher eukaryotes. As is more fully described below, a screen will be set up such that the cells exhibit a selectable phenotype in the presence of a bioactive peptide. As is more fully described below, cell types implicated in a wide variety of disease conditions are particularly useful, so long as a suitable screen may be designed to allow the selection of cells that exhibit an altered phenotype as a consequence of the presence of a peptide within the cell.

Accordingly, suitable cell types include, but are not limited to, tumor cells of all types (particularly melanoma, myeloid leukemia, carcinomas of the lung, breast, ovaries, colon, kidney, prostate, pancreas and testes), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as haemopoietic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, and adipocytes. Suitable cells also include known research cells, including, but not limited to, Jurkat T cells, NIH3T3 cells, CHO, COS, etc. See the ATCC cell line catalog, hereby incorporated by reference.

In one embodiment, the cells may be additionally genetically engineered, that is, contain exogenous nucleic acid other than the variant TNF-α nucleic acid. In a preferred embodiment, the variant TNF-α proteins are expressed in bacterial systems. Bacterial expression systems are well known in the art. A suitable bacterial promoter is any nucleic acid sequence capable of binding bacterial RNA polymerase and initiating the downstream (3′) transcription of the coding sequence of the variant TNF-α protein into mRNA. A bacterial promoter has a transcription initiation region which is usually placed proximal to the 5′ end of the coding sequence. This transcription initiation region typically includes an RNA polymerase binding site and a transcription initiation site. Sequences encoding metabolic pathway enzymes provide particularly useful promoter sequences. Examples include promoter sequences derived from sugar metabolizing enzymes, such as galactose, lactose and maltose, and sequences derived from biosynthetic enzymes such as tryptophan. Promoters from bacteriophage may also be used and are known in the art. In addition, synthetic promoters and hybrid promoters are also useful; for example, the tac promoter is a hybrid of the trp and lac promoter sequences. Furthermore, a bacterial promoter may include naturally occurring promoters of non-bacterial origin that have the ability to bind bacterial RNA polymerase and initiate transcription. In addition to a functioning promoter sequence, an efficient ribosome binding site is desirable. In E. coli, the ribosome binding site is called the Shine-Delgarno (SD) sequence and includes an initiation codon and a sequence 3-9 nucleotides in length located 3-11 nucleotides upstream of the initiation codon.

The expression vector may also include a signal peptide sequence that provides for secretion of the variant TNF-α protein in bacteria. The signal sequence typically encodes a signal peptide comprised of hydrophobic amino acids which direct the secretion of the protein from the cell, as is well known in the art. The protein is either secreted into the growth media (gram-positive bacteria) or into the periplasmic space, located between the inner and outer membrane of the cell (gram-negative bacteria). For expression in bacteria, usually bacterial secretory leader sequences, operably linked to a variant TNF-α encoding nucleic acid, are preferred. The bacterial expression vector may also include a selectable marker gene to allow for the selection of bacterial strains that have been transformed. Suitable selection genes include genes which render the bacteria resistant to drugs such as ampicillin, chloramphenicol, erythromycin, kanamycin, neomycin and tetracycline. Selectable markers also include biosynthetic genes, such as those in the histidine, tryptophan and leucine biosynthetic pathways. These components are assembled into expression vectors. Expression vectors for bacteria are well known in the art, and include vectors for Bacillus subtilis, E. coli, Streptococcus cremoris, and Streptococcus lividans, among others. The bacterial expression vectors are transformed into bacterial host cells using techniques well known in the art, such as calcium chloride treatment, electroporation, and others. In one embodiment, variant TNF-α proteins are produced in insect cells. Expression vectors for the transformation of insect cells, and in particular, baculovirus-based expression vectors, are well known in the art. In a preferred embodiment, variant TNF-α protein is produced in yeast cells. Yeast expression systems are well known in the art, and include expression vectors for Saccharomyces cerevisiae, Candida albicans and C. maltosa, Hansenula polymorpha, Kluyveromyces fragilis and K. lactis, Pichia guillerimondii and P. pastoris, Schizosaccharomyces pombe, and Yarrowia lipolytica. Preferred promoter sequences for expression in yeast include the inducible GAL1, 10 promoter, the promoters from alcohol dehydrogenase, enolase, glucokinase, glucose-6-phosphate isomerase, glyceraldehyde-3-phosphate-dehydrogenase, hexokinase, phosphofructokinase, 3-phosphoglycerate mutase, pyruvate kinase, and the acid phosphatase gene. Yeast selectable markers include ADE2, HIS4, LEU2, TRP1, and ALG7, which confers resistance to tunicamycin; the neomycin phosphotransferase gene, which confers resistance to G418; and the CUP1 gene, which allows yeast to grow in the presence of copper ions.

In an alternative embodiment, modified TNF variants are covalently coupled to at least one additional TNF variant via a linker to improve the dominant negative action of the modified domains. A number of strategies may be used to covalently link modified receptor domains together. These include, but are not limited to, linkers, such as polypeptide linkages between N- and C-termini of two domains, linkage via a disulfide bond between monomers, and linkage via chemical cross-linking reagents. Alternatively, the N- and C-termini may be covalently joined by deletion of portions of the N- and/or C-termini and linking the remaining fragments via a linker or linking the fragments directly.

By “linker”, “linker sequence”, “spacer”, “tethering sequence” or grammatical equivalents thereof, is meant a molecule or group of molecules (such as a monomer or polymer) that connects two molecules and often serves to place the two molecules in a preferred configuration. In one aspect of this embodiment, the linker is a peptide bond. Choosing a suitable linker for a specific case where two polypeptide chains are to be connected depends on various parameters, e.g., the nature of the two polypeptide chains (e.g., whether they naturally oligomerize (e.g., form a dimer or not), the distance between the N- and the C-termini to be connected if known from three-dimensional structure determination, and/or the stability of the linker towards proteolysis and oxidation. Furthermore, the linker may contain amino acid residues that provide flexibility. Thus, the linker peptide may predominantly include the following amino acid residues: Gly, Ser, Ala, or Thr. These linked TNF-α proteins have constrained hydrodynamic properties, that is, they form constitutive dimers) and thus efficiently interact with other naturally occurring TNF-α proteins to form a dominant negative heterotrimer.

The linker peptide should have a length that is adequate to link two TNF variant monomers in such a way that they assume the correct conformation relative to one another so that they retain the desired activity as antagonists of the TNF receptor. Suitable lengths for this purpose include at least one and not more than 30 amino acid residues. Preferably, the linker is from about 1 to 30 amino acids in length, with linkers of 1, 2, 3, 4, 5, 6, 7, 8, 9, 10, 11, 12, 13, 14, 15, 16, 17, 18 19 and 20 amino acids in length being preferred. See also WO 01/25277, incorporated by reference in its entirety.

In addition, the amino acid residues selected for inclusion in the linker peptide should exhibit properties that do not interfere significantly with the activity of the polypeptide. Thus, the linker peptide on the whole should not exhibit a charge that would be inconsistent with the activity of the polypeptide, or interfere with internal folding, or form bonds or other interactions with amino acid residues in one or more of the monomers that would seriously impede the binding of receptor monomer domains. Useful linkers include glycine-serine polymers (including, for example, (GS)n, (GSGGS)n (SEQ ID NO. 20)(GGGGS)n (SEQ ID NO. 21) and (GGGS)n (SEQ ID NO. 22), where n is an integer of at least one), glycine-alanine polymers, alanine-serine polymers, and other flexible linkers such as the tether for the shaker potassium channel, and a large variety of other flexible linkers, as will be appreciated by those in the art. Glycine-serine polymers are preferred since both of these amino acids are relatively unstructured, and therefore may be able to serve as a neutral tether between components. Secondly, serine is hydrophilic and therefore able to solubilize what could be a globular glycine chain. Third, similar chains have been shown to be effective in joining subunits of recombinant proteins such as single chain antibodies. Suitable linkers may also be identified by screening databases of known three-dimensional structures for naturally occurring motifs that can bridge the gap between two polypeptide chains. Another way of obtaining a suitable linker is by optimizing a simple linker, e.g., (Gly4Ser)n, through random mutagenesis. Alternatively, once a suitable polypeptide linker is defined, additional linker polypeptides can be created by application of PDA® technology to select amino acids that more optimally interact with the domains being linked. Other types of linkers that may be used in the present invention include artificial polypeptide linkers and inteins. In another preferred embodiment, disulfide bonds are designed to link the two receptor monomers at inter-monomer contact sites. In one aspect of this embodiment the two receptors are linked at distances <5 Angstroms. In addition, the variant TNF-α polypeptides of the invention may be further fused to other proteins, if desired, for example to increase expression or stabilize the protein.

In one embodiment, the variant TNF-α nucleic acids, proteins and antibodies of the invention are labeled with a label other than the scaffold. By “labeled” herein is meant that a compound has at least one element, isotope or chemical compound attached to enable the detection of the compound. In general, labels fall into three classes: a) isotopic labels, which may be radioactive or heavy isotopes; b) immune labels, which may be antibodies or antigens; and c) colored or fluorescent dyes. The labels may be incorporated into the compound at any position.

Once made, the variant TNF-α proteins may be covalently modified. Covalent and non-covalent modifications of the protein are thus included within the scope of the present invention. Such modifications may be introduced into a variant TNF-α polypeptide by reacting targeted amino acid residues of the polypeptide with an organic derivatizing agent that is capable of reacting with selected side chains or terminal residues. One type of covalent modification includes reacting targeted amino acid residues of a variant TNF-α polypeptide with an organic derivatizing agent that is capable of reacting with selected side chains or the N- or C-terminal residues of a variant TNF-α polypeptide. Derivatization with bifunctional agents is useful, for instance, for cross linking a variant TNF-α protein to a water-insoluble support matrix or surface for use in the method for purifying anti-variant TNF-α antibodies or screening assays, as is more fully described below. Commonly used cross linking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio] propioimidate. Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the “-amino groups of lysine, arginine, and histidine side chains [T. E. Creighton, Proteins: Structure and Molecular Properties, W.H. Freeman & Co., San Francisco, pp. 79-86 (1983), incorporated by reference,] acetylation of the N-terminal amine, and amidation of any C-terminal carboxyl group.

Another type of covalent modification of the variant TNF-α polypeptide included within the scope of this invention comprises altering the native glycosylation pattern of the polypeptide. “Altering the native glycosylation pattern” is intended for purposes herein to mean deleting one or more carbohydrate moieties found in native sequence variant TNF-α polypeptide, and/or adding one or more glycosylation sites that are not present in the native sequence variant TNF-α polypeptide. Addition of glycosylation sites to variant TNF-α polypeptides may be accomplished by altering the amino acid sequence thereof. The alteration may be made, for example, by the addition of, or substitution by, one or more serine or threonine residues to the native sequence or variant TNF-α polypeptide (for O-linked glycosylation sites). The variant TNF-α amino acid sequence may optionally be altered through changes at the DNA level, particularly by mutating the DNA encoding the variant TNF-α polypeptide at preselected bases such that codons are generated that will translate into the desired amino acids.

Addition of N-linked glycosylation sites to variant TNF-α polypeptides may be accomplished by altering the amino acid sequence thereof. The alteration may be made, for example, by the addition of, or substitution by, one or more asparagine residues to the native sequence or variant TNF-α polypeptide. The modification may be made for example by the incorporation of a canonical N-linked glycosylation site, including but not limited to, N—X—Y, where X is any amino acid except for proline and Y is preferably threonine, serine or cysteine. Another means of increasing the number of carbohydrate moieties on the variant TNF-α polypeptide is by chemical or enzymatic coupling of glycosides to the polypeptide. Such methods are described in the art, e.g., in WO 87/05330 published 11 Sep. 1987, and in Aplin and Wriston, CRC Crit. Rev. Biochem., pp. 259-306 (1981), incorporated by reference. Removal of carbohydrate moieties present on the variant TNF-α polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al, Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987), incorporated by reference. Such derivati2ed moieties may improve the solubility, absorption, and permeability across the blood brain barrier biological half-life, and the like. Such moieties or modifications of variant TNF-α polypeptides may alternatively eliminate or attenuate any possible undesirable side effect of the protein and the like. Moieties capable of mediating such effects are disclosed, for example, in Remington's Pharmaceutical Sciences, 16th ed., Mack Publishing Co., Easton, Pa. (1980), incorporated by reference.

Another type of covalent modification of variant TNF-α comprises linking the variant TNF-α polypeptide to one of a variety of nonproteinaceous polymers, e.g., polyethylene glycol (“PEG”), polypropylene glycol, or polyoxyalkylenes, in the manner set forth in U.S. Pat. No. 4,640,835; 4,496,689; 4,301,144; 4,670,417; 4,791,192 or 4,179,337, each of which is incorporated by reference in its entirety. These nonproteinaceous polymers may also be used to enhance the variant TNF-α's ability to disrupt receptor binding, and/or in vivo stability. In another preferred embodiment, cysteines are designed into variant or wild type TNF-α in order to incorporate (a) labeling sites for characterization and (b) incorporate PEGylation sites. For example, labels that may be used are well known in the art and include but are not limited to biotin, tag and fluorescent labels (e.g. fluorescein). These labels may be used in various assays as are also well known in the art to achieve characterization. A variety of coupling chemistries may be used to achieve PEGylation, as is well known in the art. Examples include but are not limited to, the technologies of Shearwater and Enzon, which allow modification at primary amines, including but not limited to, lysine groups and the N-terminus. See, Kinstler et al., Advanced Drug Deliveries Reviews, 54, 477-485 (2002) and M J Roberts et al., Advanced Drug Delivery Reviews, 54, 459-476 (2002), both hereby incorporated by reference.

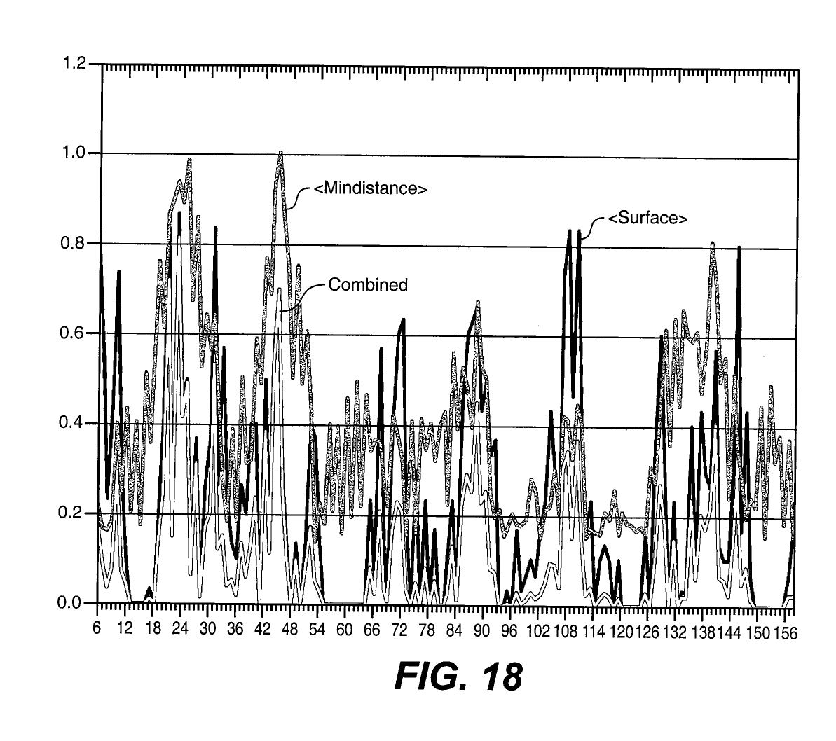

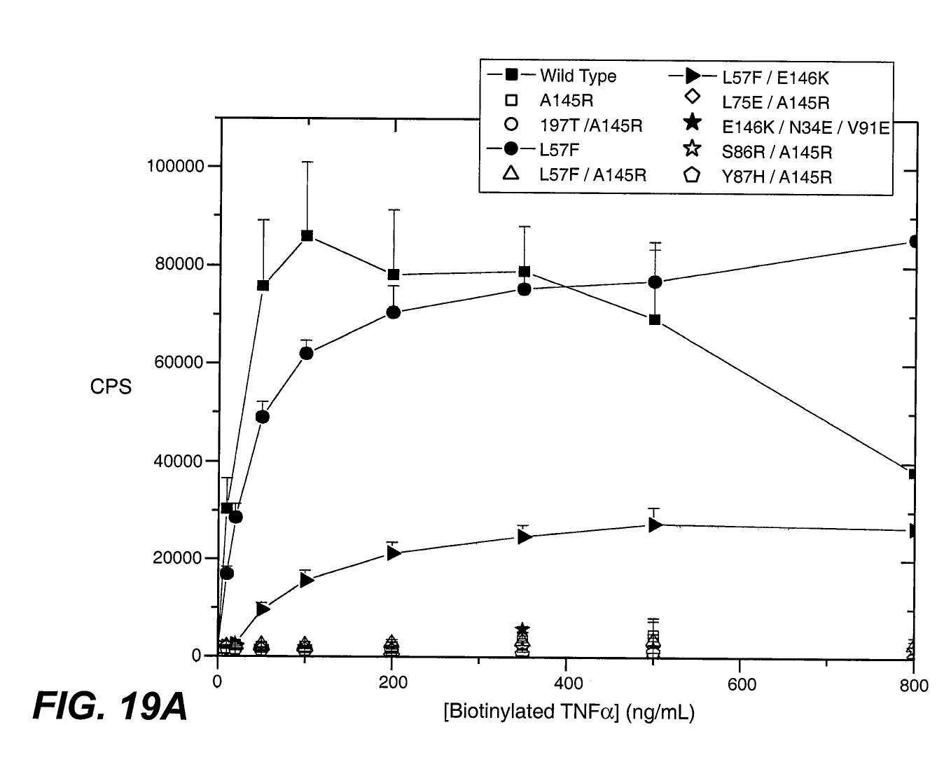

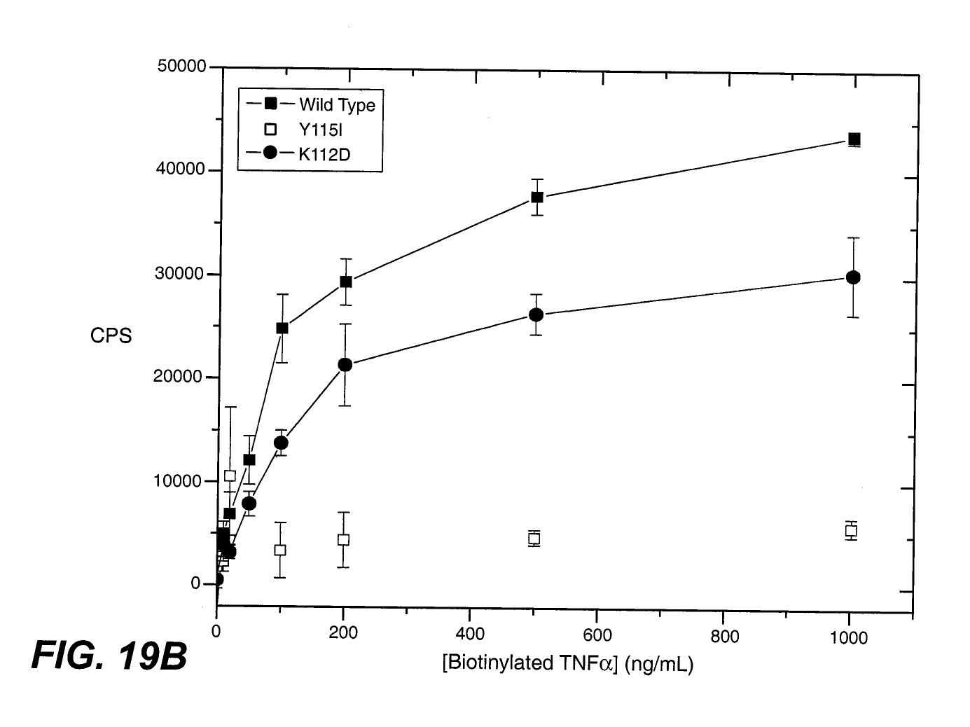

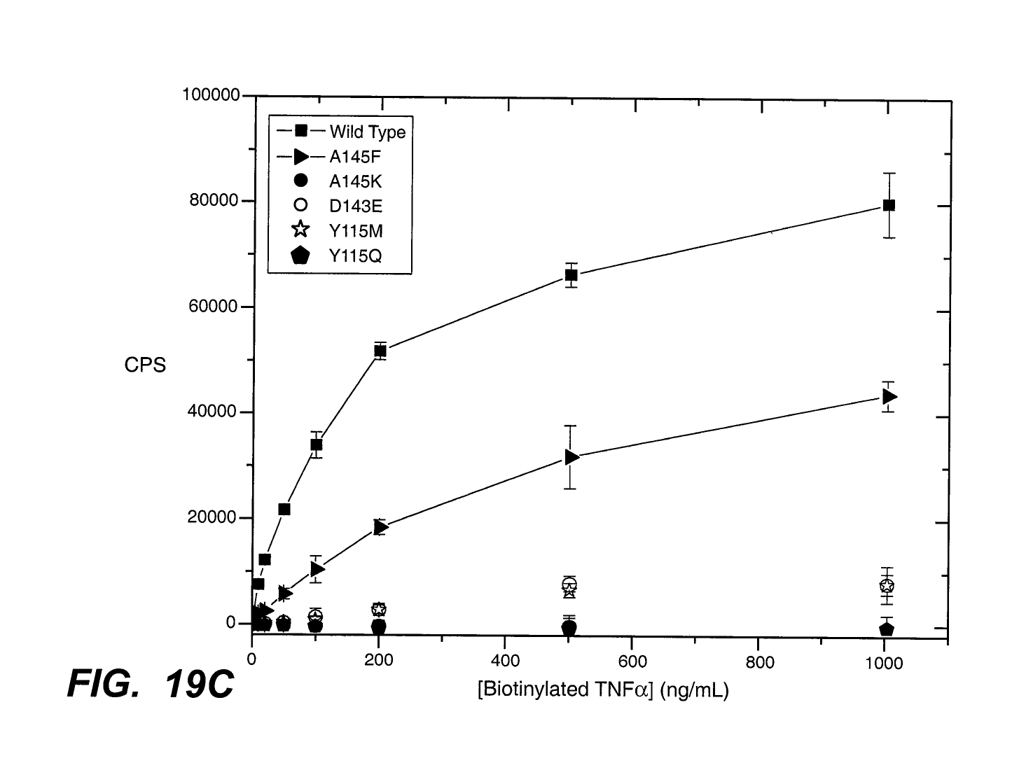

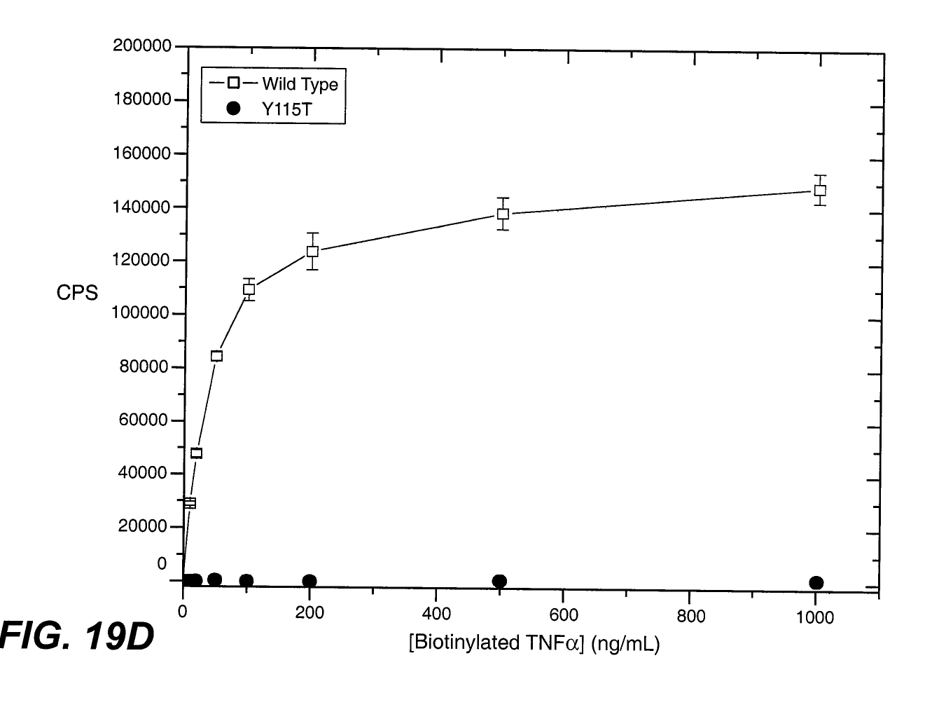

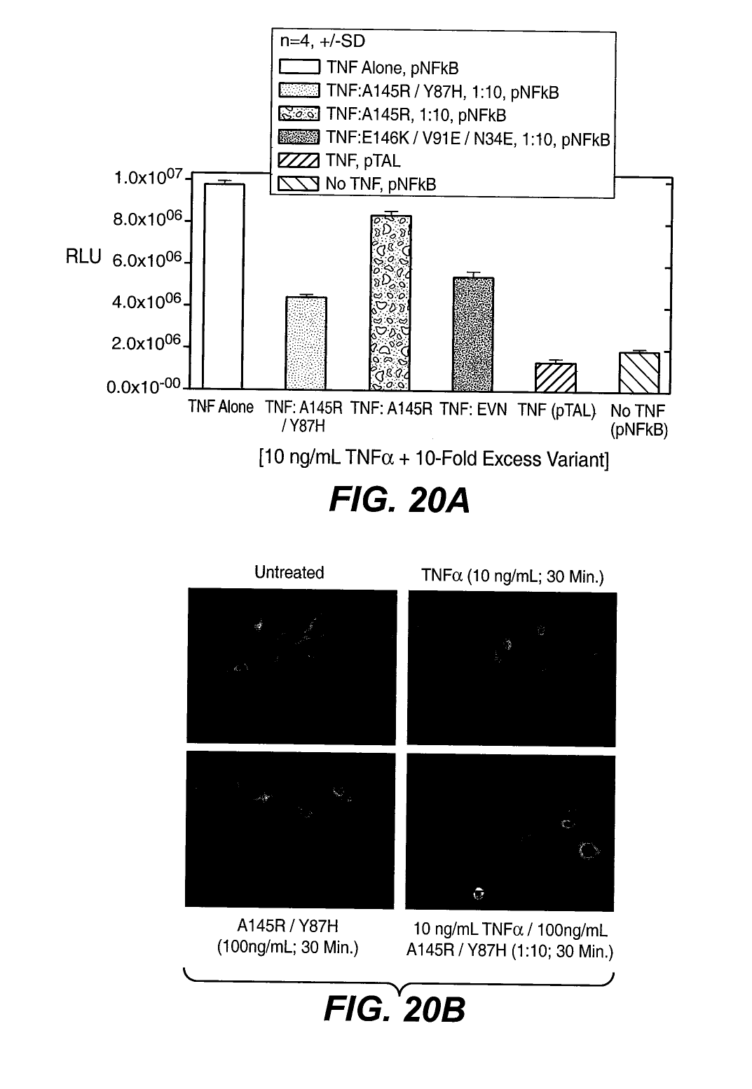

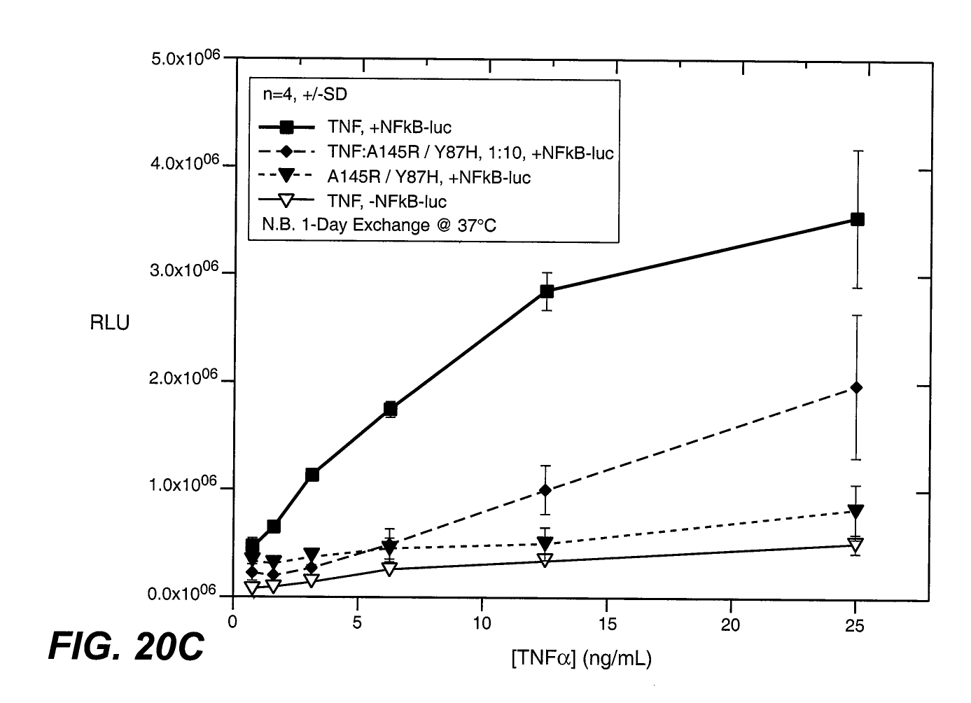

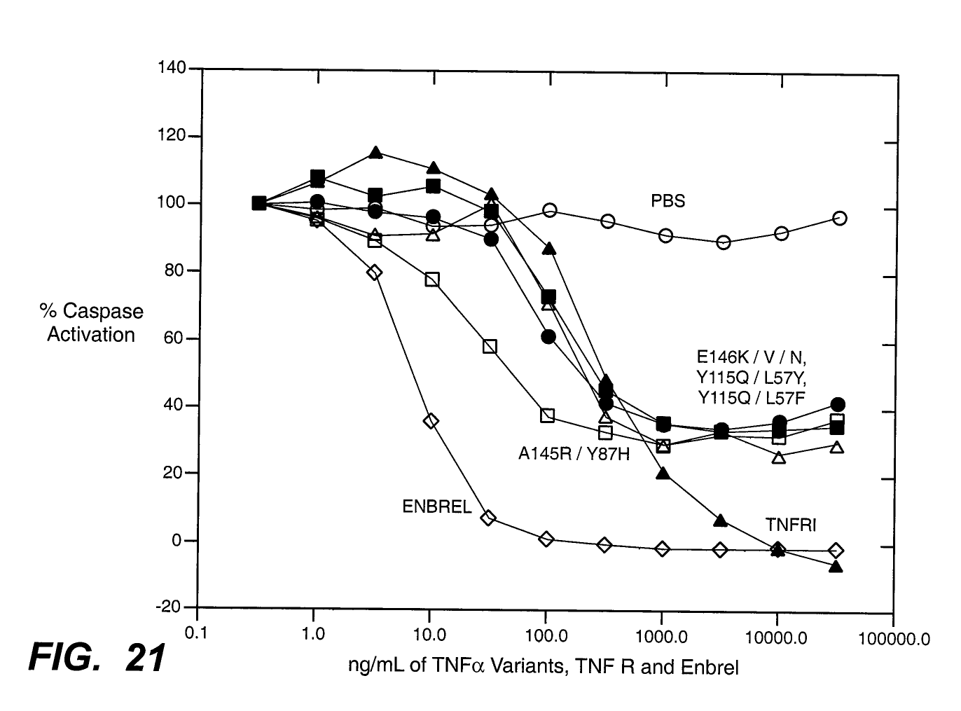

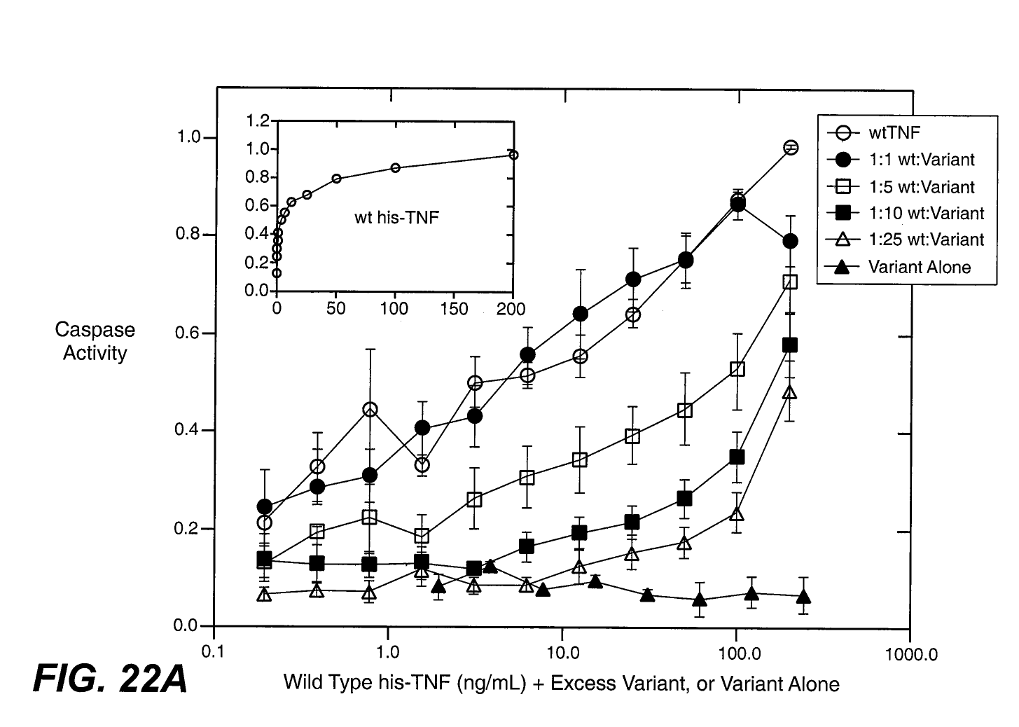

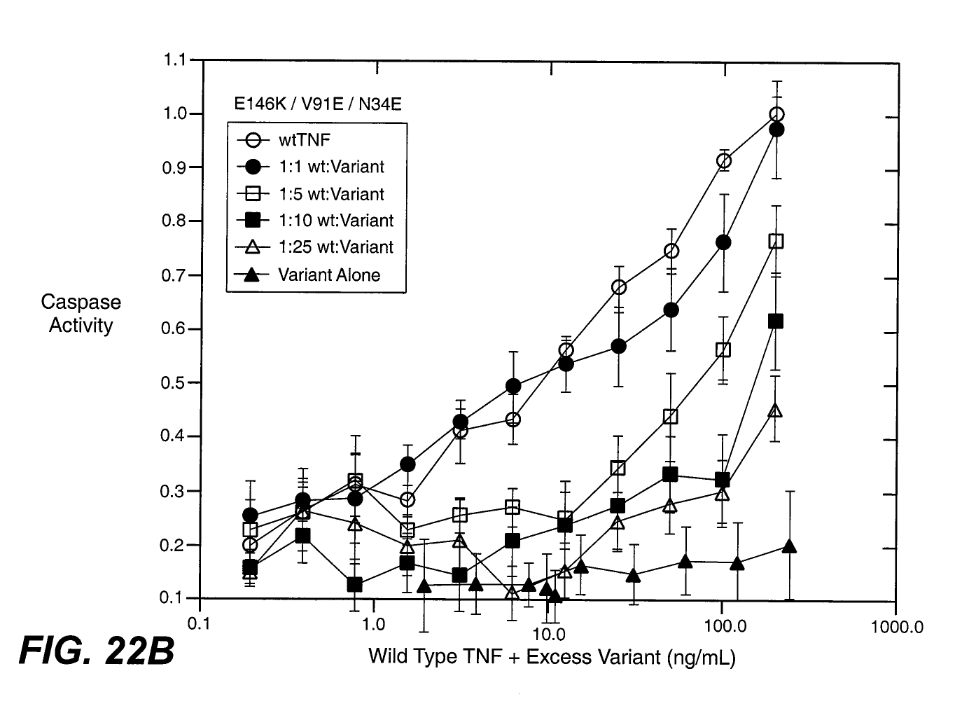

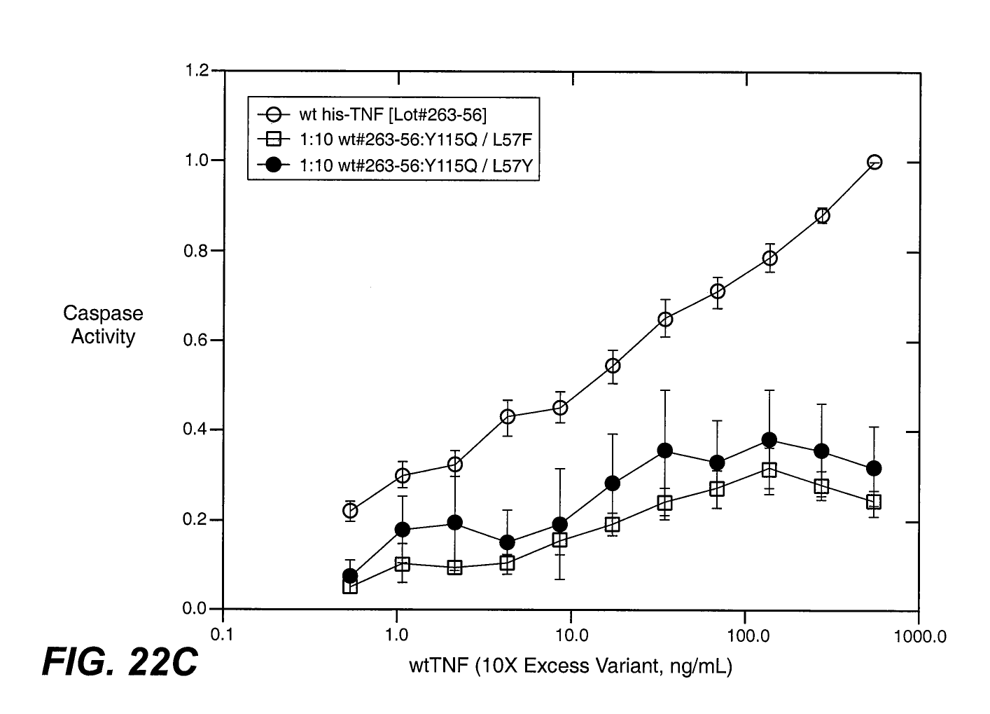

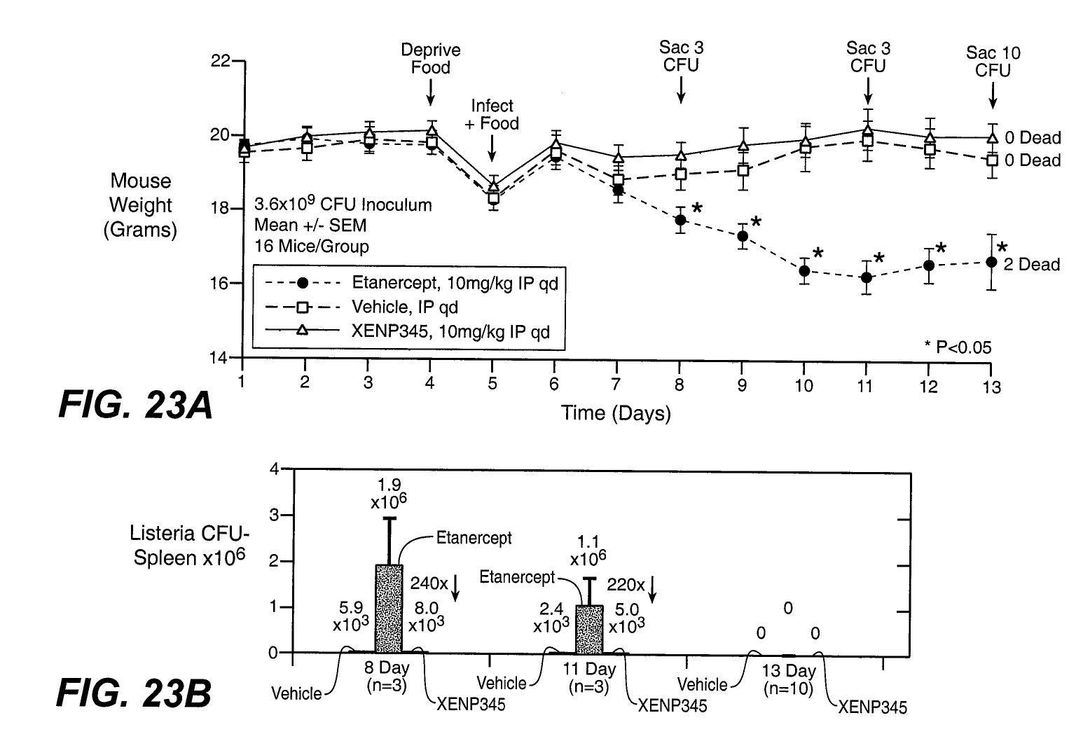

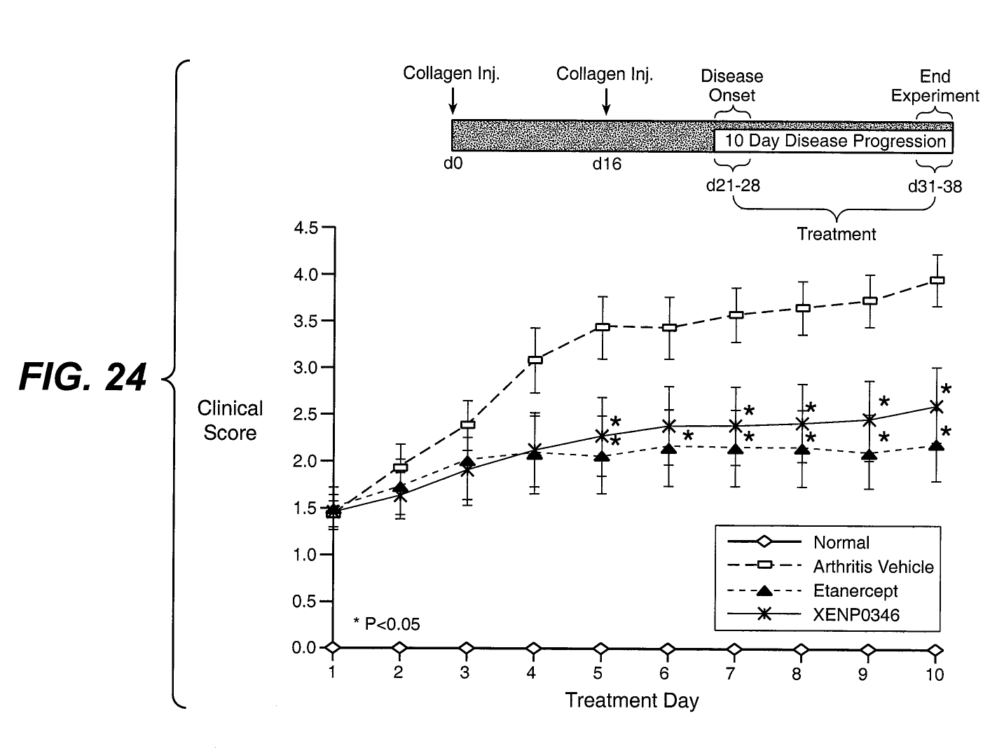

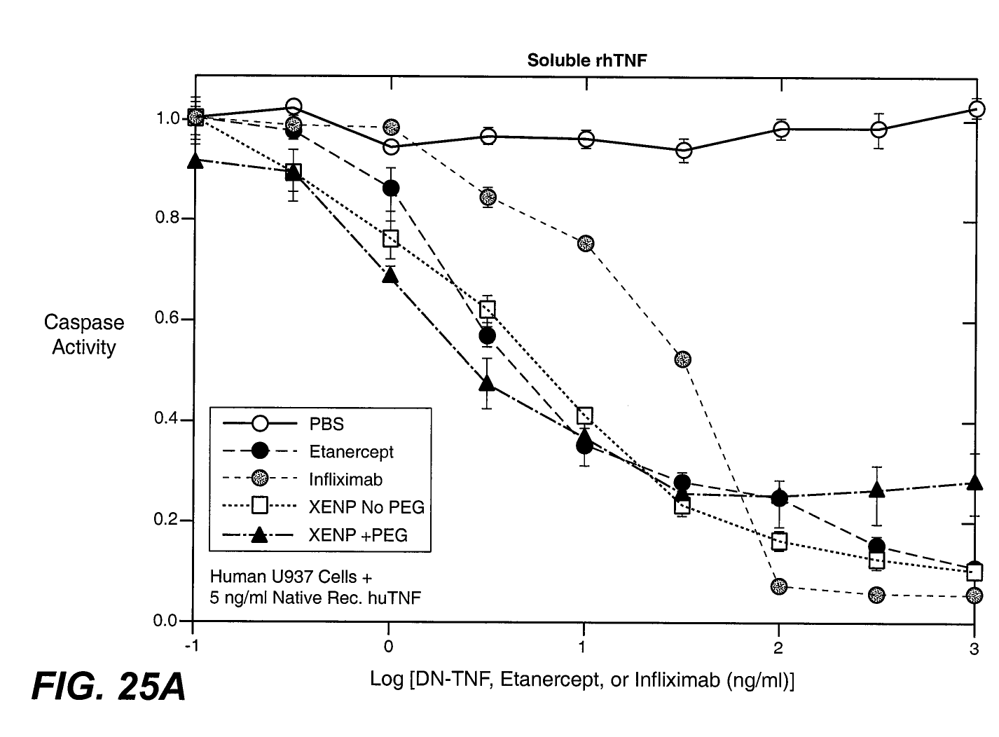

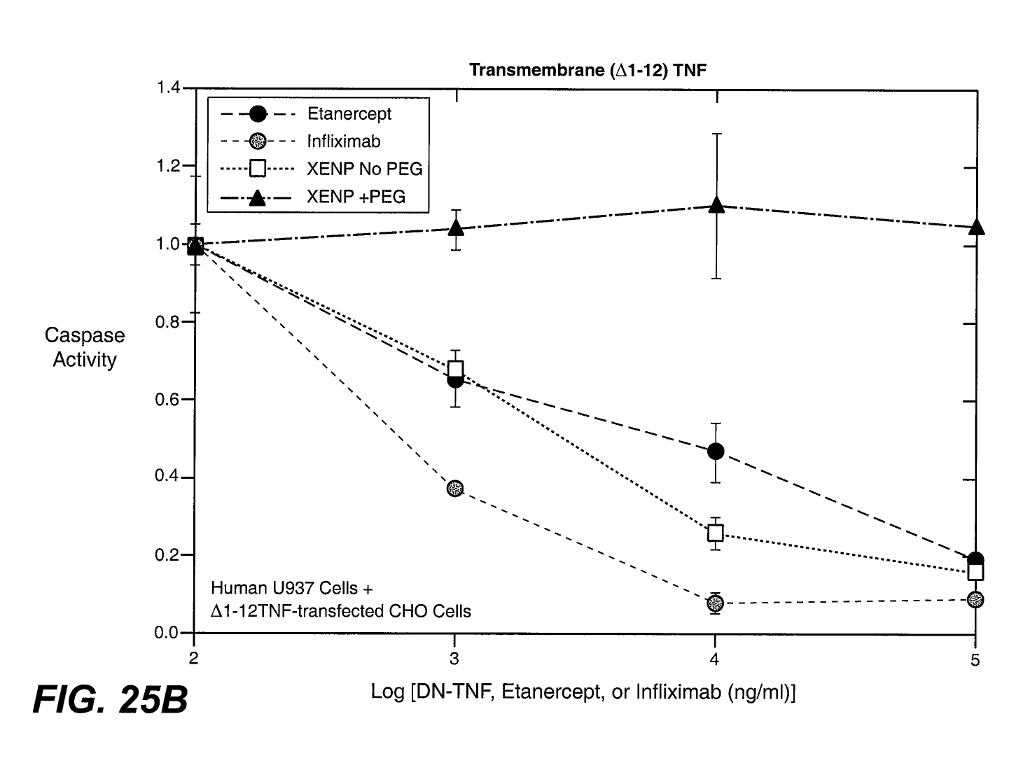

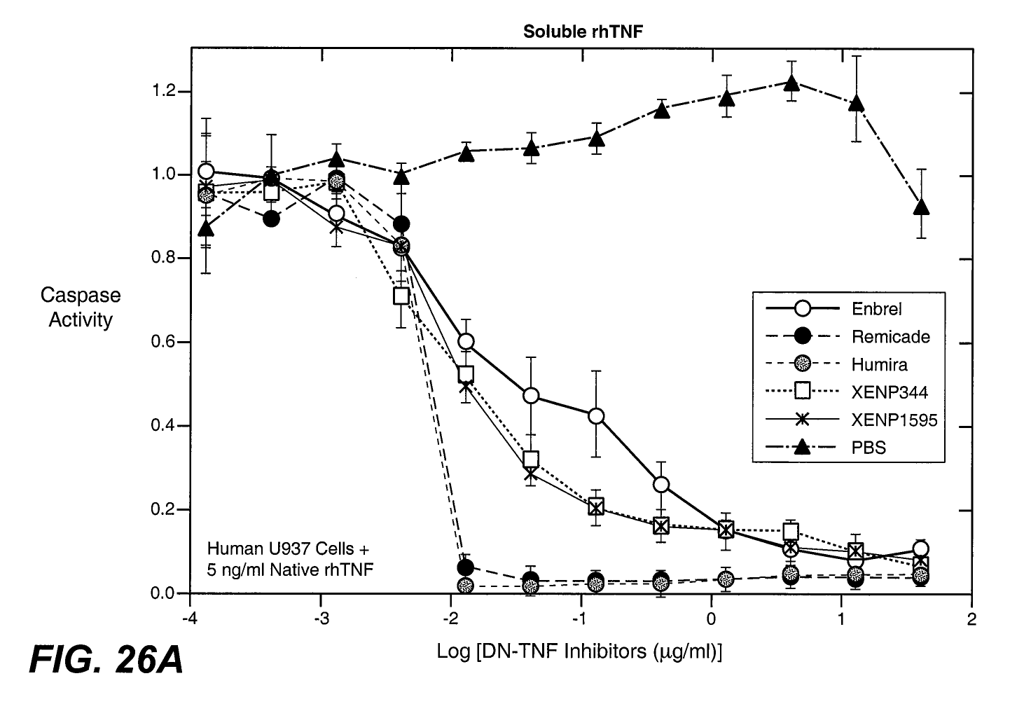

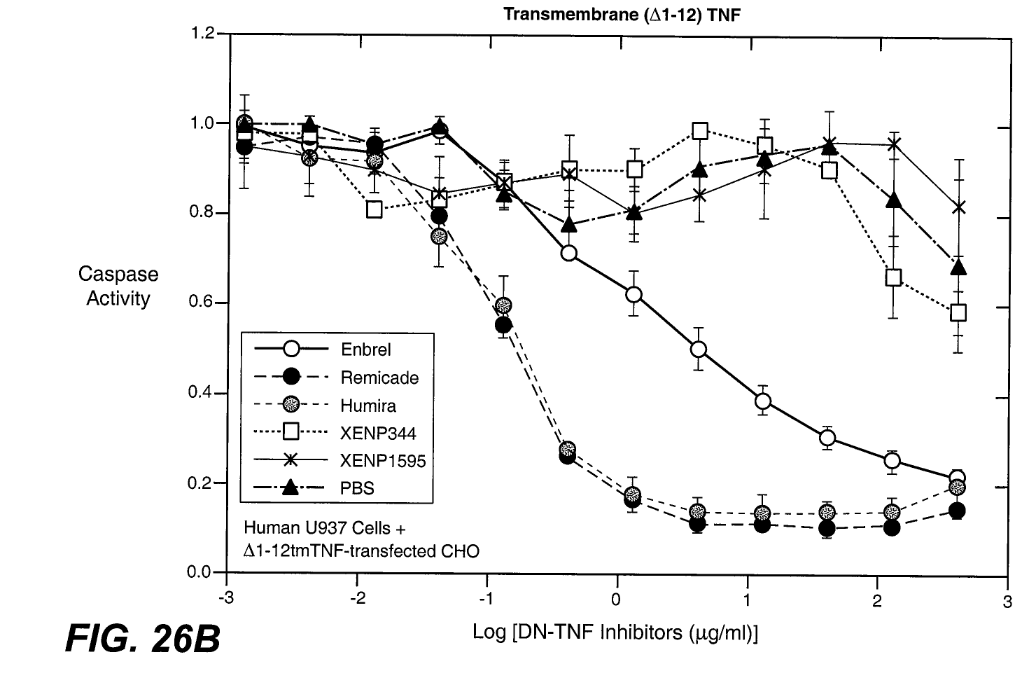

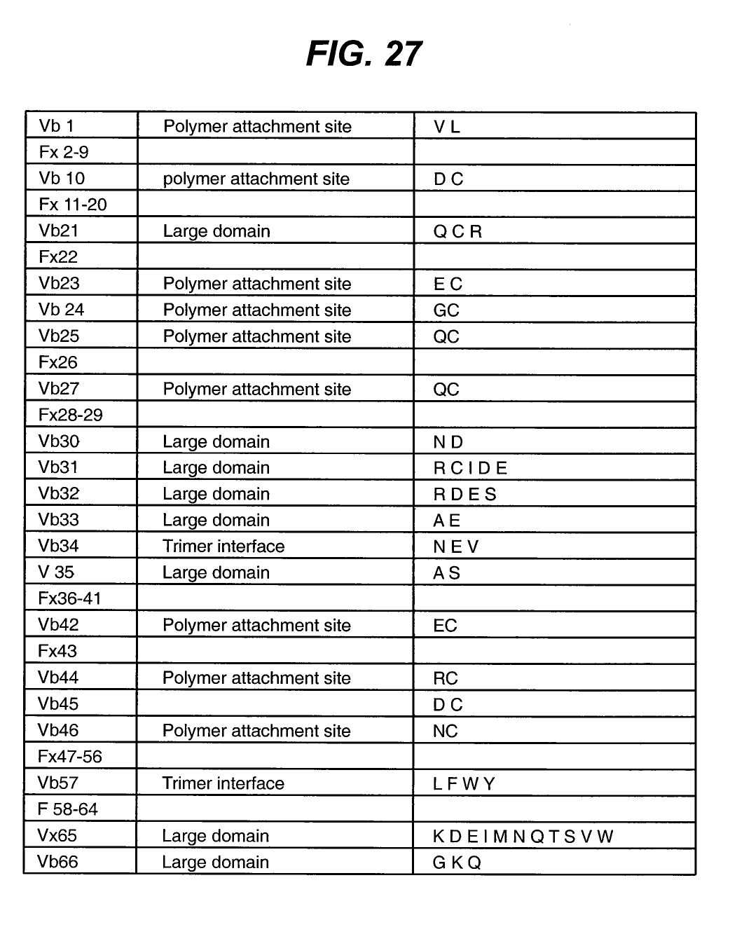

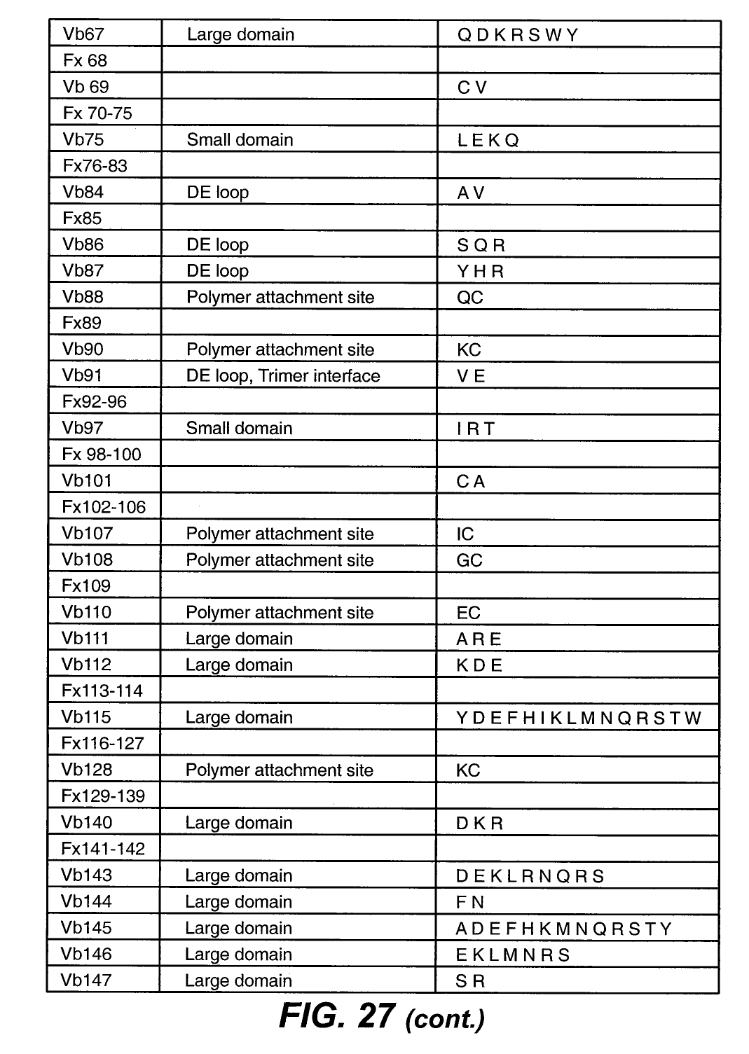

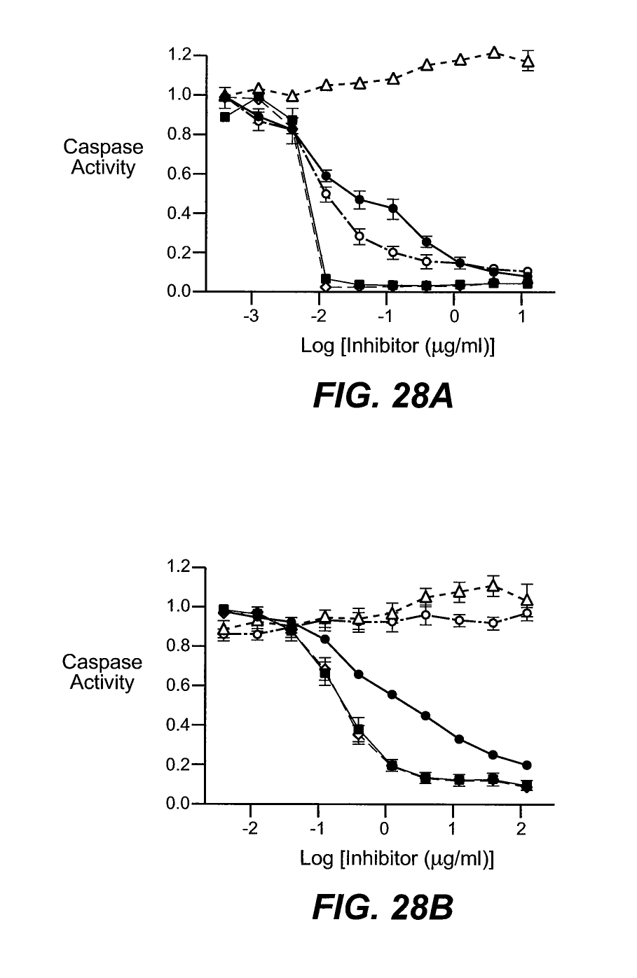

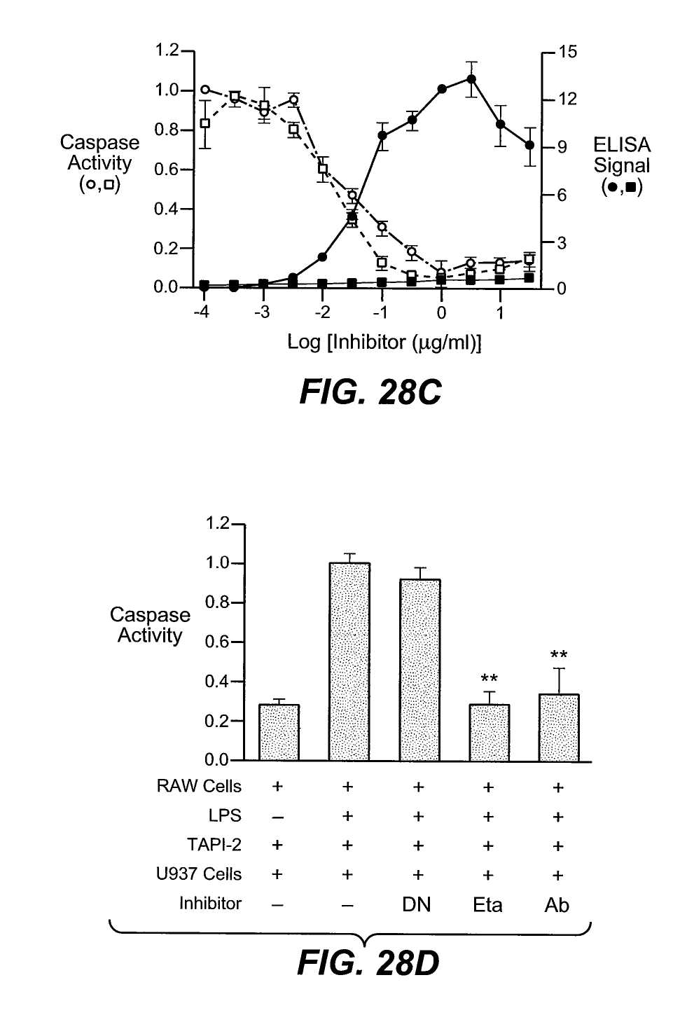

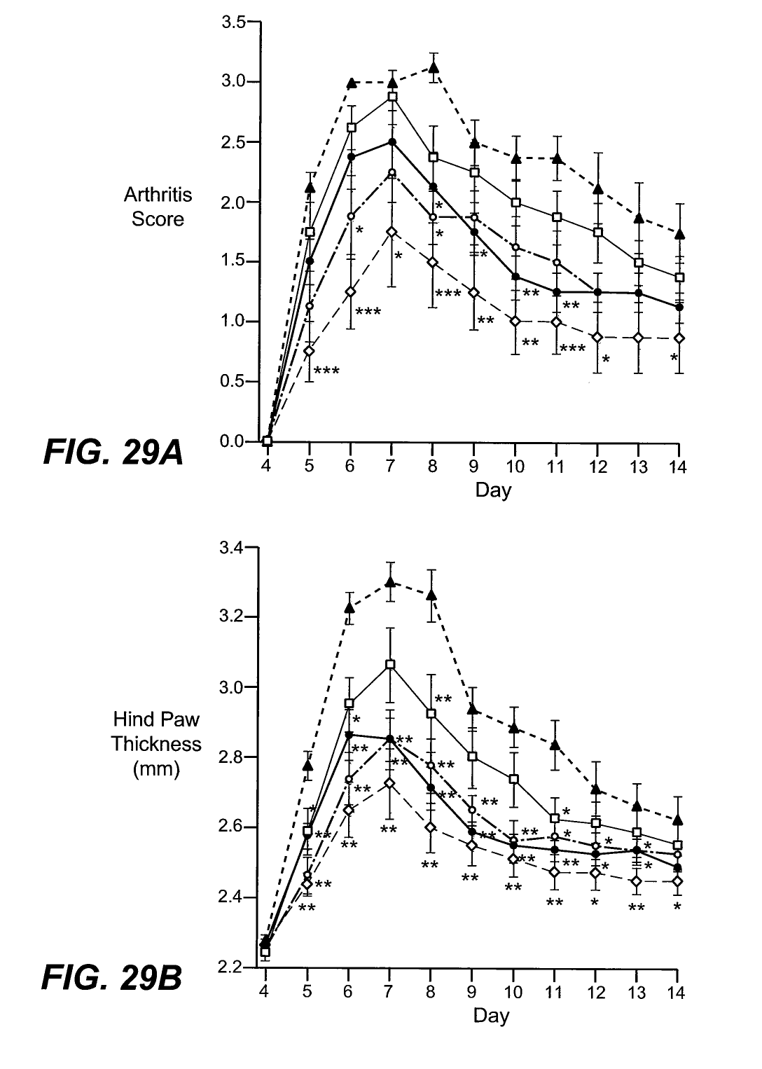

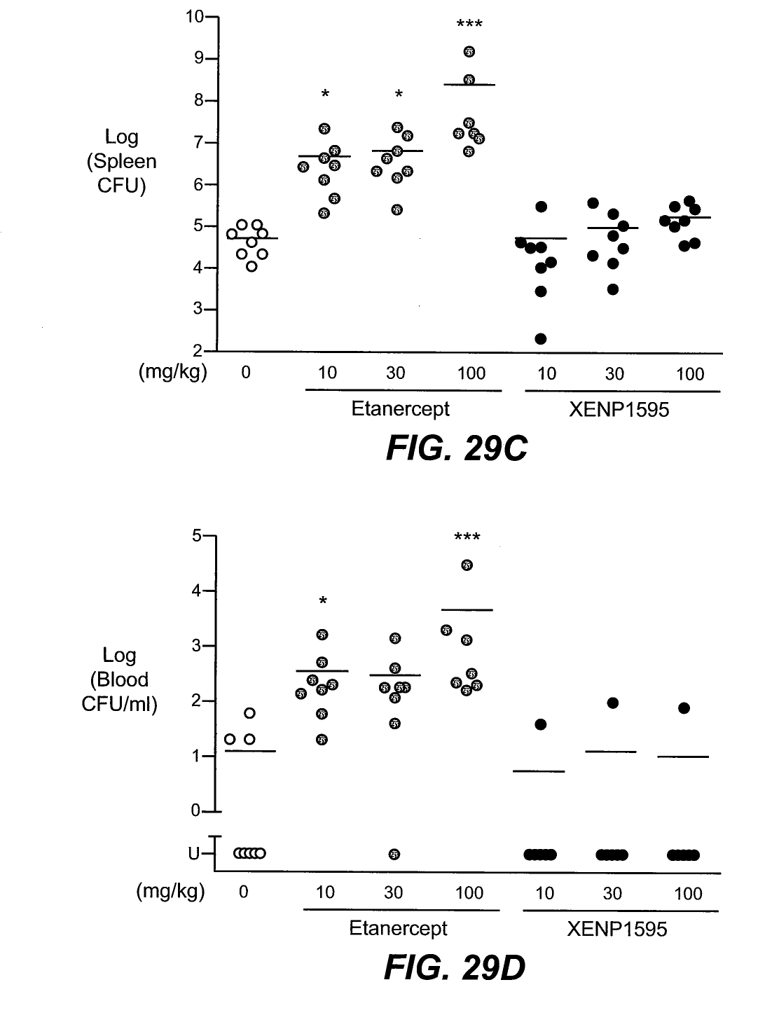

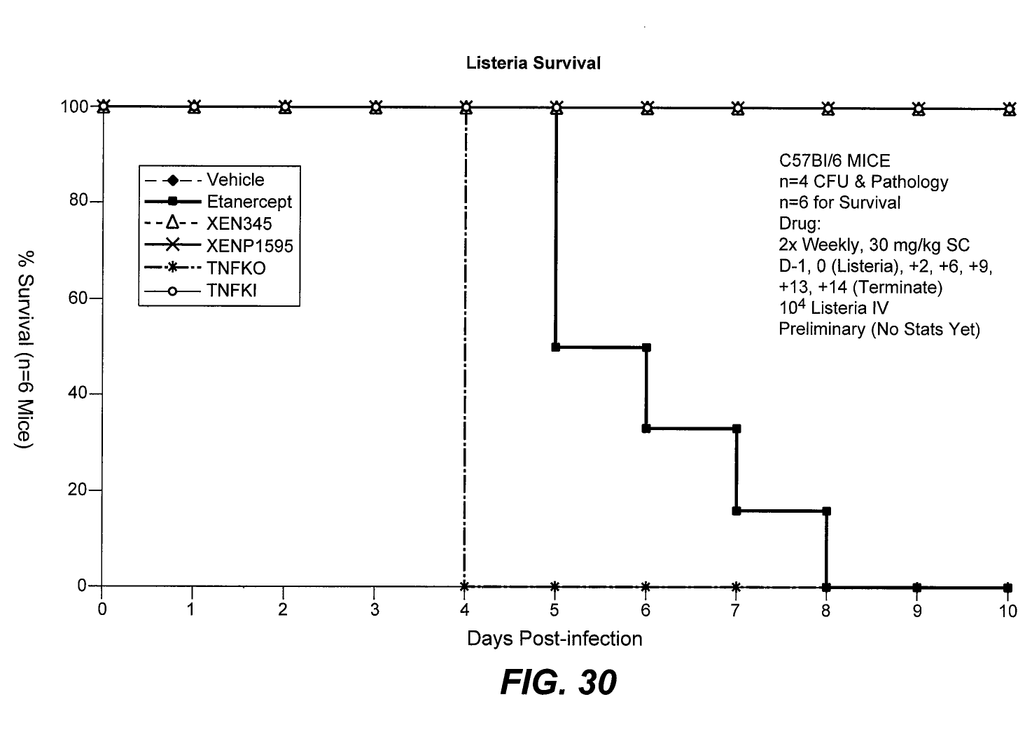

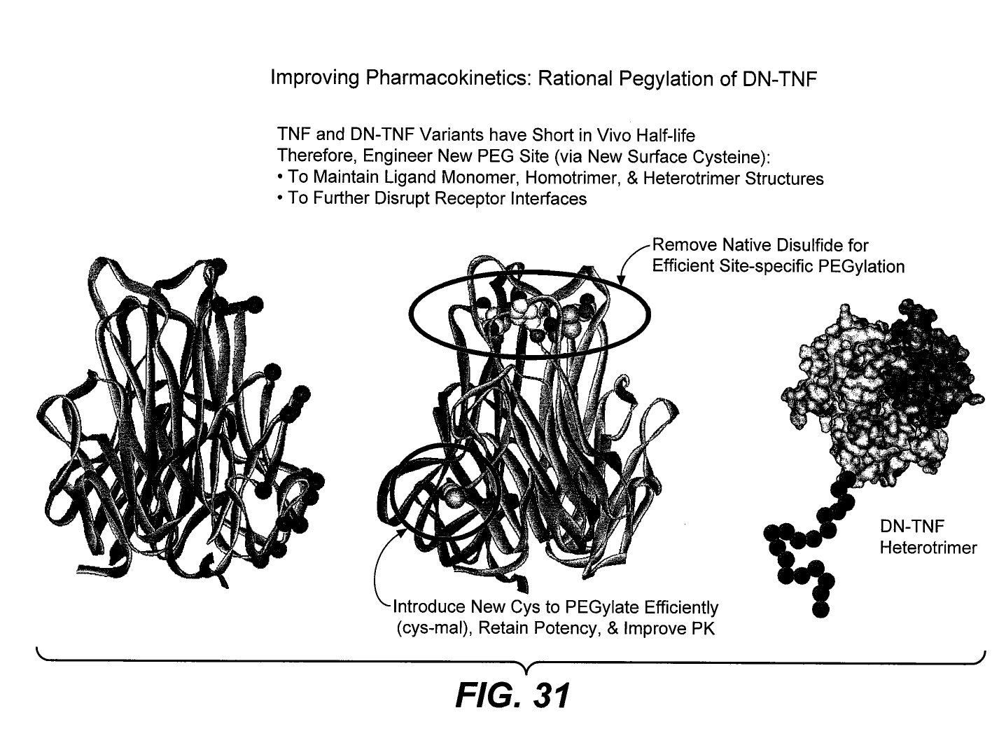

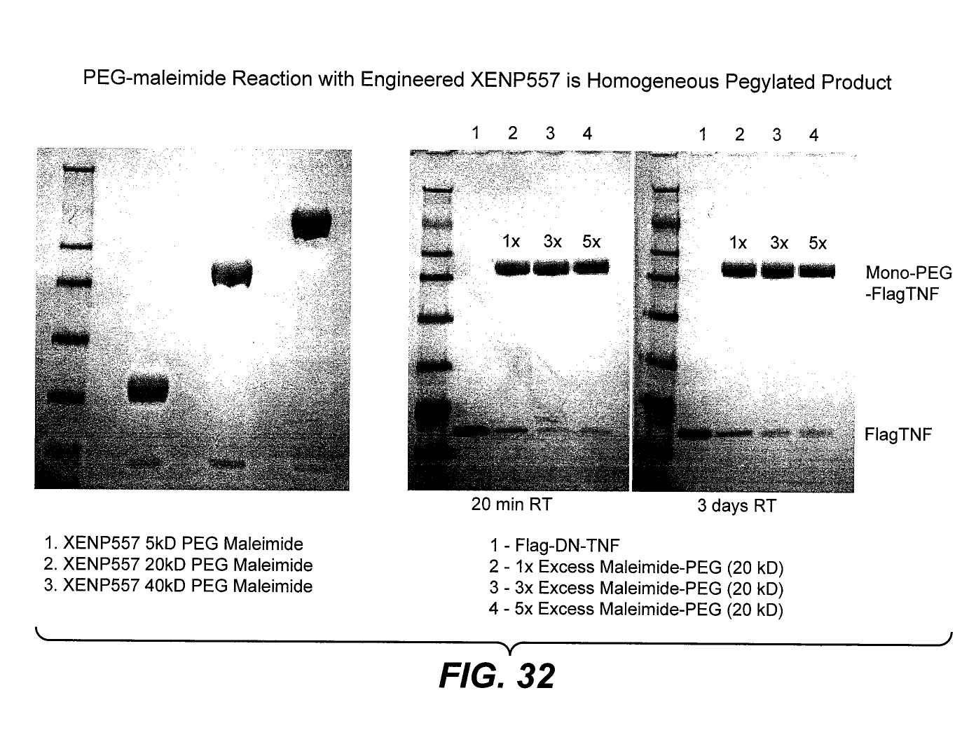

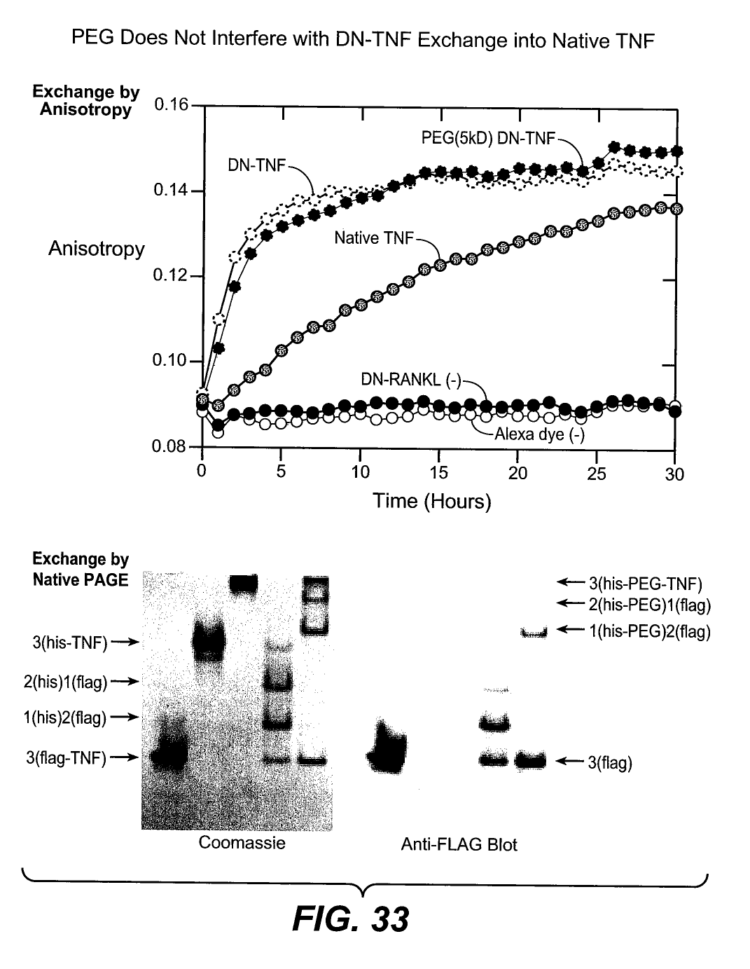

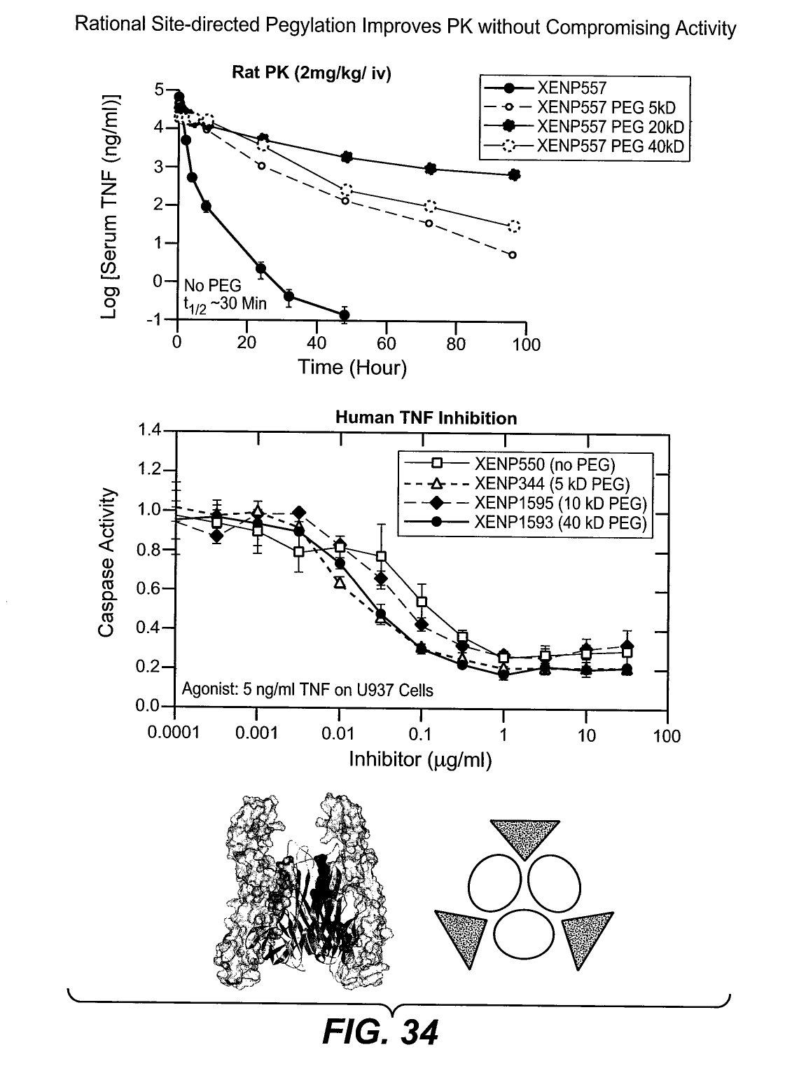

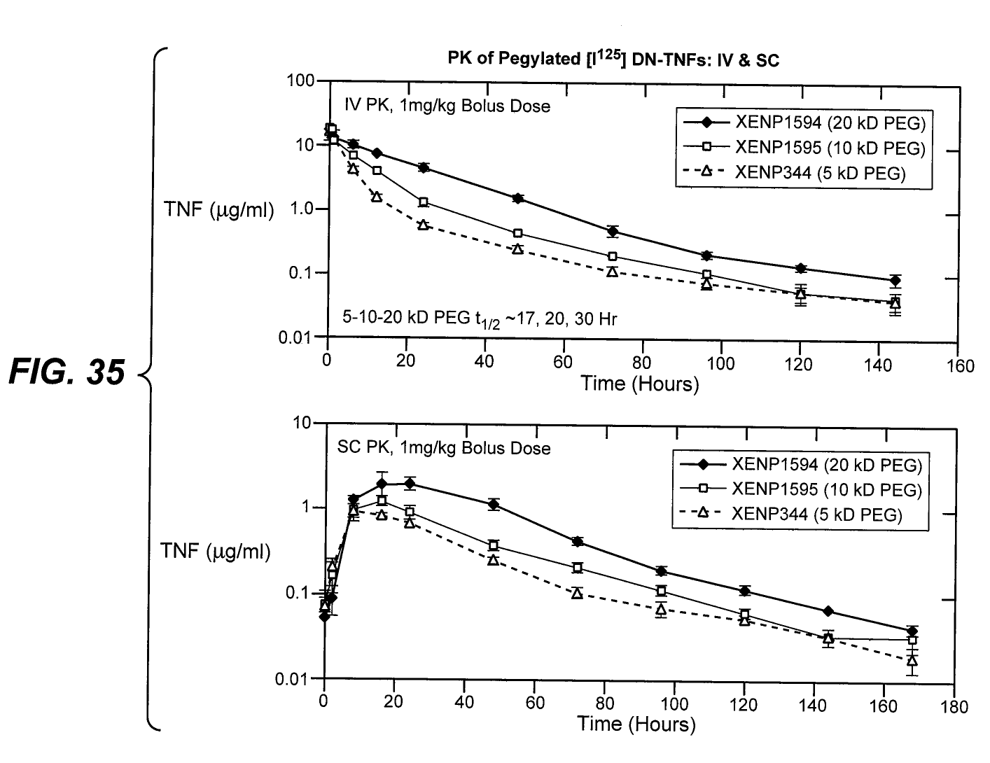

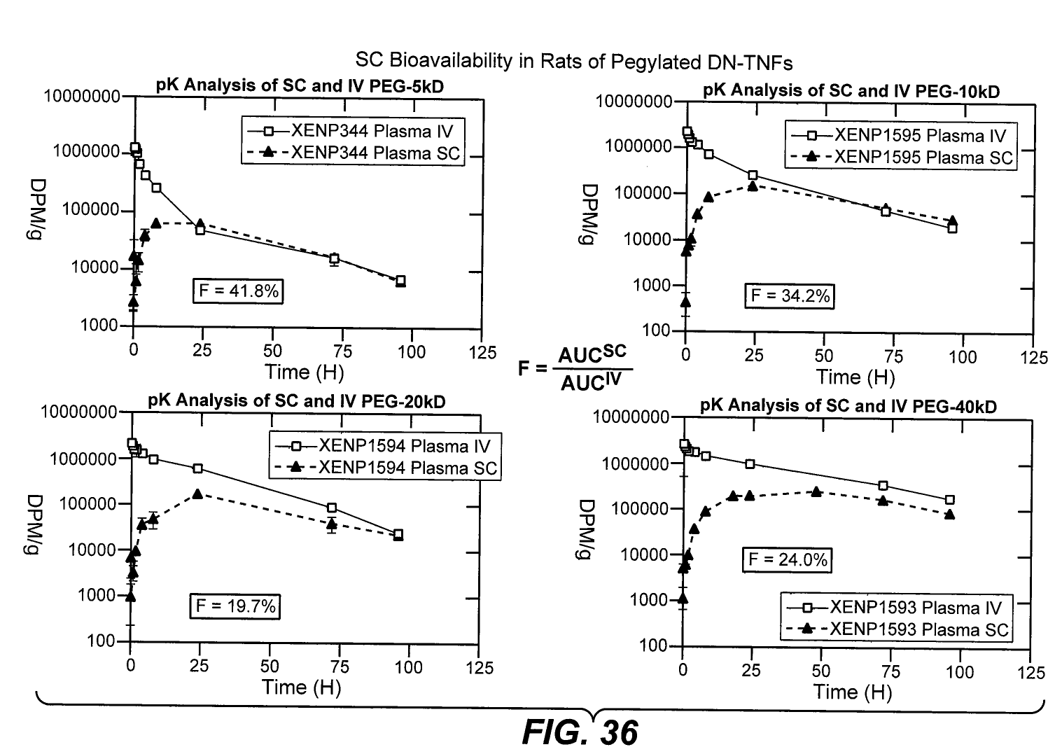

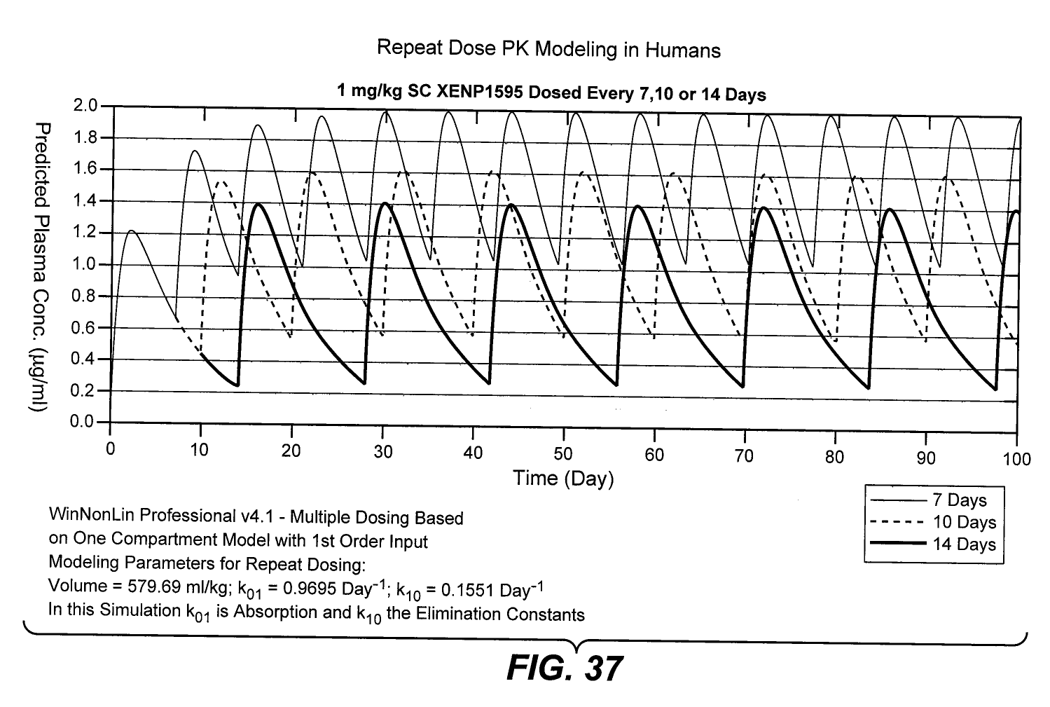

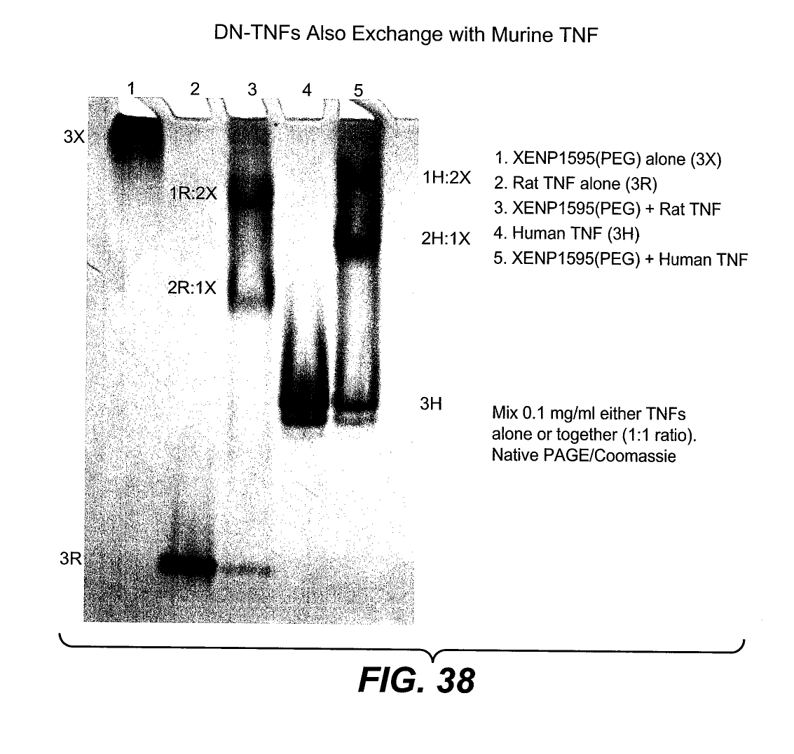

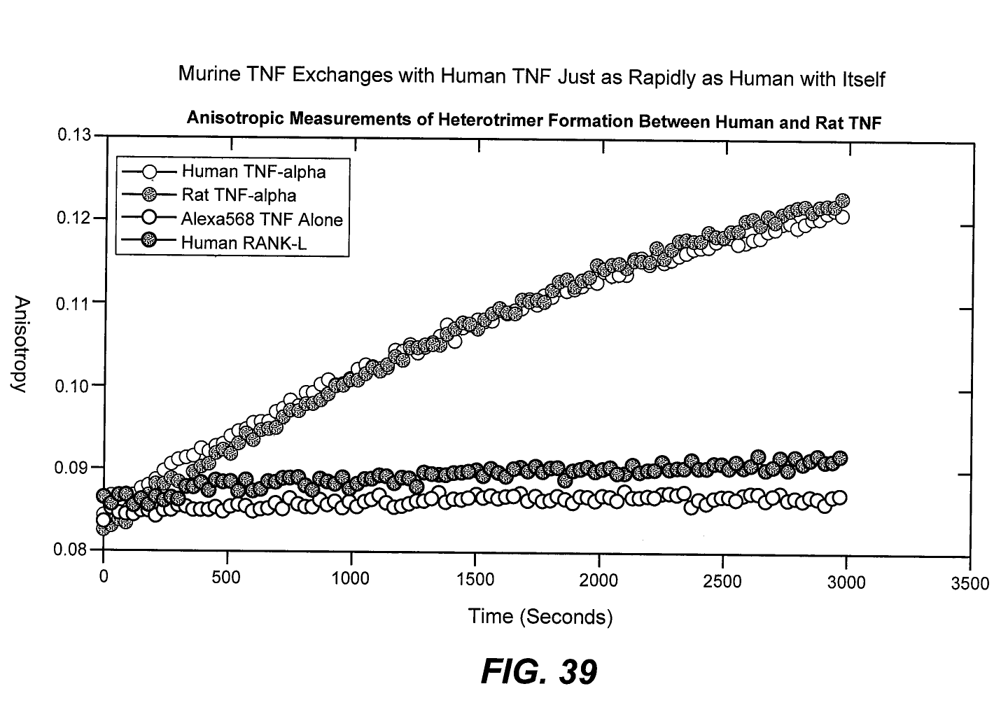

Optimal sites for modification can be chosen using a variety of criteria, including but not limited to, visual inspection, structural analysis, sequence analysis and molecular simulation. For example, as shown in FIG. 18, the fractional accessibility (surface_aa) of individual residues was analyzed to identify mutational sites that will not disrupt the monomer structure. Then the minimum distance (mindistance) from each side chain of a monomer to another subunit was calculated to ensure that chemical modification will not disrupt trimerization. It is possible that receptor binding disruption may occur and may be beneficial to the activity of the TNF variants of this invention. See also FIG. 3139.

In a preferred embodiment, the optimal chemical modification sites for the TNF-α variants of the present invention, include but are not limited to:

| | | <surface> | <min distance> | <combined> |
|---|---|---|---|---|
| GLU | 23 | 0.9 | 0.9 | 0.8 |
| GLN | 21 | 0.8 | 0.9 | 0.7 |
| ASP | 45 | 0.7 | 1.0 | 0.7 |
| ASP | 31 | 0.8 | 0.6 | 0.5 |
| ARG | 44 | 0.6 | 0.9 | 0.5 |
| GLN | 25 | 0.5 | 1.0 | 0.5 |
| GLN | 88 | 0.7 | 0.7 | 0.4 |
| GLY | 24 | 0.5 | 0.9 | 0.4 |
| ASP | 140 | 0.6 | 0.7 | 0.4 |
| GLU | 42 | 0.5 | 0.8 | 0.4 |
| GLU | 110 | 0.8 | 0.4 | 0.4 |
| GLY | 108 | 0.8 | 0.4 | 0.3 |
| GLN | 27 | 0.4 | 0.9 | 0.3 |
| GLU | 107 | 0.7 | 0.4 | 0.3 |
| ASP | 10 | 0.7 | 0.4 | 0.3 |
| SER | 86 | 0.6 | 0.5 | 0.3 |
| ALA | 145 | 0.8 | 0.4 | 0.3 |
| LYS | 128 | 0.6 | 0.4 | 0.3 |
| ASN | 46 | 0.3 | 0.9 | 0.3 |
| LYS | 90 | 0.5 | 0.5 | 0.3 |
| TYR | 87 | 0.6 | 0.4 | 0.3 |

In a more preferred embodiment, the optimal chemical modification sites are 21, 23, 31 and 45, taken alone or in any combination. In an even more preferred embodiment, a TNF-α variant of the present invention include the R31 C mutation. For example, TNF-α variant A145R/197T was evaluated with and without a PEG-10 moiety (which was coupled to R31C).

Optionally, various excipients may be used to catalyze TNF exchange and heterotrimer formation. Other modifications, such as covalent additions, may promote or inhibit exchange, thereby affecting the specificity of the mechanism. The TNF hetero-trimer of the present invention becomes more labile when incubated in the presence of various detergents, lipids or the small molecule suramin. Thus, use of these excipients may greatly enhance the rate of heterotrimer formation. Covalent addition of molecules acting in a similar way may also promote exchange with transmembrane ligand.

Suitable excipients include pharmaceutically acceptable detergents or surfactants (ionic, nonionic, cationic and anionic), lipids, mixed lipid vesicles, or small molecules, including long chain hydrocarbons (straight or branched, substituted or non-substituted, cis-trans saturated or unsaturated) that promote TNF exchange. For example, excipients that are useful in the present invention include (but are not limited to): CHAPS, Deoxycholate, Tween-20, Tween-80, Igepal, SDS, Triton X-100, and Triton X-114, steroidal or bile salts containing detergents (CHAPS), nonionic alkyl ethoxylate derived detergents (e.g., Triton and Tween), ionic detergents (SDS), and steroidal detergents (Deoxycholate). For example, TNF variant A145R/197T blocks transmembrane TNF-induced signaling activity. The steroidal or bile salt containing detergents are preferably used at concentrations above CMC. However, detergents with hydrocarbon tails retain catalytic activity over a much broader concentration range. Certain detergents, especially non-ionic detergents may be used to promote exchange at or below their CMC. The excipients described above are equally useful as excipients in a pharmaceutical formulation of the TNF-α variants of the present invention.

In another preferred embodiment, portions of either the N- or C-termini of the wild type TNF-α monomer are deleted while still allowing the TNF-α molecule to fold properly. In addition, these modified TNF-α proteins would lack receptor binding ability, and could optionally interact with other wild type TNF alpha molecules or modified TNF-α proteins to form trimers as described above. More specifically, removal or deletion of from about 1 to about 55 amino acids from either the N or C termini, or both, are preferred. A more preferred embodiment includes deletions of N-termini beyond residue 10 and more preferably, deletion of the first 47 N-terminal amino acids. The deletion of C-terminal leucine is an alternative embodiment. In another preferred embodiment, the wild type TNF-α or variants generated by the invention may be circularly permuted. All natural proteins have an amino acid sequence beginning with an N-terminus and ending with a C-terminus. The N- and C-termini may be joined to create a cyclized or circularly permutated TNF-α proteins while retaining or improving biological properties (e.g., such as enhanced stability and activity) as compared to the wild-type protein. In the case of a TNF-α protein, a novel set of N- and C-termini are created at amino acid positions normally internal to the protein's primary structure, and the original N- and C-termini are joined via a peptide linker consisting of from 0 to 30 amino acids in length (in some cases, some of the amino acids located near the original termini are removed to accommodate the linker design). In a preferred embodiment, the novel N- and C-termini are located in a non-regular secondary structural element, such as a loop or turn, such that the stability and activity of the novel protein are similar to those of the original protein. The circularly permuted TNF-α protein may be further PEGylated or glycosylated. In a further preferred embodiment PDA® technology may be used to further optimize the TNF-α variant, particularly in the regions created by circular permutation. These include the novel N- and C-termini, as well as the original termini and linker peptide.

Various techniques may be used to permutate proteins. See U.S. Pat. No. 5,981,200; Maki K, Iwakura M., Seikagaku. 2001 January; 73(1): 42-6; Pan T., Methods Enzymol. 2000; 317:313-30; Heinemann U, Hahn M., Prog Biophys Mol Biol. 1995; 64(2-3): 121-43; Harris M E, Pace NR, Mol Biol Rep. 1995-96; 22(23):115-23; Pan T, Uhlenbeck O C., 1993 Mar. 30; 125(2): 111-4; Nardulli AM, Shapiro D J. 1993 Winter; 3(4):247-55, EP 1098257 A2; WO 02/22149; WO 01/51629; WO 99/51632; Hennecke, et al., 1999, J. Mol. Biol., 286, 1197-1215; Goldenberg et al J. Mol. Biol. 165, 407-413 (1983); Luger et al., Science, 243, 206-210 (1989); and Zhang et al., Protein Sci 5, 1290-1300 (1996); all hereby incorporated by reference. In addition, a completely cyclic TNF-α may be generated, wherein the protein contains no termini. This is accomplished utilizing intein technology. Thus, peptides can be cyclized and in particular inteins may be utilized to accomplish the cyclization.

Variant TNF-α polypeptides of the present invention may also be modified in a way to form chimeric molecules comprising a variant TNF-α polypeptide fused to another, heterologous polypeptide or amino acid sequence. In one embodiment, such a chimeric molecule comprises a fusion of a variant TNF-α polypeptide with a tag polypeptide which provides an epitope to which an anti-tag antibody can selectively bind. The epitope tag is generally placed at the amino- or carboxyl-terminus of the variant TNF-α polypeptide. The presence of such epitope-tagged forms of a variant TNF-α polypeptide can be detected using an antibody against the tag polypeptide. Also, provision of the epitope tag enables the variant TNF-α polypeptide to be readily purified by affinity purification using an anti-tag antibody or another type of affinity matrix that binds to the epitope tag. In an alternative embodiment, the chimeric molecule may comprise a fusion of a variant TNF-α polypeptide with an immunoglobulin or a particular region of an immunoglobulin. For a bivalent form of the chimeric molecule, such a fusion could be to the Fc region of an IgG molecule.

Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol. 8:2159-2165 (1988)]; the c-myc tag and the 8F9, 3C7, 6E10, G4, B7 and 9E10 antibodies thereto [Evan et al., Molecular and Cellular Biology, 5:36103616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology 6:1204-1210 (1988)]; the KT3 epitope peptide [Martin et al., Science 255:192-194 (1992)]; tubulin epitope peptide [Skinner et al., J. Biol. Chem. 266:15163-15166 (1991)]; and the T7 gene 10 protein peptide tag [Lutz-Freyermuth et al., Proc. Natl. Acad. Sci. U.S.A. 87:6393-6397 (1990)], all incorporated by reference.

In a preferred embodiment, the variant TNF-α protein is purified or isolated after expression. Variant TNF-α proteins may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, and chromatofocusing. For example, the variant TNF-α protein may be purified using a standard anti-library antibody column. Ultrafiltration and diafiltration techniques, in conjunction with protein concentration, are also useful. For general guidance in suitable purification techniques, see Scopes, R., Protein Purification, Springer-Verlag, NY (1982), incorporated by reference. The degree of purification necessary will vary depending on the use of the variant TNF-α protein. In some instances no purification will be necessary.

The class of Dominant-Negative (DN) TNF compounds is just one example of molecules that can be envisioned to selectively inhibit soluble TNF while sparing the activity of transmembrane TNF. In addition, other classes of inhibitor can be created and/or identified by screening. For example, a soluble TNF-selective antibody can be created a number of ways. Structural prediction tools can be used to identify antibody-binding regions unique to soluble TNF that are masked or sterically blocked in transmembrane TNF. Mice or other animals could then be immunized with peptides or protein fragments or fusion proteins from these TNF domain(s) that are closest to the cell membrane when TNF is in its transmembrane form. Antibodies raised specifically against these regions, because of steric hindrance, would be unlikely to bind to and inactivate transmembrane TNF. As an alternate approach, the common surface-exposed surfaces of TNF distal to the cell membrane could be blocked (chemically, such as by pegylation, or with binding or fusion proteins) before immunization. Antibodies raised with these antigens would thus be more likely to bind to the TNF surface closest to the cell membrane. These approaches could be combined through mixed immunization and boost. For example, antibodies raised to normal native soluble TNF in the primary immunization could be boosted with peptide or protein fragments from soluble TNF that are not exposed in membrane-bound TNF. As another example, peptides or small molecules can be identified that bind only to soluble TNF. As above, structural prediction tools can be used to identify surface regions unique to soluble TNF. Small molecules or peptides binding to these regions could be identified through modeling approaches, or by screening for compounds that bind specifically to soluble TNF but not transmembrane TNF. Even without specific immunization approaches, inhibitors could be screened for soluble vs. transmembrane selectivity using two assays, one specific for soluble TNF activity (e.g., caspase activation by recombinant soluble human TNF), and one specific for transmembrane TNF activity (e.g., caspase activation by membrane-fused transmembrane TNF lacking the TNF Convertase (TACE) protease cleavage site, or blocked from release by a TACE inhibitor). Finally, even without specifically screening for soluble TNF selectivity in binding assays or cell assays, antibodies or small molecules could be screened in animal models of infection vs. efficacy to determine if a given compound had the desired safety (e.g., lack of suppression of host resistance to infection due to sparing of transmembrane TNF activity) vs. efficacy (e.g., anti-inflammatory effect in arthritis or other disease models due to inhibition of soluble TNF activity).

In addition, the invention provides methods of screening candidate agents for selective inhibitors (e.g. inhibition of soluble TNF-α activity while substantially maintaining transmembrane TNF-α activity). In general, this is done in a variety of ways as is known in the art, and can include a first assay to determine whether the candidate agent binds to soluble TNF-α and transmembrane TNF-α, and then determining the effect on biological activity. Alternatively, just activity assays can be done. In general, a candidate agent (usually a library of candidate agents) are contacted with a soluble TNFa protein and activity is assayed, and similarly with the transmembrane TNF-α protein (usually as part of a cell).

A wide variety of suitable assay formats will be apparent by those in the art. In a preferred embodiment of the methods herein, one member of the assay, e.g. the candidate agent and the wild-type TNF-α (either soluble or transmembrane), is non-diffusably bound to an insoluble support having isolated sample receiving areas (e.g. a microtiter plate, an array, etc.; alternatively bead formats such as are used in high throughput screening using FACS can be used). The insoluble support may be made of any composition to which the protein or the candidate agent can be bound, is readily separated from soluble material, and is otherwise compatible with the overall method of screening. The surface of such supports may be solid or porous and of any convenient shape. Examples of suitable insoluble supports include microtiter plates, arrays, membranes and beads. These are typically made of glass, plastic (e.g., polystyrene), polysaccharides, nylon or nitrocellulose, teflon TM, etc. Microtiter plates and arrays are especially convenient because a large number of assays can be carried out simultaneously, using small amounts of reagents and samples. The particular manner of binding the protein or the candidate agent is not crucial so long as it is compatible with the reagents and overall methods of the invention, maintains the activity of the composition and is nondiffusable. Preferred methods of binding include the use of antibodies (which do not sterically block either the ligand binding site or activation sequence when the protein is bound to the support), direct binding to “sticky’ or ionic supports, chemical crosslinking, the synthesis of the protein or agent on the surface, etc. Following binding of the protein or candidate agent, excess unbound material is removed by washing. The sample receiving areas may then be blocked through incubation with bovine serum albumin (BSA), casein or other innocuous protein or other moiety.

In a preferred embodiment, the protein is bound to the support, and a candidate bioactive agent is added to the assay. Alternatively, the candidate agent is bound to the support and the protein is added.

In some embodiments, one of the members of the assay (usually the nonbound component) can be labeled (e.g. optical dyes such as fluorophores and chromophores, enzymes, magnetic particles, radioisotopes, etc.), to detect binding after washing unbound reagent. Activity assays are described herein, including but not limited to, caspase assays, TNF-α cytotoxicity assays, DNA binding assays; transcription assays (using reporter constructs; see Stavridi, supra); size exclusion chromatography assays and radiolabeling/immuno-precipitation; see Corcoran et al., supra); and stability assays (including the use of circular dichroism (CD) assays and equilibrium studies; see Mateu, supra); all of which are incorporated by reference.

“Candidate agent” or “candidate drug” as used herein describes any molecule, e.g., proteins including biotherapeutics including antibodies and enzymes, small organic molecules including known drugs and drug candidates, polysaccharides, fatty acids, vaccines, nucleic acids, etc. that can be screened for activity as outlined herein. Candidate agents are evaluated in the present invention for discovering potential therapeutic agents that affect RR activity and therefore potential disease states, for elucidating toxic effects of agents (e.g. environmental pollutants including industrial chemicals, pesticides, herbicides, etc.), drugs and drug candidates, food additives, cosmetics, etc., as well as for elucidating new pathways associated with agents (e.g. research into the side effects of drugs, etc.).

Candidate agents encompass numerous chemical classes. In one embodiment, the candidate agent is an organic molecule, preferably small organic compounds having a molecular weight of more than 100 and less than about 2,500 daltons. Particularly preferred are small organic compounds having a molecular weight of more than 100 and less than about 2,000 daltons, more preferably less than about 1500 daltons, more preferably less than about 1000 daltons, more preferably less than 500 daltons. Candidate agents comprise functional groups necessary for structural interaction with proteins, particularly hydrogen bonding, and typically include at least one of an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

“Known drugs” or “known drug agents” or “already-approved drugs” refers to agents (i.e., chemical entities or biological factors) that have been approved for therapeutic use as drugs in human beings or animals in the United States or other jurisdictions. In the context of the present invention, the term “already-approved drug” means a drug having approval for an indication distinct from an indication being tested for by use of the methods disclosed herein. Using psoriasis and fluoxetine as an example, the methods of the present invention allow one to test fluoxetine, a drug approved by the FDA (and other jurisdictions) for the treatment of depression, for effects on biomarkers of psoriasis (e.g., keratinocyte proliferation or keratin synthesis); treating psoriasis with fluoxetine is an indication not approved by FDA or other jurisdictions. In this manner, one can find new uses (in this example, anti-psoriatic effects) for an already-approved drug (in this example, fluoxetine).

Candidate agents are obtained from a wide variety of sources including libraries of synthetic or natural compounds. For example, numerous means are available for random and directed synthesis of a wide variety of organic compounds and biomolecules, including expression and/or synthesis of randomized oligonucleotides and peptides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

In one embodiment, the candidate bioactive agents are proteins as described herein. In a preferred embodiment, the candidate bioactive agents are naturally occuring proteins or fragments of naturally occuring proteins. Thus, for example, cellular extracts containing proteins, or random or directed digests of proteinaceous cellular extracts, may be used. In this way libraries of procaryotic and eucaryotic proteins may be made for screening in the systems described herein. Particularly preferred in this embodiment are libraries of bacterial, fungal, viral, and mammalian proteins, with the latter being preferred, and human proteins being especially preferred.

In a preferred embodiment, the candidate agents are antibodies, a class of proteins. The term “antibody” includes full-length as well antibody fragments, as are known in the art, including Fab Fab2, single chain antibodies (Fv for example), chimeric antibodies, humanized and human antibodies, etc., either produced by the modification of whole antibodies or those synthesized de novo using recombinant DNA technologies, and derivatives thereof.

In a preferred embodiment, the candidate bioactive agents are nucleic acids, particularly those with alternative backbones or bases, comprising, for example, phosphoramide (Beaucage, et al., Tetrahedron, 49(10):1925 (1993) and references therein; Letsinger, J. Org. Chem., 35:3800 (1970); Sprinzl, et al., Eur. J. Biochem., 81:579 (1977); Letsinger, et al., Nucl. Acids Res., 14:3487 (1986); Sawai, et al., Chem. Lett., 805 (1984), Letsinger, et al., J. Am. Chem. Soc., 110:4470 (1988); and Pauwels, et al., Chemica Scripta, 26:141 (1986)), phosphorothioate (Mag, et al., Nucleic Acids Res., 19:1437 (1991); and U.S. Pat. No. 5,644,048), phosphorodithioate (Briu, et al., J. Am. Chem. Soc., 111:2321 (1989)), O-methylphophoroamidite linkages (see Eckstein, Oligonucleotides and Analogues: A Practical Approach, Oxford University Press), and peptide nucleic acid backbones and linkages (see Egholm, J. Am. Chem. Soc., 114:1895 (1992); Meier, et al., Chem. Int. Ed. Engl., 31:1008 (1992); Nielsen, Nature, 365:566 (1993); Carlsson, et al., Nature, 380:207 (1996), all of which are incorporated by reference)). Other analog nucleic acids include those with positive backbones (Denpcy, et al., Proc. Natl. Acad. Sci. USA, 92:6097 (1995)); non-ionic backbones (U.S. Pat. Nos. 5,386,023; 5,637,684; 5,602,240; 5,216,141; and 4,469,863; Kiedrowshi, et al., Angew. Chem. Intl. Ed. English, 30:423 (1991); Letsinger, et al., J. Am. Chem. Soc., 110:4470 (1988); Letsinger, et al., Nucleoside & Nucleotide, 13:1597 (1994); Chapters 2 and 3, ASC Symposium Series 580, “Carbohydrate Modifications in Antisense Research”, Ed. Y. S. Sanghui and P. Dan Cook; Mesmaeker, et al., Bioorganic & Medicinal Chem. Lett., 4:395 (1994); Jeffs, et al., J. Biomolecular NMR, 34:17 (1994); Tetrahedron Lett., 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook, and peptide nucleic acids. Nucleic acids containing one or more carbocyclic sugars are also included within the definition of nucleic acids (see Jenkins, et al., Chem. Soc. Rev., (1995) pp. 169-176). Several nucleic acid analogs are described in Rawls, C & E News, Jun. 2, 1997, page 35. All of these references are hereby expressly incorporated by reference. These modifications of the ribose-phosphate backbone may be done to facilitate the addition of additional moieties such as labels, or to increase the stability and half-life of such molecules in physiological environments. In addition, mixtures of naturally occurring nucleic acids and analogs can be made. Alternatively, mixtures of different nucleic acid analogs, and mixtures of naturally occuring nucleic acids and analogs may be made. The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribonucleotides, and any combination of bases, including uracil, adenine, thymine, cytosine, guanine, inosine, xathanine hypoxathanine, isocytosine, isoguanine, 4-acetylcytosine, 8-hydroxy-N-6- methyladenosine, aziridinylcytosine, pseudoisocytosine, 5-(carboxyhydroxylmethyl)uracil, 5-fluorouracil, 5-bromouracil, 5-carboxymethylaminomethyl -2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5-methoxycarbonylmethyluracil, 5-methoxyuracil, 2-methylthio-N-6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methyl ester, uracil-5-oxyacetic acid, pseudouracil, queosine, 2-thiocytosine, and 2,6-diaminopurine.etc.

As described above generally for proteins, nucleic acid candidate bioactive agents may be naturally occuring nucleic acids, random and/or synthetic nucleic acids. For example, digests of procaryotic or eucaryotic genomes may be used as is outlined above for proteins. In addition, RNA is are included herein.

Once made, the variant TNF-α proteins and nucleic acids of the invention find use in a number of applications. In a preferred embodiment, the variant TNF-α proteins are administered to a patient to treat a TNF-α related disorder. By “TNF-α related disorder” or “TNF-α responsive disorder” or “condition” herein is meant a disorder that may be ameliorated by the administration of a pharmaceutical composition comprising a variant TNF-α protein, including, but not limited to, neurologic, pain, pulmonary, hematological, oncology, inflammatory and immunological disorders. The variant TNF-α is a major effector and regulatory cytokine with a pleiotropic role in the pathogenesis of diseases, including immune-regulated diseases, fibrosis conditions, oncological conditions, and inflammation related conditions. In a preferred embodiment, the variant TNF-α protein is used to treat arthritis, psoriatic arthritis, ankkylosing spondylitis, spondyloarthritis, spondyloarthropathies, rheumatoid arthritis, juvenile rheumatoid arthritis, juvenile idiopathic arthritis, schleroderma, Sjogren's syndrome, TRAPS, periodic fever, periprosthetic osteolysis, apthous stomatitis, pyoderma gangrenosum, uveitis, reticulohistiocystosis, inflammatory bowel diseases, sepsis and septic shock, Crohn's Disease, psoriasis, graft versus host disease (GVHD) and hematologic malignancies, such as multiple myeloma (MM), refractory MM, myelodysplastic syndrome (MDS) idiopathic thrombocytopenic purpura, ovarian carcinoma, and acute myelogenous leukemia (AML), cancer and the inflammation associated with tumors, pain, including spinal disk pain, chronic lower back pain chronic neck pain, pain due to bone metastasis, pain and swelling after molar extraction, neurological conditions and neural damage conditions such as peripheral nerve injury, demyelinating diseases, sciatica, autoimmune sensorineural hearing loss, CIDP, Alzheimers disease, Parkinson's disease, diabetes, insulin resistance, insulin sensitivity, Syndrome X, Wegener's Granulomatosis, dermatomyositis, histicytosis, polymyositis, psoriasis, plaque psoriasis, cancer cachexia, temporomandibular disorders, refractory ocular sarcoidosis, sarcoidosis, behcet's, churg-strauss syndrome, asthma, idiopatic pneumonia following bone marrow transplantation, systemic lupus erythematosus (SLE), lupus nephritis, atherosclerosis, polyneuropathy, orangomegaly, endocrinopathy, M protein, skin changes (POEMS syndrome), Sneddon-Wilkinson disease, necrotizing crescentic glomerulonephritis, renal amyloidosis, AA amyloidosis, erythema nodosum leprosum (ENL), chronic kidney disease, malnutrition, inflammation and atherosclerosis (MIA) syndrome, COPD, pulmonary fibrosis, nephritis, and Waldenstrom's macroglobulinemia. See for example, Tsimberidou et al., Expert Rev Anticancer Ther 2002 June;2(3):277-86, U.S. Pat. No. 6,015,557; Present, D H, et al., N Engl J Med 1999 340: 1398-1405; Braun, Jet al Lnacet 2002; 359: 1187-93; Williams, J D and Griffiths, C E, Clin Exp Dermatol. 2002 October; 27(7) 585-90. Chin, R L et la, J. Neurol. Sci 2003 Jun. 15: 210(1-2) 19-21; Lovell, D J et al., N Engl. J. Med 2000; 342: 763-769; Lorenz, H M and Kalden, JR Arthritis Res. 2002; 4 Suppl 3:S17-24; Kalden, JR, Arthritis Res 2002; 27(7): 585-90; Mease, P J et al., Lancet. 2000 Jul. 29:356(9227): 385-90; Gorman, J D, et al., N Engl J Med 2000; 346: 1349-1356; Anderson, V C and Israel, Z, Curr Rev Pain 2000; 4(2): 105-111; Marshall, L L and Trethewie, ER, Lancet 291973) 320; Takahashi, H, et al., Spine, 21 (1996) 218-224; Igarashi, T, et al., Spine, 25 (2000) 2975-2980; Wagner, R and Myers, RR Neuroreport., 7 (1996) 2897-2901; Olmarker, K and Rydevik, B, Spine, 26 (2001) 863-869; Sommer, C et al., J Peripher Nery Syst, 6 (2001) 67-72; Tobinick E, Curr Med Res Opin, July 2004; 20(7) 1075-1085; Genevay, S et al., Ann Rheum Dis 2004: doi:10.1136/ard.2003.016451; Tobinick, E, Clinical Therapeutics August 2003. 25(8): 2279-88; Wu, S, et al. Cancer Res. 1993 Apr. 15; 538): 1939-44; all Baughman, R P et al., Chest. 2005 August; 128(2): 1062-47; Gherardi, r K et al., Ann Neurol 1994 April; 35(4): 501-5; Van den Bosch, F., et al., Ann Rheum Dis. 2001 Nov: 60 Suppl 3: iii33-6; Voigtlander, C, et al., Arch Dermatol. 2001 December; 137(12): 1571-4 Zaenker, M et al., Int J Tissue React. 2004; 26(3-4): 85-92; Smith, G R et al., Intern Med J. 2004 Sep-Oct; 34(9-10): 570-2; Tsimberidou, A M et al., Leuk Res 2003 May; 27(5): 375-80; Macdougall, IC Nephrol Dial Transplant. 2004 August; 19 Suppl 5:V73-78; all entirely incorporated by reference.

The TNF-α variants of the present invention are preferably used to treat RA, juvenile RA, psoriatic arthritis, ankylosing spondylitis, psoriasis, Crohn's, IBD, and ulcerative colitis.

Inflammatory bowel disease (“IBD”) is the term generally applied to two diseases, namely ulcerative colitis and Crohn's disease. Ulcerative colitis is a chronic inflammatory disease of unknown etiology afflicting only the large bowel and, except when very severe, limited to the bowel mucosa. The course of the disease may be continuous or relapsing, mild or severe. It is curable by total colostomy which may be needed for acute severe disease or chronic unremitting disease. Crohn's disease is also a chronic inflammatory disease of unknown etiology but, unlike ulcerative colitis, it can affect any part of the bowel. Although lesions may start superficially, the inflammatory process extends through the bowel wall to the draining lymph nodes. As with ulcerative colitis, the course of the disease may be continuous or relapsing, mild or severe but, unlike ulcerative colitis, it is not curable by resection of the involved segment of bowel. Most patients with Crohn's disease come to surgery at some time, but subsequent relapse is common and continuous medical treatment is usual. Remicade® (inflixmab) is the commercially available treatment for Crohn's disease. Remicade® is a chimeric monoclonal antibody that binds to TNF-α. The use of the TNF-α variants of the present invention may also be used to treat the conditions associated with IBD or Crohn's Disease.

“Sepsis” is herein defined to mean a disease resulting from gram positive or gram negative bacterial infection, the latter primarily due to the bacterial endotoxin, lipopolysaccharide (LPS). It can be induced by at least the six major gram-negative bacilli and these are Pseudomonas aeruginosa, Escherichia coli, Proteus, Klebsiella, Enterobacter and Serratia. Septic shock is a condition which may be associated with Gram positive infections, such as those due to pneumococci and streptococci, or with Gram negative infections, such as those due to Escherichia coli, Klebsiella-Enterobacter, Pseudomonas, and Serratia. In the case of the Gram-negative organisms the shock syndrome is not due to bloodstream invasion with bacteria per se but is related to release of endotoxin, the LPS moiety of the organisms' cell walls, into the circulation. Septic shock is characterized by inadequate tissue perfusion and circulatory insufficiency, leading to insufficient oxygen supply to tissues, hypotension, tachycardia, tachypnea, fever and oliguria. Septic shock occurs because bacterial products, principally LPS, react with cell membranes and components of the coagulation, complement, fibrinolytic, bradykinin and immune systems to activate coagulation, injure cells and alter blood flow, especially in the microvasculature. Microorganisms frequently activate the classic complement pathway, and endotoxin activates the alternate pathway.

The TNF-α variants of the present invention effectively antagonize the effects of wild type TNF-α-induced cytotoxicity and interfere with the conversion of TNF into a mature TNF molecule (e.g. the trimer form of TNF). Thus, administration of the TNF variants can ameliorate or eliminate the effects of sepsis or septic shock, as well as inhibit the pathways associated with sepsis or septic shock. Administration may be therapeutic or prophylactic. The TNF-α variants of the present invention effectively antagonize the effects of wild type TNF-α-induced cytotoxicity in cell based assays and animal models of peripheral nerve injury and axonal demyelination/degeneration to reduce the inflammatory component of the injury or demyelinating insult. This is believed to critically contribute to the neuropathological and behavioral sequelae and influence the pathogenesis of painful neuropathies.

Severe nerve injury induces activation of Matrix Metallo Proteinases (MMPs), including TACE, the TNF-α-converting enzyme, resulting in elevated levels of TNF-α protein at an early time point in the cascade of events that leads up to Wallerian nerve degeneration and increased pain sensation (hyperalgesia). The TNF-α variants of the present invention antagonize the activity of these elevated levels of TNF-α at the site of peripheral nerve injury with the intent of reducing macrophage recruitment from the periphery without negatively affecting remyelination. Thus, reduction of local TNF-induced inflammation with these TNF-α variants would represent a therapeutic strategy in the treatment of the inflammatory demyelination and axonal degeneration in peripheral nerve injury as well as the chronic hyperalgesia characteristic of neuropathic pain states that often results from such peripheral nerve injuries.

Intraneural administration of exogenous TNF-α produces inflammatory vascular changes within the lining of peripheral nerves (endoneurium) together with demyelination and axonal degeneration (Redford et al 1995). After nerve transection, TNF-positive macrophages can be found within degenerating fibers and are believed to be involved in myelin degradation after axotomy (Stoll et al 1993). Furthermore, peripheral nerve glia (Schwann cells) and endothelial cells produce extraordinary amounts of TNF-α at the site of nerve injury (Wagner et al 1996) and intraperitoneal application of anti-TNF antibody significantly reduces the degree of inflammatory demyelination strongly implicating a pathogenic role for TNF-α in nerve demyelination and degeneration (Stoll et al., 1993). Thus, administration of an effective amount of the TNF-α variants of the present invention may be used to treat these peripheral nerve injury or demyelinating conditions, as well as Alzheimers disease and Parkinson's disease. In a preferred embodiment, a therapeutically effective dose of a variant TNF-α protein is administered to a patient in need of treatment. By “therapeutically effective dose” herein is meant a dose that produces the effects for which it is administered. The exact dose will depend on the purpose of the treatment, and will be ascertainable by one skilled in the art using known techniques. In a preferred embodiment, dosages of about 5 μg/kg are used, administered either intravenously or subcutaneously. As is known in the art, adjustments for variant TNF-α protein degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

A “patient” for the purposes of the present invention includes both humans and other animals, particularly mammals, and organisms. Thus the methods are applicable to both human therapy and veterinary applications. In the preferred embodiment the patient is a mammal, and in the most preferred embodiment the patient is human. The term “treatment” in the instant invention is meant to include therapeutic treatment, as well as prophylactic, or suppressive measures for the disease or disorder. Thus, for example, successful administration of a variant TNF-α protein prior to onset of the disease results in “treatment” of the disease. As another example, successful administration of a variant TNF-α protein after clinical manifestation of the disease to combat the symptoms of the disease comprises “treatment” of the disease. “Treatment” also encompasses administration of a variant TNF-α protein after the appearance of the disease in order to eradicate the disease. Successful administration of an agent after onset and after clinical symptoms have developed, with possible abatement of clinical symptoms and perhaps amelioration of the disease, comprises “treatment” of the disease. Those “in need of treatment” include mammals already having the disease or disorder, as well as those prone to having the disease or disorder, including those in which the disease or disorder is to be prevented.

In another embodiment, a therapeutically effective dose of a variant TNF-α protein, a variant TNFa gene, or a variant TNF-α antibody is administered to a patient having a disease involving inappropriate expression of TNF-α. A “disease involving inappropriate expression of at TNF-α” within the scope of the present invention is meant to include diseases or disorders characterized by aberrant TNF-α, either by alterations in the amount of TNF-α present or due to the presence of mutant TNF-α. An overabundance may be due to any cause, including, but not limited to, overexpression at the molecular level, prolonged or accumulated appearance at the site of action, or increased activity of TNF-α relative to normal. Included within this definition are diseases or disorders characterized by a reduction of TNF-α. This reduction may be due to any cause, including, but not limited to, reduced expression at the molecular level, shortened or reduced appearance at the site of action, mutant forms of TNF-α, or decreased activity of TNF-α relative to normal. Such an overabundance or reduction of TNF-α can be measured relative to normal expression, appearance, or activity of TNF-α according to, but not limited to, the assays described and referenced herein.

The administration of the variant TNF-α proteins of the present invention, preferably in the form of a sterile aqueous solution, may be done in a variety of ways, including, but not limited to, orally, subcutaneously, intravenously, intranasally, transdermally, intraperitoneally, intramuscularly, intrapulmonary, vaginally, rectally, or intraocularly. In some instances, for example, in the treatment of wounds, inflammation, etc., the variant TNF-α protein may be directly applied as a solution, salve, cream or spray. The TNF-α molecules of the present may also be delivered by bacterial or fungal expression into the human system (e.g., WO 04046346 A2, hereby incorporated by reference). Depending upon the manner of introduction, the pharmaceutical composition may be formulated in a variety of ways. The concentration of the therapeutically active variant TNF-α protein in the formulation may vary from about 0.1 to 100 weight %. In another preferred embodiment, the concentration of the variant TNF-α protein is in the range of 0.003 to 1.0 molar, with dosages from 0.03, 0.05, 0.1, 0.2, and 0.3 millimoles per kilogram of body weight being preferred.

The pharmaceutical compositions of the present invention comprise a variant TNF-α protein in a form suitable for administration to a patient. In the preferred embodiment, the pharmaceutical compositions are in a water soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers such as NaOAc; fillers such as microcrystalline cellulose, lactose, corn and other starches; binding agents; sweeteners and other flavoring agents; coloring agents; and polyethylene glycol. Additives are well known in the art, and are used in a variety of formulations. In a further embodiment, the variant TNF-α proteins are added in a micellular formulation; see U.S. Pat. No. 5,833,948, hereby incorporated by reference. Alternatively, liposomes may be employed with the TNF-α proteins to effectively deliver the protein. Combinations of pharmaceutical compositions may be administered. Moreover, the TNF-α compositions of the present invention may be administered in combination with other therapeutics, either substantially simultaneously or co-administered, or serially, as the need may be.

The present invention relates to the discovery that TNF-α variants of the present invention may be administered to patients suffering from a TNF-related condition as adjunctive and/or concomitant therapy to another therapeutic agent, with good to excellent alleviation of the signs and symptoms of the disease, and significant improvement in duration of clinical response. In one preferred embodiment, the treatment and/or prevention of a TNF-related condition involves co-administering another therapeutic agent and a TNF-α variant of the present invention to the individual in therapeutically effective amounts. The TNF-α variant of the present invention and other therapeutic modalilty may be administered substantially simultaneously or sequentially. The TNF-α variant of the present invention and the other therapeutic agent may each be administered in single or multiple doses. Other therapeutic regimens and agents can be used in combination with the therapeutic co-administration of TNF-α variant of the present invention and methotrexate or other drugs that suppress the immune system.

In the case of a patient with rheumatoid arthritis, the benefits of combination therapy with methotrexate and TNF antagonists include high clinical response rates for significantly longer durations in comparison with that obtained with treatment with each therapeutic agent separately.

Other Therapeutic agents for rheumatoid arthritis, include but are not limited to Non-steroidal anti-inflammation drugs (NSAIDs), Disease-modifying antip rheumatic drugs (DMARDs), Steroids and other TNF-α modulating drugs.

Suitable NSAIDs include but are not limited to aspirin, ibuprofen, diclofenac (Voltaren®), selective COX-2 inhibitors (e.g., Vioxx® (rofecoxib, Merck), Bextra® (valdecoxib, Pfizer), and Celebrex® (celecoxib, Pfizer)), etodolac (Lodine®, Wyeth), fenoprofen (Nalfon®), flurbiprofen, indomethacin (Indocin®), ketoprofen, meclofenamate (Ponstel®), meloxicam (Mobic® Boehringer Ingleheim), nabumetone (Relafen®, GlaxoSmithKline), naproxen (Naprosyn®), oxaprozin (Daypro®), choline salicylate (Trilsate®), piroxicam (Feldene®), salsalate, sulindac (Clinoril®), tolmetin, and combinations thereof.

Suitable DMARDs include but are not limited to methotrexate (MTX), sulphasalazine (SSZ), hydroxychloroquine (HCQ), D-penicillamine (DP), aazathioprine (AZA), lefluonimide (LEF, Arava®, Aventis), etanercept (Enbrel®, Immunex/Amgen), infliximab (Remicade® J&J), anakinra (Kineret®, Amgen), azathioprine, D-penicillamine, intramuscular or oral gold (e.g., aurothioglucose, aurothiomalate, auranofin), minocycline, cyclosporine, glucocorticoids, staphylococcal protein A immunoadsorption, and combinations thereof.

The most preferred DMARD is Methotrexate. Methotrexate is commercially available in oral and intravenous formulations, including but not limited to Heumatrex® methotrexate dose pack (Lederle Laboratories); methotrexate tablets from Mylan Pharmaceuticals Inc. and Roxane Laboratories, Inc.; and methotrexate sodium tablets, for injection (Amgen) and methotrexate LPF® sodium (methotrexate sodium injection) (Amgen). Methotrexate is also available from Pharmacochemie (Netherlands). Methotrexate prodrugs, homologues and/or analogues (e.g., folate antagonists) may also be used in the methods and compositions of the present invention. Alternatively, other immunosuppressive agents (or drugs that suppress the immune system) may be used in the methods and compositions of the present invention.

Suitable steroids, include but are not limited to corticosteroids or glucocorticoids, such as prednisolone (Prelone), prednisone (Delatasone), Betamethasone (Celestone), Betamethasone dipropionate, Betamethasone dipropionate, Betamethasone valerate, Cortisone (Cortone), Dexamethasone (Decadron), Hydrocortisone (Cortef), Methylprednisolone (Medrol), methylprednisolone aceponate, Budesonide (Entocort EC), Clobetasol propionate, Clobetasone butyrate, Diflucortolone valerate, Fluticasone valerate, Hydrocortisone 17-butyrate, Mometasone furoate, Aclometasone dipropionate, Fluocinolone acetonide, Triamcinolone acetonide, and combinations thereof.

Other TNF-α modulating drugs include but are not limited to etanercept (Enbrel® Immunex/Amgen), infliximab (Remicade®, J&J), anakinra (Kineret®, Aventis), adalimumab (Humira®, Abbott Labs), rituxumab (Rituxan®, Ides/Genentech), CDP870 (humanized monoclonal Fab, Pfizer), rTNFbp, rhTBP-1 (soluble p55 TNF receptor, Serono), HuMax-IL15 (anti-IL-15 monoclonal antibody, Immunex/Genmab), J695 (anti-IL-12 fully human monoclonal antibody, CAT/Wyeth), IL-1 Hy1 (Hyseq), PEGsTNF (PEGylated truncated p55 TNF receptor, Amgen), roflumilast (Altana), MRA (AntilL-6 receptor humanized monoclonal antibody, Chugai), CDP484 (anti-IL-1 antibody Fab fragment, Celltech), CNT0148 (monoclonal antibody, Centocor/Pfizer), anti-IL-18 binding protein (Serono), HuMax-CD4 (anti-CD4 human monoclonal antibody, Genmab), VX-740 (small molecule inhibitor of IL-1 converting enzyme (ICE), Aventis/Vertex), BMS-561392, DPC333 (small molecule inhibitor of TNF-α converting enzyme (TACE), BMS), Thalidomide (Celgene), Kinase inhibitors (e.g., MAP (mitogenactivated protein) kinases such as VX-745, a p38 MAP Kinase inhibitor, Vertex/Kissei) Phosphodiesterase type IV (PDEIV) inhibitors, synthetic inhibitors of HC gp-39, inosine monophosphate dehydrogenase (IMPDH) inhibitors, and combinations thereof.

Certain agents are used in the cases of refractory RA or if there are sever extraarticular complications. These drugs include cyclophosphamide, chlorambucil and cyclosporin A. Refractory RA may also include failure on any of the above therapeutic agents. The TNF variants of the present invention may also be employed for refractory RA, alone or in combination with any of the above discussed agents.

Useful Therapeutic agents for psoriasis include but are not limited to anthralin, chrysarobin, corticosteroids (steroids and glucocorticosteroids), calcipotriene, vitamin D (synthetic or natural), Tazarotene, derivatives of Vitamin A including retenoids, beta carotene, etc., methotrexate (discussed above with regard to RA), cyclosporine, acitretin, alefacept, etanercept, efalizumab. In addition, phototherapy (ultraviolet (UV) light treatment), including UVB phototherapy (both broadband and narrowband), UVA phototherapy and Eximer laser therapy, may be used in combination with the present invention. In addition to phototherapy and the TNF variants of the present invention, may also be combined with psoralen, tazarotene or anthralin. In particular, UVA light therapy, psoralen and the TNF variants of the present invention are a preferred combination.

Useful Therapeutic agents for Crohn's Disease include but are not limited to anti-inflammatory medication, incuding sulfasalazine, mesalamine (5-ASA agents) containing medications, and corticosteroids (discussed supra). In addition, certain TNF-α modulating drugs discussed supra may also be used, in particular infliximab (Remicade®, Centocor, J&J). Antibiotics may also be used to help heal abscesses, fistulas, treat bacterial growth in the intestine caused by obstruction or abscesses. In particular, metronidazole and ciprofloxacin are preferred. Anti-diarrheal medications may also be used to control diarrhea. Suitable anti-diarrheal medications include Imodium® AD (McNeil) loperamide, codeine, hydrocodone, oxycodone and fiber powders, pills or capsules. Nutritional supplements may be used to replace lost nutrients (e.g., iron, calcium, minerals, vitamins) due to diarrhea or lost appetite. Surgery may also be employed if the treatments discussed here do not control the symptoms. Generally surgery may be employed to close fistulas or remove part of the intestine where the inflammation is severe. Since surgery is many times only a temporary solution, use of the TNF variants of the present invention may enhance remission time.

Administration

'96] TNF antagonists, methotrexate and the compositions of the present invention may be administered to an individual in a variety of ways. The routes of administration include intradermal, transdermal (e.g., in slow release polymers), intramuscular, intraperitoneal, intravenous, subcutaneous, oral, topical, epidural, buccal, rectal, vaginal and intranasal routes. Any other therapeutically efficacious route of administration can be used, for example, infusion or bolus injection, absorption through epithelial or mucocutaneous linings, or by gene therapy wherein a DNA molecule encoding the therapeutic protein or peptide is administered to the patient, e.g., via a vector, which causes the protein or peptide to be expressed and secreted at therapeutic levels in vivo. In addition, the TNF antagonists, methotrexate and compositions of the present invention can be administered together with other components of biologically active agents, such as pharmaceutically acceptable surfactants (e.g., glycerides), excipients (e.g., lactose), carriers, diluents and vehicles. If desired, certain sweetening, flavoring and/or coloring agents can also be added.

The TNF antagonists and methotrexate can be administered prophylactically or therapeutically to an individual. TNF antagonists can be administered prior to, simultaneously with (in the same or different compositions) or sequentially with the administration of methotrexate. For example, TNF antagonists can be administered as adjunctive and/or concomitant therapy to methotrexate therapy.

For parenteral (e.g., intravenous, subcutaneous, intramuscular) administration, TNF antagonists, methotrexate and the compositions of the present invention can be formulated as a solution, suspension, emulsion or lyophilized powder in association with a pharmaceutically acceptable parenteral vehicle. Examples of such vehicles are water, saline, Ringer's solution, dextrose solution, and 5% human serum albumin. Liposomes and nonaqueous vehicles such as fixed oils can also be used. The vehicle or lyophilized powder can contain additives that maintain isotonicity (e.g., sodium chloride, mannitol) and chemical stability (e.g., buffers and preservatives). The formulation is sterilized by commonly used techniques. Suitable pharmaceutical carriers are described in Remington's Pharmaceutical Sciences, A. Osol, a standard reference text in this field of art. For example, a parenteral composition suitable for administration by injection is prepared by dissolving 1.5% by weight of active ingredient in 0.9% sodium chloride solution.

TNF variants of the present invention and methotrexate are administered in therapeutically effective amounts; the compositions of the present invention are administered in a therapeutically effective amount. As used herein, a “therapeutically effective amount” is such that administration of TNF antagonist and methotrexate, or administration of a composition of the present invention, results in inhibition of the biological activity of TNF relative to the biological activity of TNF when therapeutically effective amounts of antagonist and methotrexate are not administered, or relative to the biological activity of TNF when a therapeutically effective amount of the composition is not administered. A therapeutically effective amount is preferably an amount of TNF antagonist and methotrexate necessary to significantly reduce or eliminate signs and symptoms associated with a particular TNF-mediated disease. As used herein, a therapeutically effective amount is not necessarily an amount such that administration of the TNF antagonist alone, or administration of methotrexate alone, must necessarily result in inhibition of the biological activity of TNF.

Once a therapeutically effective amount has been administered, a maintenance amount of TNF antagonist alone, of methotrexate alone, or of a combination of TNF antagonist and methotrexate can be administered to the individual. A maintenance amount is the amount of TNF antagonist, methotrexate, or combination of TNF antagonist and methotrexate necessary to maintain the reduction or elimination of the signs and symptoms associated with a particular TNF-mediated disease achieved by the therapeutically effective dose. The maintenance amount can be administered in the form of a single dose, or a series or doses separated by intervals of days or weeks.

The dosage administered to an individual will vary depending upon a variety of factors, including the pharmacodynamic characteristics of the particular antagonists, and its mode and route of administration; size, age, sex, health, body weight and diet of the recipient; nature and extent of symptoms of the disease being treated, kind of concurrent treatment, frequency of treatment, and the effect desired. In vitro and in vivo methods of determining the inhibition of TNF in an individual are well known to those of skill in the art. Such in vitro assays can include a TNF cytotoxicity assay (e.g., the WEHI assay or a radioimmunoassay, ELISA). In vivo methods can include rodent lethality assays and/or primate pathology model systems (Mathison et al., J. Clin. Invest., 81:1925-1937 (1988); Beutler et al., Science 229:869-871 (1985); Tracey et al., Nature 330:662-664 (1987); Shimamoto et al., Imunol. Lett. 17:311-318 (1988); Silva et al., J. Infect. Dis. 162:421-427 (1990); Opal et al., J. Infect. Dis. 161:1148-1152 (1990); Hinshaw et al., Circ. Shock 30:279-292 (1990), all entirely incorporated by reference).

TNF variants and methotrexate can each be administered in single or multiple doses depending upon factors such as nature and extent of symptoms, kind of concurrent treatment and the effect desired. Thus, other therapeutic regimens or agents (e.g., multiple drug regimens) can be used in combination with the therapeutic co-administration of TNF antagonists and methotrexate. In a particular embodiment, a TNF antagonist is administered in multiple doses. In another embodiment, methotrexate is administered in the form of a series of low doses separated by intervals of days or weeks. Adjustment and manipulation of established dosage ranges are well within the ability of those skilled in the art.

Usually a daily dosage of active ingredient can be about 0.01 to 100 milligrams per kilogram of body weight. Ordinarily 0.1 to 40 milligrams per kilogram per day given in divided doses 1 to 6 times a day or in sustained release form is effective to obtain desired results. Second or subsequent administrations can be administered at a dosage which is the same, less than or greater than the initial or previous dose administered to the individual.

A second or subsequent administration is preferably during or immediately prior to relapse or a flare-up of the disease or symptoms of the disease. For example, second and subsequent administrations can be given between about one day to 30 weeks from the previous administration. Two, three, four or more total administrations can be delivered to the individual, as needed.

Dosage forms (composition) suitable for internal administration generally contain from about 0.1 milligram to about 500 milligrams of active ingredient per unit. In these pharmaceutical compositions the active ingredient will ordinarily be present in an amount of about 0.5-95% by weight based on the total weight of the composition.

In one embodiment provided herein, antibodies, including but not limited to monoclonal and polyclonal antibodies, are raised against variant TNF-α proteins using methods known in the art. In a preferred embodiment, these anti-TNF-α antibodies are used for immunotherapy. Thus, methods of immunotherapy are provided. By “immunotherapy” is meant treatment of an TNF-α related disorders with an antibody raised against a variant TNF-α protein. As used herein, immunotherapy can be passive or active. Passive immunotherapy, as defined herein, is the passive transfer of antibody to a recipient (patient). Active immunization is the induction of antibody and/or T-cell responses in a recipient (patient). Induction of an immune response can be the consequence of providing the recipient with a variant TNF-α protein antigen to which antibodies are raised. As appreciated by one of ordinary skill in the art, the variant TNF-α protein antigen may be provided by injecting a variant TNFa polypeptide against which antibodies are desired to be raised into a recipient, or contacting the recipient with a variant TNF-α protein encoding nucleic acid, capable of expressing the variant TNF-α protein antigen, under conditions for expression of the variant TNF-α protein antigen.

In another preferred embodiment, a therapeutic compound is conjugated to an antibody, preferably an anti-variant TNF-α protein antibody. The therapeutic compound may be a cytotoxic agent. In this method, targeting the cytotoxic agent to tumor tissue or cells, results in a reduction in the number of afflicted cells, thereby reducing symptoms associated with cancer, and variant TNF-α protein related disorders. Cytotoxic agents are numerous and varied and include, but are not limited to, cytotoxic drugs or toxins or active fragments of such toxins. Suitable toxins and their corresponding fragments include diphtheria A chain, exotoxin A chain, ricin A chain, abrin A chain, curcin, crotin, phenomycin, enomycin and the like. Cytotoxic agents also include radiochemicals made by conjugating radioisotopes to antibodies raised against cell cycle proteins, or binding of a radionuclide to a chelating agent that has been covalently attached to the antibody.

In a preferred embodiment, variant TNF-α proteins are administered as therapeutic agents, and can be formulated as outlined above. Similarly, variant TNF-α genes (including both the full-length sequence, partial sequences, or regulatory sequences of the variant TNF-α coding regions) may be administered in gene therapy applications, as is known in the art. These variant TNF-α genes can include antisense applications, either as gene therapy (i.e. for incorporation into the genome) or as antisense compositions, as will be appreciated by those in the art.

In a preferred embodiment, the nucleic acid encoding the variant TNF-α proteins may also be used in gene therapy. In gene therapy applications, genes are introduced into cells in order to achieve in vivo synthesis of a therapeutically effective genetic product, for example for replacement of a defective gene. “Gene therapy” includes both conventional gene therapy where a lasting effect is achieved by a single treatment, and the administration of gene therapeutic agents, which involves the one time or repeated administration of a therapeutically effective DNA or mRNA. Antisense RNAs and DNAs can be used as therapeutic agents for blocking the expression of certain genes in vivo. It has already been shown that short antisense oligonucleotides can be imported into cells where they act as inhibitors, despite their low intracellular concentrations caused by their restricted uptake by the cell membrane. [Zamecnik et al., Proc. Natl. Acad. Sci. U.S.A. 83:4143-4146 (1986), incorporated by reference]. The oligonucleotides can be modified to enhance their uptake, e.g. by substituting their negatively charged phosphodiester groups by uncharged groups.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat proteinliposome mediated transfection [Dzau et al., Trends in Biotechnology 11:205-210 (1993), incorporated by reference]. In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262:4429-4432 (1987); and Wagner et al., Proc. Natl. Acad. Sci. U.S.A. 87:3410-3414 (1990), both incorporated by reference. For review of gene marking and gene therapy protocols see Anderson et al., Science 256:808-813 (1992), incorporated by reference.

In a preferred embodiment, variant TNF-α genes are administered as DNA vaccines, either single genes or combinations of variant TNF-α genes. Naked DNA vaccines are generally known in the art. Brower, Nature Biotechnology, 16:1304-1305 (1998). Methods for the use of genes as DNA vaccines are well known to one of ordinary skill in the art, and include placing a variant TNF-α gene or portion of a variant TNF-α gene under the control of a promoter for expression in a patient in need of treatment. The variant TNF-α gene used for DNA vaccines can encode full-length variant TNF-α proteins, but more preferably encodes portions of the variant TNF-α proteins including peptides derived from the variant TNF-α protein. In a preferred embodiment a patient is immunized with a DNA vaccine comprising a plurality of nucleotide sequences derived from a variant TNF-α gene. Similarly, it is possible to immunize a patient with a plurality of variant TNF-α genes or portions thereof as defined herein. Without being bound by theory, expression of the polypeptide encoded by the DNA vaccine, cytotoxic T-cells, helper T-cells and antibodies are induced which recognize and destroy or eliminate cells expressing TNF-α proteins.

Therapeutic Uses

Once made, the variant TNF-α proteins and nucleic acids of the invention find use in a number of applications. In a preferred embodiment, the variant TNF-α proteins are administered to a patient to treat a TNF-α related disorder. By “TNF-α related disorder” or “TNF-α responsive disorder” or “condition” herein is meant a disorder that may be ameliorated by the administration of a pharmaceutical composition comprising a variant TNF-α protein, including, but not limited to, neurologic, pain, pulmonary, hematological, oncology, fibrotic, inflammatory and immunological disorders. The variant TNF-α protein may also be used to reduce toxicities associated with agents that mediate TNF such as interleukin-2. The variant TNF-α is a major effector and regulatory cytokine with a pleiotropic role in the pathogenesis of diseases, including immune-regulated diseases, fibrosis conditions, oncological conditions, and inflammation related conditions. In a preferred embodiment, the variant TNF-α protein is used to treat arthritis, psoriatic arthritis, ankylosing spondylitis, spondyloarthritis, spondyloarthropathies, rheumatoid arthritis, juvenile rheumatoid arthritis, juvenile idiopathic arthritis, reactive arthritis (Reiter Syndrome) scleroderma, Sjogren's syndrome, keratoconjunctivitis, keratoconjunctivitis sicca, TNF-receptor associated periodic syndrome (TRAPS), periodic fever, periprosthetic osteolysis, apthous stomatitis, pyoderma gangrenosum, uveitis, reticulohistiocytosis, inflammatory bowel diseases, sepsis and septic shock, Crohn's Disease, psoriasis, autoimmune thyroiditis, dermatitis, atopic dermatitis, eczematous dermatitis)graft versus host disease (GVHD), hematologic malignancies, such as multiple myeloma (MM), refractory MM, Waldenstrom's macroglobulinemia, myelodysplastic syndrome (MDS) acute myelogenous leukemia (AML); solid tumor malignancies, such as ovarian carcinoma, melanoma, renal cell carcinoma; and the inflammation associated with tumors, pain, including spinal disk pain, chronic lower back pain chronic neck pain, pain due to bone metastasis, pain and swelling after molar extraction, neurological conditions and neural damage conditions such as peripheral nerve injury, demyelinating diseases, adrenoleukodystrophy, X-linked adrenoleukodystrophy (X-ALD), the childhood cerebral form (CCER) and the adult form, adrenomyeloneuropathy (AMN), adrenoleukodystrophy, sciatica, autoimmune sensorineural hearing loss, chronic inflammatory demyelinating polyneuropathy (CIDP), Alzheimers disease, Parkinson's disease, diabetes, insulin resistance, insulin sensitivity, Syndrome X, Wegener's Granulomatosis, dermatomyositis, histicytosis, polymyositis, cancer cachexia, temporomandibular disorders, refractory ocular sarcoidosis, sarcoidosis, behcet's, churg-strauss syndrome, asthma, idiopatic pneumonia following bone marrow transplantation, systemic lupus erythematosus (SLE), lupus nephritis, multiple sclerosis (MS), amyotrophic lateral sclerosis (ALS) myasthenia gravis, atherosclerosis, polyneuropathy, orangomegaly, endocrinopathy, M protein, skin changes (POEMS syndrome), Sneddon-Wilkinson disease, necrotizing crescentic glomerulonephritis, renal amyloidosis, AA amyloidosis, erythema nodosum leprosum (ENL), chronic kidney disease, malnutrition, inflammation and atherosclerosis (MIA) syndrome, chronic obstructive pulmonary disease (COPD), pulmonary fibrosis, endometriosis, idiopathic thrombocytopenic purpura (ITP), AIDS, HIV disease and related conditions, including tuberculosis (TB) in AIDS patients, inflammation and cancer (e.g. Kaposi's Sarcoma, HIV retinopathy, uveitis, P jiroveci pneumonia (PCP), Pneumocystis choroiditis, HIV-associated lymphoma), alopecia areata, allergic responses due to arthropod bite reactions, aphthous ulcer, iritis, conjunctivitis, keratoconjunctivitis, ulcerative colitis, asthma, allergic asthma, cutaneous lupus erythematosus, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens Johnson syndrome, idiopathic sprue, lichen planus, Graves ophthalmopathy, sarcoidosis, primary biliary cirrhosis, and interstitial lung fibrosis. See for example, Tsimberidou et al., Expert Rev Anticancer Ther 2002 June;2(3):277-86, U.S. Pat. No. 6,015,557; Present, D H, et al., N Engl J Med 1999 340: 1398-1405; Braun, Jet al Lnacet 2002; 359: 1187-93; Williams, J D and Griffiths, C E, Clin Exp Dermatol. 2002 October; 27(7) 585-90. Chin, R L et la, J. Neurol. Sci 2003 Jun. 15: 210(1-2) 19-21; Lovell, D J et al., N Engl. J. Med 2000; 342: 763-769; Lorenz, H M and Kalden, JR Arthritis Res. 2002; 4 Suppl 3:S17-24; Kalden, JR, Arthritis Res 2002; 27(7): 585-90; Mease, P J et al., Lancet. 2000 Jul. 29:356(9227): 385-90; Gorman, J D, et al., N Engl J Med 2000; 346: 1349-1356; Anderson, V C and Israel, Z, Curr Rev Pain 2000; 4(2): 105-111; Marshall, L L and Trethewie, E R, Lancet 291973) 320; Takahashi, H, et al., Spine, 21 (1996) 218-224; Igarashi, T, et al., Spine, 25 (2000) 2975-2980; Wagner, R and Myers, RR Neuroreport., 7 (1996) 2897-2901; Olmarker, K and Rydevik, B, Spine, 26 (2001) 863-869; Sommer, C et al., J Peripher Nery Syst, 6 (2001) 67-72; Tobinick E, Curr Med Res Opin, July 2004; 20(7) 1075-1085; Genevay, S et al., Ann Rheum Dis 2004: doi:10.1136/ard.2003.016451; Tobinick, E, Clinical Therapeutics August 2003. 25(8): 2279-88; Wu, S, et al. Cancer Res. 1993 Apr. 15; 538): 1939-44; all Baughman, R P et al., Chest. 2005 August; 128(2): 1062-47; Gherardi, r K et al., Ann Neurol 1994 April; 35(4): 501-5; Van den Bosch, F., et al., Ann Rheum Dis. 2001 Nov: 60 Suppl 3: iii33-6; Voigtlander, C, et al., Arch Dermatol. 2001 December; 137(12): 1571-4 Zaenker, M et al., Int J Tissue React. 2004; 26(3-4): 85-92; Smith, G R et al., Intern Med J. 2004 Sep-Oct; 34(9-10): 570-2; Tsimberidou, A M et al., Leuk Res 2003 May; 27(5): 375-80; Macdougall, IC Nephrol Dial Transplant. 2004 August; 19 Suppl 5:V73-78; Mcguinnessa, M C et al., J Neuroimmunology, 61(2): 161-169 (9/1995); all incorporated by reference.

Inflammatory bowel disease (“IBD”) is the term generally applied to two diseases, namely ulcerative colitis and Crohn's disease. Ulcerative colitis is a chronic inflammatory disease of unknown etiology afflicting only the large bowel and, except when very severe, limited to the bowel mucosa. The course of the disease may be continuous or relapsing, mild or severe. It is curable by total colostomy which may be needed for acute severe disease or chronic unremitting disease. Crohn's disease is also a chronic inflammatory disease of unknown etiology but, unlike ulcerative colitis, it can affect any part of the bowel. Although lesions may start superficially, the inflammatory process extends through the bowel wall to the draining lymph nodes. As with ulcerative colitis, the course of the disease may be continuous or relapsing, mild or severe but, unlike ulcerative colitis, it is not curable by resection of the involved segment of bowel. Most patients with Crohn's disease come to surgery at some time, but subsequent relapse is common and continuous medical treatment is usual. Remicade® (inflixmab) is the commercially available treatment for Crohn's disease. Remicade® is a chimeric monoclonal antibody that binds to TNF-α. The use of the TNF-α variants of the present invention may also be used to treat the conditions associated with IBD or Crohn's Disease.

“Sepsis” is herein defined to mean a disease resulting from gram positive or gram negative bacterial infection, the latter primarily due to the bacterial endotoxin, lipopolysaccharide (LPS). It can be induced by at least the six major gram-negative bacilli and these are Pseudomonas aeruginosa, Escherichia coli, Proteus, Klebsiella, Enterobacter and Serratia. Septic shock is a condition which may be associated with Gram positive infections, such as those due to pneumococci and streptococci, or with Gram negative infections, such as those due to Escherichia coli, Klebsiella-Enterobacter, Pseudomonas, and Serratia. In the case of the Gram-negative organisms the shock syndrome is not due to bloodstream invasion with bacteria per se but is related to release of endotoxin, the LPS moiety of the organisms' cell walls, into the circulation. Septic shock is characterized by inadequate tissue perfusion and circulatory insufficiency, leading to insufficient oxygen supply to tissues, hypotension, tachycardia, tachypnea, fever and oliguria. Septic shock occurs because bacterial products, principally LPS, react with cell membranes and components of the coagulation, complement, fibrinolytic, bradykinin and immune systems to activate coagulation, injure cells and alter blood flow, especially in the microvasculature. Microorganisms frequently activate the classic complement pathway, and endotoxin activates the alternate pathway.

The TNF-α variants of the present invention effectively antagonize the effects of wild type TNF-αinduced cytotoxicity and interfere with the conversion of TNF into a mature TNF molecule (e.g., the trimer form of TNF). Thus, administration of the TNF variants can ameliorate or eliminate the effects of sepsis or septic shock, as well as inhibit the pathways associated with sepsis or septic shock. Administration may be therapeutic or prophylactic. The TNF-α variants of the present invention effectively antagonize the effects of wild type TNF-α-induced cytotoxicity in cell based assays and animal models of peripheral nerve injury and axonal demyelination/degeneration to reduce the inflammatory component of the injury or demyelinating insult. This is believed to critically contribute to the neuropathological and behavioral sequelae and influence the pathogenesis of painful neuropathies.

Severe nerve injury induces activation of Matrix Metallo Proteinases (MMPs), including TACE, the TNF-α-converting enzyme, resulting in elevated levels of TNF-α protein at an early time point in the cascade of events that leads up to Wallerian nerve degeneration and increased pain sensation (hyperalgesia). The TNF-α variants of the present invention antagonize the activity of these elevated levels of TNF-α at the site of peripheral nerve injury with the intent of reducing macrophage recruitment from the periphery without negatively affecting remyelination. Thus, reduction of local TNF-induced inflammation with these TNF-α variants would represent a therapeutic strategy in the treatment of the inflammatory demyelination and axonal degeneration in peripheral nerve injury as well as the chronic hyperalgesia characteristic of neuropathic pain states that often results from such peripheral nerve injuries. Intraneural administration of exogenous TNF-α produces inflammatory vascular changes within the lining of peripheral nerves (endoneurium) together with demyelination and axonal degeneration (Redford et al 1995). After nerve transection, TNF-positive macrophages can be found within degenerating fibers and are believed to be involved in myelin degradation after axotomy (Stoll et al 1993). Furthermore, peripheral nerve glia (Schwann cells) and endothelial cells produce extraordinary amounts of TNF-α at the site of nerve injury (Wagner et al 1996) and intraperitoneal application of anti-TNF antibody significantly reduces the degree of inflammatory demyelination strongly implicating a pathogenic role for TNF-α in nerve demyelination and degeneration (Stoll et al., 1993). Thus, administration of an effective amount of the TNF-α variants of the present invention may be used to treat these peripheral nerve injury or demyelinating conditions, as well as Alzheimers disease and Parkinson's disease.

As used herein, “fibrosis” refers to fibrous tissue formed in fibrotic diseases; e.g., the formation of fibrous tissue as a reparative, or reactive process, as opposed to formation of fibrous tissue as a normal constituent of an organ or tissue, wherein fibrous tissue refers to tissue composed of or containing fibroblasts, and also the fibrils and fibers of connective tissue formed by such cells. As used herein, “fibrosis” or any grammatical equivalents means one or more types of fibrosis including, but not limited to endomyocardial, peritoneal, intrarenal, intraorgan, glomerulonephritis, subcutaneous, intraarterial, idiopathic retroperitoneal, leptomeningeal, mediastinal, nodular subepidermal, pericentral, perimuscular, pipestem, replacement, or subadventitial fibrosis. Fibrosis may occur as a result of a disease condition such as those listed above, or it can develop in response to medical procedures such as, for example, the implantation of a cellular encapsulation device, a cardiac stent, a cancer stent, a euglycemic clamp, or an artificial heart valve, or following an interventional procedure such as cardiac catheterization, or carotid endarterectomy, or it can develop following any surgical procedure, or traumatic event which causes tissue damage.

In a preferred embodiment, a therapeutically effective dose of a variant TNF-α protein is administered to a patient in need of treatment. By “therapeutically effective dose” herein is meant a dose that produces the effects for which it is administered. The exact dose will depend on the purpose of the treatment, and will be ascertainable by one skilled in the art using known techniques. In a preferred embodiment, dosages of about 5 μg/kg are used, administered either intravenously or subcutaneously. As is known in the art, adjustments for variant TNF-α protein degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

In a preferred embodiment, the DNA vaccines include a gene encoding an adjuvant molecule with the DNA vaccine. Such adjuvant molecules include cytokines that increase the immunogenic response to the variant TNF-α polypeptide encoded by the DNA vaccine. Additional or alternative adjuvants are known to those of ordinary skill in the art and find use in the invention.

All references cited herein, including patents, patent applications (provisional, utility and PCT), and publications are incorporated by reference.

### EXAMPLES

### Example 1

### Clinical Treatment of Rheumatoid Arthritis by Multiple Infusions of TNF-α Variant with and Without Methotrexate (MTX)

A randomized, double-blind, placebo controlled study is conducted to evaluate the safety and efficacy of a TNF-α variant (XPro™ 1595) following multiple infusions of 1, 3 or 10 mg/kg, alone or in combination with methotrexate, compared to multiple infusions of placebo in combination with methotrexate, in the treatment of rheumatoid arthritis (RA) in patients.

A statistically relevant amount of patients who had been using methotrexate for at least 6 months, had been on a stable dose of 7.5 mg/wk for at least 4 weeks, and had active disease (according to the criteria of the American College of Rheumatology) with erosive changes on X-rays of hands and feet, are enrolled in the trial. Active disease is defined by the presence of six or more swollen joints plus at least three of four secondary criteria (duration of morning stiffness about 45 minutes; tender or painful joints; erythrocyte sedimentation rate (ESR) of at least about 28 mm/hour; C-reactive protein (CRP) of at least about 220 mg/1. In patients using corticosteroids (less than 7.5 mg/day) or non-steroidal anti-inflammatory drugs (NSAIDs), the doses are stable for 4 weeks prior to screening. The dose of corticosteroids and/or NSAIDs should remain stable throughout trial participation.

XPro™ 1595 is supplied as a sterile solution containing 10 mM histidine, 150 mM NaCl, 0.01% (w/v) Polysorbate 20, pH 6.5. The placebo vials contain 0.1% human serum albumin in the same buffer. Before use, the appropriate amount of XPro™ 1595 or placebo is diluted to 300 ml in sterile saline by a pharmacist, and administered intravenously via an in-line filter over 2 hours. The characteristics of the placebo and XPro™ 1595 infusion bags are identical, and the investigators and patients do not know which infusion is being administered.

Patients are randomized to one of seven treatment groups. Each of the patients received multiple infusions of either 0, 1, 3 or 10 mg/kg XPro™ 1595. Infusions are administered at weeks 0, 2, 6, 10 and 14. Starting at week 0, the patients are receiving 7.5 mg/wk of methotrexate or 3 placebo tablets/week. Patients are monitored for adverse events during infusions and regularly thereafter, by interviews, physical examination, and laboratory testing.

The six primary disease-activity assessments are chosen to allow analysis of the response in individual patients according to the Paulus index (Paulus, et al., Arthritis Rheumatism 33:477-484 (1990), the teachings of which are incorporated herein by reference). The assessments contributing to this index are the tender joint and swollen joint scores (60 and 58 joints, respectively, hips not assessed for swelling; graded 0-3), the duration of morning stiffness (minutes), the patient's and physician's assessment of disease severity (on a 5-point scale, ranging from 1 (symptom-free) to 5 (very severe), and erythrocyte sedimentation rate (ESR). Patients are considered to have responded if at least four of the six variables improved, defined as at least 20% improvement in the continuous variables, and at least two grades of improvement or improvement from grade 2 to 1 in the two disease-severity assessments (Paulus 20% response). Improvements of at least 50% in the continuous variables were also used (Paulus 50% response).

Other disease-activity assessments include the pain score (0-10 cm on a visual analogue scale (VAS)), an assessment of fatigue (0-10 cm VAS), and grip strength (0-300 mm Hg, mean of three measurements per hand by sphygmomanometer cuff). The ESR is measured at each study site with a standard method (Westergen). C-reactive protein (CRP) was measured by rate nephelometry (Abbott fluorescent polarizing immunoassay). See also, Elliott et al., Lancet 344:1105-1110 (1994); Elliott et al., Lancet 344:1125-1127 (1994); and Elliott et al., Arthritis Rheum. 36(12):1681-1690 (1993), which references are entirely incorporated herein by reference. Evaluations are performed at weeks 1, 2, 4, 6, 8, 10, 12, 14, 16, 18, 20, 22 and 24. The pre-specified primary analysis in this trial is the comparison of the total time of clinical response during the 24-week follow-up period.

It is possible that patients may experience infusion-related reactions with retreatment. These infusion-related reactions include headache, fever, facial flushing, pruritus, myalgia, nausea, chest tightness, dyspnea, vomiting, erythema, abdominal discomfort, diaphoresis, shivers, hypertension, lightheadedness, hypotension, palpitations and somnolence. These hypersensitivity reactions, as described herein, may occur whenever protein-containing materials, such as XPro™1595, are administered. Thus, it is unclear whether these symptoms represent an immunologic event or physical factors such as infusion rate and immunoglobulin aggregation. Investigators have reported that symptoms resolve in some patients by decreasing the rate of the infusion. Previous literature reports indicate that vasomotor symptoms have been observed in patients receiving intravenous immunoglobulin therapy (Berkman et al., Ann. Intern Med. 112:278-292 (1990); Ochs et al., Lancet 2:1158-1159 (1980)).

### Example 2

### Clinical Treatment of Rheumatoid Arthritis by Single Infusion of a TNF-α Variant in Patients Receiving Methotrexate

A randomized, double-blind, placebo controlled study is conducted to evaluate the effects of a single infusion of placebo, 5,10 or 20 mg/kg XPro™1595 in combination with methotrexate, administered at a dose of 10 mg/week, in the treatment of rheumatoid arthritis (RA) in patients. RA patients who have received about three months therapy with methotrexate administered at a stable dose of 10 mg/wk for at least 4 weeks prior to screening, and who still had active disease according to the criteria of the American College of Rheumatology, are enrolled in the study. Active disease was defined by the presence of six or more swollen joints plus at least three of four secondary criteria (duration of morning stiffness of about 45 minutes; about 6 tender or painful joints; ESR of at least about 28 mm/hour; C-reactive protein (CRP) 20 mg/1. Patients taking NSAIDs and corticosteroids (prednisone) at screening were allowed to continue at stable doses (7.5 mg/day).

The XPro™ 1595 is supplied as identified and assessed as decribed above. The primary measurement of clinical response is defined by the ACR preliminary definition of response (Felson et al., Arthritis Rheumatism 38(6):727-735 (1995)). Patients are considered to have a response if they had a 20% reduction in swollen and tender joint count, and had experienced a 20% reduction in 3 of the 5 following assessments: patient's assessment of pain (VAS), patient's global assessment of disease activity (VAS), physician's global assessment of disease activity (VAS), patient's assessment of physical function (HAQ), and an acute phase reactant (ESR). The ESR was measured at each study site with a standard method (Westergen). Evaluations are performed at day 3, and at weeks 1, 2, 4, 6, 8, 10, and 12.

### Example 3

### Clinical Treatment of Rheumatoid Arthritis by Repeated Dose Administration of a TNF-α Variant in Patients Following a Single Dose Double-Blind Placebo-Controlled Trial

An open label study is conducted to evaluate the effects of repeated infusions of 10 mg/kg XPro™ 1595 in combination with methotrexate, administered at a dose of 10 mg/week, in the treatment of rheumatoid arthritis in patients. As described in Example 2, a randomized, double-blind, placebo controlled, 12 week study of XPro™1595 is conducted in RA patients who had active disease despite receiving three months therapy with methotrexate administered at a stable dose of 10 mg/wk for at least 4 weeks prior to screening. At week 12, patients who complete the 12 week evaluation period and have not experienced adverse events prohibiting further infusions of XPro™1595, are offered 3 subsequent open label infusions of XPro™1595, administered at a dose of 10 mg/kg, at eight week intervals (weeks 12, 20, 28). Patients entering this open label study are followed up to 40 weeks after initial entry. The primary measurement of clinical response is defined by the ACR preliminary definition of response (Felson et al., Arthritis Rheumatism 38(6):727-735 (1995)). Patients are considered to have a response if they have a 20% reduction in swollen and tender joint count, and had experienced a 20% reduction in 3 of the 5 following assessments: patient's assessment of pain (VAS), patient's global assessment of disease activity (VAS), physician's global assessment of disease activity (VAS), patient's assessment of physical function (HAQ), and an acute phase reactant (ESR). The ESR is measured at each study site with a standard method (Westergen).

### Example 4

### Single Dose of XPro™ 1595 with MTX and Folate

Twenty-four (24) patients are enrolled in a single dose study multi-center, open label, single dose, sequential dose-escalation trial (0.1, 0.3, 1 and 3 mg/kg) of XPronˆ1595 with Methotrexate (MTX) and folate in four cohorts of six RA patients. The treatment period is one day and the patients are monitored for 31 days. Dose escalation proceeds after all six patients in the preceding cohort have been observed for two weeks.

### Example 5

### Weekly Dose of XPro™ 1595 with MTX and Folate

12 weekly doses of XPro™ 1595 with MTX and folate. Study medication is administered by SC injection to cohorts of six patients in ascending doses of 0.1, 0.3, 1.0 and 3 mg/kg. Study medication will be administered on days 1, 8, 15, 22, 29, 36, 43, 50, 57, 64, 71, and 78. If no MTD is noted, the sponsor may further expand one cohort with up to 12 additional patients at the anticipated biologically active dose to obtain additional safety and PK data. Days 1-78 will be considered the treatment period; follow-up will continue to day 115.

A minimum of six patients will be entered into each opened dose level cohort. The first cohort of patients will receive a single dose of 0.1 mg/kg XPro™1595. Dose levels will be increased in successive increments according to the following dose escalation scheme: 0.1, 0.3, 1, 3 mg/kg. Dose escalation will continue until an MTD has been identified or a dose of 3 mg/kg has been reached, whichever comes first. With respect to stopping rules, dose escalation may occur if no patient within a six-patient cohort experiences a DLT. If one of six patients within a cohort experiences a DLT, an additional three patients will be treated at the same dose level. If one of nine patients within a dose level experiences a DLT, dose escalation may continue. If two of six or two of nine patients within a dose level experience a DLT, that dose level will be defined as exceeding MTD; no further dose escalation will occur. The previous dose level will be considered the MTD and may be expanded up to 12 patients to further elucidate the Adverse Events (AEs) and to obtain additional blood samples for PK analysis.

Clinical disease activity comprising variables of SJC/TJC, morning stiffness, ESR, CRP, patient's and investigator's global assessment of disease activity [by disease activity score (DAS) and Health Activity Questionnaire (HAQ)], will be assessed at baseline and days 29, 50, 78, 92, and 115. The primary efficacy assessment will be the ACR20 score on day 92. The biologic effects of XPro™ 1595 will be assessed by measurements of CRP, ESR, and RF at baseline and days 29, 50, 78 and 115.

### Example 6

### Juvenile RA (JRA)

Approximately 60 to 70 patients will be treated for 90 days using the JRA Definition of Improvement (DOI) at 90 days with a dosage of about 0.1 to 5 mg/kg. The DOI is defined as a thirty percent (30%) improvement of 3 of the following 6 indicators: number of active joints, number of joints LOM, funcational assessment, physician's global, patient/parent global. Alternatively an endpoint of the number of flares in a defined period may be used.

### Example 7

### Psoriatic Arthritis

Approximately 200 to 325 patients will be treated using as an endpoint ACR20. The time frame for the treatment will be approximately 10 to 15 weeks. The patients will be evaluated for PASI (% BSA, induration, erythema, and scaling).

### Example 8

### Psoriasis

Patients will be treated for 3 and/or 6 months then have randomized discontinuation. Goals for treatment will be reduction in PASI (% BSA, induration, erythema, scaling) by approximately 75%. The TNF of the present invention may be administered subcutaneously or intravenously or intramuscularly on at least a weekly basis. Evaluation of the treatment will occur at 12 to 14 weeks and at the end of the trial period.

### Example 9

### Ankylosino Spondylitis

Patients will be administered the TNF-α variants of the present invention for a period of 24 weeks. The endpoints will be ASAS and a goal of improvement of more than 20% in and absolute improveoment of more than 10 U in 3 of the following: patient global, pain function (BASF!), spinal mobility assessments, swollen joints and stiffness. In addition, there should be minimal deterioration in the remaining domain.

### Example 10

### Crohn's

Patients will be administered (subcutaneously or intravenously) the TNF variants of the present invention over a period of at least 26 weeks (approximately every 2 weeks). To qualify for the study, patients must have active disease despite other treatment (discussed supra). The clinical endpoints will be remission at the end of the study and discontinuation of steroid use. Patients may optionally be on MTX, 5-ASA, steroids or other immunomodulators during this study.

### Example 11

### Ulcerative Colitis

Patients will be administered the TNF variants of the present invention via IV administration on a every 2 week schedule until the completion of the study. The endpoints will be assayed at 8 weeks and at 30 weeks. The endpoints include a 30% decrease in the Mayo Score (stool frequency, rectal bleeding, endoscopic findings, and physician's global assessment). The goal will be to have patients score less than 2 points on the Mayo Score, with no bleeding, normal mucosa and solid stools. Another goal will be to decrease steroid use.

The codes used in the figures and experiments described herein are based upon SEQ ID NO. 23 (also referred to as “wild-type TNF-α sequence” and/or “root TNF-α sequence”):

| | |
|---|---|
| <0001 <VRSSSRTPSDKPVAHVVANPQAEGQLQ | (SEQ ID NO. 23) |
| WLNRRANALLANGVELRDNQLWPSEGLYLIYSQV | |
| LFKGQGCPSTHVLLTHTISRIAVSYQTKVNLLSA | |
| IKSPCQRETPEGAEAKPWYEPIYLGGVFQLEKGD | |
| RLSAEINRPDYLDFAESGQVYFGIIAL>0157>. | |

The codes used in the Figures and experiments above disclose the following TNF-α variants of the present invention:

| | |
|---|---|
| Name | Modifications |

| | |
|---|---|
| XENP268 | <001<-1097T-A145R->157>with MHHHHHH as N- |
| | terminal “tag” |
| XENP344 | <001<-V0001M-R0031C-p0031Peg5-00069V- |
| | Y0087H-00101A-A0145R->0157> |
| XENP345 | <001<-V001M-R031C-p031Peg5-0069V-1097T- |
| | C101A-A145R->157> |
| XENP346 | <001<-V001M-R031C-p031Peg10-0069V-1097T- |
| | C101A-AI45R->157> |
| XENP550 | <001<-V001M-R031C-0069V-Y087H-C101A- |
| | AI45R->157> |
| XENP551 | <001<-V001M-R031C-0069V-1097T-C101A- |
| | A145R->157> |
| XENP557 | <001<-V001L-R031C-0069V-Y087H-C101A- |
| | A145R->157>, with M as N-terminal “tag” |
| XENP1593 | <001<-V001M-R031C-p031Peg40-0069V-Y087H- |
| | C101A-A0145R->157> |
| XENP1594 | <001<-V001M-R031C-p031Peg20-0069V-Y087H- |
| | C101A-A0145R->157> |
| XENP1595 | <001<-V001M-R031C-p031Peg10-0069V-Y087H- |
| | C101A-A0145R->157> |

The “μ” means a modification at that position that is not an amino acid change. Thus, “R031C-μ031 Peg5” means both an amino acid change and a pegylation with 5 kD PEG at position 31.

### Example 12

### Ankylosing Spondylitis

Ankylosing spondylitis (AS) is an inflammatory disorder that primarily affects the axial skeleton; peripheral joints and extraarticular structures may also be involved. The enthesis, the site of ligamentous attachment to bone, is thought to be the primary site of pathology in AS, particularly in lesions around the pelvis and spine. Enthesitis is associated with prominent edema of the adjacent bone marrow and is often characterized by erosive lesions that eventually undergo ossification. Sacroiliitis is usually one of the earliest manifestations of AS, with features of both enthesitis and synovitis. The early lesions consist of subchondral granulation tissue containing lymphocytes, plasma cells, mast cells, macrophages, and chondrocytes; infiltrates of lymphocytes and macrophages in ligamentous and periosteal zones; and subchondral bone marrow edema. Synovitis follows and may progress to pannus formation. Islands of new bone formation can be found within the inflammatory infiltrates. Usually, the thinner iliac cartilage is eroded before the thicker sacral cartilage. The irregularly eroded, sclerotic margins of the joint are gradually replaced by fibrocartilage regeneration and then by ossification. Ultimately, the joint may be totally obliterated.

A multicenter, randomized, placebo-controlled study will assess the reduction in signs and symptoms of ankylosing spondylitis at week 24 after an induction regimen of XPro™ 1595 followed by infusions every 6 weeks in a large group of patients. A statistically relevant number of patients are randomly assigned to receive infusions of placebo or XPro™ 1595 at weeks 0, 2, 6, and every 6 weeks thereafter. Efficacy was assessed using the Ankylosing Spondylitis Assessment (ASAS) response criteria and ASAS partial remission criteria, Bath Ankylosing Spondylitis Disease Activity Index (BASDAI), Bath Ankylosing Spondylitis Functional Index (BASFI), Bath Ankylosing Spondylitis Metrology Index (BASMI), chest expansion, night pain, patient global assessment, C-reactive protein (CRP), and Short Form-36 (SF-36). The primary endpoint was the proportion of ASAS 20 responders at week 24.

### Example 13

### TNFa Inhibitor in Mouse Model for Asthma

This example describes a study using Ovalbumin (OVA)-induced Allergic Asthma Mice. The mouse OVA model of allergic asthma (Hessel, E. M., et al. (1995) Eur. J. Pharmacol. 293:401; Daphne, T., et al. (2001) Am. J. Respir. Cell Mol. Biol. 25:751, is used in the following study for treating allergic asthma.

All mice are sensitized to OVA (chicken egg albumin, crude grade V; Sigma, St. Louis, Mo.). Active sensitization is performed without an adjuvant by giving seven intraperitoneal injections of 10 pg OVA in 0.5 ml pyrogen-free saline on alternate days (one injection per day). Three weeks after the last sensitization, mice are exposed to either 16 OVA challenges (2 mg/ml in pyrogen-free saline) or 16 saline aerosol challenges for 5 min on consecutive days (one aerosol per day). An additional group of mice first receive eight. OVA aerosols, followed by eight saline aerosols (OVA/saline, spontaneous resolution group).

For the experiment in the more severe ongoing model of allergic asthma, all mice are sensitized to OVA by active sensitization with two intraperitoneal injections (7 d apart) of 0.1 ml alum-precipitated antigen, comprising 10 μg OVA adsorbed onto 2.25 mg alum (Alumlmject; Pierce, Rockford, III.). Two weeks after the second sensitization, mice are exposed to either six OVA challenges (10 mg/ml in pyrogen-free saline) or six saline aerosol challenges for 20 min every third day (one aerosol every third day). An additional group of mice first receive three OVA aerosols, followed by three saline aerosols (OVA/saline, spontaneous resolution group).

The aerosol treatment is performed in a plexiglas exposure chamber (5 liter) coupled to a Pari LC Star nebulizer (PARI Respiratory Equipment, Richmond, Va.; particle size 2.5-3.1 pm) driven by compressed air at a flow rate of 6 liters/min. Aerosol is given in groups composed of no more than eight animals.

XPro™, 1595 is administered to the OVA sensitized mice in a range of doses after the second sensitization according to standard protocols known in the art. Appropriate placebo controls are also administered.

Airway responsiveness is measured in conscious, unrestrained mice using barometric whole-body plethysmography by recording respiratory pressure curves in response to inhaled methacholine (acetyl-β-methylcholine chloride; Sigma). Briefly, mice are placed in a whole-body chamber, and basal readings are obtained and averaged for 3 min. Aerosolized saline, followed by doubling concentrations of methacholine (ranging from 1.6-50 mg/ml saline), are nebulized for 3 min, and readings are taken and averaged for 3 min after each nebulization. Dose-response curves (DRCs) to methacholine are statistically analyzed by a general linear model of repeated measurements followed by post-hoc comparison between groups. Data are LOG transformed before analysis to equalize variances in all groups.

After measurement of in vivo airway responsiveness, mice are sacrificed by intraperitoneal injection of 1 ml 10% urethane in pyrogen-free saline (Sigma). Subsequently, mice are bled by cardiac puncture, and OVA-specific IgE is measured by ELISA. Briefly, microtiter plates (Nunc A/S, Roskilde, Denmark) are coated overnight at 4° C. with 2 pg/ml rat anti-mouse IgE (clone EM95) diluted in phosphate-buffered saline (PBS). The next day, the ELISA is performed at room temperature. After blocking with ELISA buffer (PBS containing 0.5% bovine serum albumin [Sigma], 2 mM EDTA, 136.9 mM NaCl, 50 mM Tris, 0.05% Tween-20 [Merck, Whitehouse Station, N.J.] pH 7.2) for 1 h, serum samples and a duplicate standard curve (starting 1:10), diluted in ELISA buffer, are added for 2 h. An OVA-specific IgE reference standard is obtained by intraperitoneal immunization with OVA and arbitrarily assigned a value of 10,000 experimental units/ml (EU/ml). After incubation, 1 pg/ml of OVA coupled to digoxigenin (DIG), which is prepared from a kit containing DIG-3-o-methylcarbonyl-caminocaproic acid-N-hydroxy-succinimide-ester (Roche Diagnostics, Basel, Switzerland) in ELISA buffer, is added for 1.5 h, followed by incubation with anti-DIG-Fab fragments coupled to horseradish peroxidase (Roche Diagnostics) diluted 1:500 in ELISA buffer for 1 hour. Color development is performed with o-phenylenediamine-dichloride substrate (0.4 mg/ml, Sigma) and 4 mM H202 in PBS and stopped by adding 4 M H2SO4. The optical density is read at 492 nm, using a Benchmark microplate reader (Bio-Rad Laboratories, Hercules, Calif.). The detection limit of the ELISA is 0.5 EUliml IgE.

Bronchial alveolar lavage (BAL) is performed immediately after bleeding of the mice. Briefly, the airways are lavaged five times through a tracheal cannula with 1-ml aliquots of pyrogen-free saline warmed to 37° C. The recovered lavage fluid is pooled, and cells are pelleted (32×g, 4° C., 5 min) and resuspended in 150 μl cold PBS. The total number of cells in the BALF is determined using a Bürker Turk counting-chamber (Karl Hecht Assistent KG, Sondheim/Röhm, Germany). For differential BALF cell counts, cytospin preparations are made and stained with Diff-Quick (Dade AG, Düdingen, Switzerland). Per cytospin, 400 cells are counted and differentiated into mononuclear cells (monocytes, macrophages, and lymphocytes), eosinophils, and neutrophils by standard morphology. Statistical analysis is performed using the nonparametric Mann-Whitney U test.

!65] Cytokine production by antigen-restimulated T cells in lung tissue is determined as described previously (Hofstra, C. L., et al. (1999) Inflamm. Res. 48:602). Briefly, the lungs are lavaged as described above and perfused via the right ventricle with 4 ml saline containing 100 Wm1 heparin to remove any blood and intravascular leukocytes. Complete lung tissue is removed and transferred to cold sterile PBS. Lungs are then minced and digested in 3 ml RPMI 1640 containing 2.4 mg/ml collagenase A and DNase I (grade II) (both from RocheDiagnostics) for 30 min at 37° C. Collagenase activity is stopped by adding fetal calf serum (FCS). The lung tissue digest is filtered through a 70-pm nylon cell strainer (Becton Dickinson Labware, Franklin Lakes, N.J.) with 10 ml RPMI 1640 to obtain a single-cell suspension. The lung-cell suspension is washed, resuspended in culture medium (RPMI 1640 containing 10% FCS, 1% glutamax 1, and gentamicin [all from Life Technologies, Gaithersburg, Md.]) and 50 mM β-mercaptoethanol (Sigma), and the total number of lung cells is determined using a Bürker-Turk counting-chamber. Lung cells (8×10⁵ lung cells/well) are cultured in round-bottom 96-well plates (Greiner Bio-One GmbH, Kremsmuenster, Austria) in the presence of OVA (10 μg/ml) or medium only. As a positive control, cells are cultured in the presence of plate-bound rat anti-mouse CD3 (clone 17A2, 50 μg/ml, coated overnight at 4° C.). Each in vitro stimulation is performed in triplicate. After 5 days of culture at 37° C., the supernatant is harvested, pooled per stimulation, and stored at −20° C. until cytokine levels were determined by ELISA.

The IFN-α, IL-4, IL-5, IL-10, and IL-13 ELISAs are performed according to the manufacturer's instructions (PharMingen, San Diego, Calif.). The detection limits of the ELISAs are 160 pg/ml for IFN-γ, 16 pg/ml for IL-4, 32 pg/ml for IL-5, and 100 pg/ml for IL-10 and IL-13.

In all experiments, airway responsiveness to methacholine, OVA-specific IgE levels in serum, cellular infiltration in the BALF, and T-cell responses in lung tissue are measured 24 hours after the last challenge in each mouse.

Improvements in asthma in the experimental mice are marked by a decrease in the number of mononuclear cells (including monocytes, macrophages, and lymphocytes), eosinophils, and neutrophils in the BALF, a decrease in the airway hyperresponsiveness, and a decrease in the cytokine production by antigen-restimulated T cells in the lung tissue.

### Example 14

TNFα Inhibitor in Mouse Model of Chronic Ostructive Pulmonary Disease (COPD) (Study Examining Treatment for Alveolar Enlargement and Inflammation)

The following study is performed using a cigarette smoke induced COPD mouse model (Keast, D. et al. (1981) J. Pathol. 135:249; Hautmaki, R. D., et al. (1997) Science 277:2002, both entirely incorporated by reference). In response to cigarette smoke, inflammatory cell recruitment into the lungs followed by pathologic changes characteristic of emphysema have been observed.

Mice are exposed to smoke from two non-filtered cigarettes per day, 6 days per week, for 6 months, with the use of a smoking apparatus with the chamber adapted for mice. Nonsmoking, age-matched animals are used as controls. After 6 months of exposure to smoke as described above, XPro™ 1595 is administered in a range of doses according to standard protocols known in the art. An appropriate placebo control is also administered. Mice are administered the antibody treatment for a period of 21 days. Mice are sacrificed, followed by examination of lung volume and compliance, cytokine measurement, histological mucus index measurement, alveolar duct enlargement, air space measurement, alveolar and interstitial macrophage counts and alveolar size, as described below.

Following XPro™ 1595 treatment, bronchiolar lavage is performed on euthanized animals; the trachea is isolated by blunt dissection, and small caliber tubing is inserted and secured in the airway. Two volumes of 1.0 ml of PBS with 0.1% BSA are instilled, gently aspirated, and pooled. Each BAL fluid sample is centrifuged, and the supernatants are stored in −70° until used. Cytokine measurements are as described in above Example.

To determine lung volume and compliance, animals are anesthetized, the trachea is cannulated, and the lungs are ventilated with 100% O₂ via a “T” piece attachment. The trachea is then clamped and oxygen absorbed in the face of ongoing pulmonary perfusion. At the end of this degassing, the lungs and heart are removed en bloc and inflated with PBS at gradually increasing pressures from 0 to 30 cm. The size of the lung at each 5-cm interval is evaluated via volume displacement. An increase in the lung volume of treated animals compared to placebo treated control animals indicates an improvement in COPD.

For histological analysis, animals are sacrificed and a median sternotomy is performed, and right heart perfusion is accomplished with calcium- and magnesium-free PBS to clear the pulmonary intravascular space. The lungs are then fixed to pressure (25 cm) with neutral buffered 10% formalin, fixed overnight in 10% formaliii, embedded in paraffin, sectioned at 5 μm and stained with Hematoxylin and eosin (H&E) and periodic acid-Schiff with diastase (D-PAS).

The histological mucus index (HMI) provides a measurement of the percentage of epithelial cells that are D-PAS+per unit airway basement membrane. It is calculated from D-PAS-stained sections (Cohn, L., et al. (1997) J. Exp. Med. 186:1737). A decrease in the HMI of treated animals compared to placebo treated control animals indicates an improvement in COPD.

Lm, an indicator of air space size, is calculated for each mouse from 15 random fields at x200 by means of a 50-line counting grid (10-mm total length). The results are the average of measurements of two independent investigators. An increase in air space size of treated animals compared to placebo treated control animals indicates an improvement in COPD.

To determine alveolar duct enlargement, the proximal surface areas from the terminal bronchiole-alveolar duct transition extending 250 pm into the duct using Optimus 5.2 image analysis software (Optimus, Bothell, Wash.) is measured. A decrease in alveolar duct size of treated animals compared to placebo treated control animals indicates an improvement in COPD.

Alveolar and interstitial macrophages are quantitated by counting macrophages identified by murine Mac-3 (rat antibody to mouse (0.5 mg/ml), used at 1:4000 dilution; PharMingen, San Diego, Calif.O immunostaining using the avidin-biotin alkaline. A decrease in the number of alveolar and interstitial macrophages of treated animals compared to placebo treated control animals indicates an improvement in COPD.

Alveolar size is estimated from the mean cord length of the airspace (Ray, P., et al. (1997) J. Clin. Invest. 100:2501). This measurement is similar to the mean linear intercept, a standard measure of air space size, but has the advantage that it is independent of alveolar septa! thickness. Sections are prepared as described above. To obtain images at random for analysis, each glass slide is placed on a printed rectangular grid and a series of dots placed on the coverslip at the intersection of the grid lines, i.e., at intervals of approximately 1 mm. Fields as close as possible to each dot are acquired by systematically scanning at 2-mm intervals. Fields containing identifiable artifacts or non-alveolated structures such as bronchovascular bundles or pleura are discarded.

A minimum of ten fields from each mouse lung are acquired into a Macintosh G3 computer (Apple Computer Inc., Cupertino, Calif., USA) through a framegrabber board. Images are acquired in 8-bit gray-scale at a final magnification of 1.5 pixels per micron. The images are analyzed on a Macintosh computer using the public domain NIH Image program written by Wayne Rasband at NIH using a custom-written macro available from the web site (http://rsb.info.nih.gov/nih-image). Images are manually thresholded and then smoothed and inverted. The image is then subject to sequential logical image match “and” operations with a horizontal and then vertical grid. At least 300 measurements per field are made for each animal. The overlying air space air is averaged as the mean chord length. Chord length increases with alveolar enlargement. An increase in alveolar size of treated animals compared to placebo treated control animals indicates an improvement in COPD.

### Example 15

TNFα Inhibitor in Idiopathic Pulmonary Fibrosis (IPF) Mouse Model (Study of IPF Treatment using Bleomycin Induced Lung Fibrosis Mouse Model)

The following study is performed using the bleomycin induced lung fibrosis mouse model (reviewed in Bowden, D. H. (1984) Lab. Invest. 50:487; Tokuda, A., et al. (2000) J. Immunol. 164:2745).

Bleomycin sulfate is administered to C57BL/6J female mice aged 8-10 weeks. Briefly, C57BL/6J mice are anesthetized with 200 μl of 5 mg/ml pentobarbital injected i.p., followed by intratracheal instillation of 3 mg/kg bleomycin sulfate in 50 μl sterile saline.

XPro™ 1595 is administered to the bleomycin induced lung fibrosis mice in a range of doses, after intratracheal instillation of bleomycin as described above. An appropriate placebo control is also administered. Mice are treated twice daily for 14 days.

B5] Mice are sacrificed 20 and 60 days following bleomycin treatment. Tissues are fixed in 10% buffered formalin and embedded in paraffin. Sections are stained with hematoxylin and eosin and examined by light microscopy. Lung-infiltrating leukocyte counts, cytokine measurements, and total lung collagen content is determined as described below.

BAL cells and lung-infiltrating leukocytes are prepared as described in Smith et al. (1994) J. Immunol. 153:4704. In brief, following anesthesia, 1 ml PBS is instilled and withdrawn five times from the lung via an intratracheal cannula. The BAL fluids are collected and after RBC lysis total leukocyte counts are determined. Cell differentials are determined after cytospin centrifuge. Specimens are stained with Diff-Quik products (Baxter, Miami, Fla.).

To isolate lung-infiltrating leukocytes, lungs are perfused with saline, dissected from the chest cavity, and then minced with scissors. Each sample is incubated for 30 minutes at 37° C. on a rocker in 15 ml digesting buffer (10% FCS in RPM! 1640 with 1% collagenase; Wako Pure Chemical, Osaka, Japan). Next, the sample is pressed through nylon mesh and suspended in 10% FCS-RPMI 1640 after being rinsed. The cell suspension is treated with Histopaque-1119 (Sigma, St. Louis, Mo.) and centrifuged at 2000 rpm for 20 min to remove lung parenchymal cells and RBC. The pellet is resuspended in 2.5% FCS-PBS after being rinsed. After cell counts are performed, flow cytometric immunofluorescence analyses are conducted.

Immunofluorescence analyses of peripheral blood leukocytes and lung-infiltrating leukocytes are performed with the use of an Epics Elite cell sorter (Coulter Electronics, Hialeah, Fla.) as described previously (Yoneyama et al. (1998) J. Clin. Invest. 102:1933; Murai et al. (1999) J. Clin. Invest. 1041:49, both entirely incorporated by reference). Peripheral blood leukocytes are prepared from normal mice with RBC lysis buffer. After incubation with Fc Block (anti-CD16/32; Pharmingen, San Diego, Calif.) for 10 min, cells are stained with PE-conjugated mAb against CD3, CD4, CD8, CD11 b, CD11c, and Gr-1 (Pharmingen), and also stained with 20 μg/ml of rabbit anti-CCR1 polyclonal Ab followed by staining with FITC-conjugated goat anti-rabbit IgG (Leinco Technologies. St. Louis, Mo.). Before analyses propidium iodide (Sigma) staining is performed to remove the dead cells. A decrease in the number of lung-infiltrating leukocytes of treated animals compared to placebo treated control animals indicates an improvement in IPF.

Immunohistochennistry of lung samples is carried out as follows: lung specimens are prepared as described previously (Yoneyama et al. (1998) J. Clin. Invest. 102:1933; Murai et al. (1999) J. Clin. Invest. 104:49, both entirely incorporated by reference). Lung specimens are fixed in periodate-lysineparaformaldehyde, washed with PBS containing sucrose, embedded in Tissue-Tek OCT compound (Miles, Elkhart, Ind.), frozen in liquid nitrogen, and cut into 7-pm-thick sections with a cryostat. After inhibition of endogenous peroxidase activity, the sections are incubated with the first Ab. The Abs used are rabbit anti-CCR1 Ab, rat anti-F4/80 (BMA Biomedicals, Geneva, Switzerland), rat anti-CD4, rat anti-CD8, rat anti-Gr-1 (Pharmingen), rat anti-nonlymphoid dendritic cell (NLDC)-145, and rat antiMHC class II (BMA Biomedicals). As a negative control either a rabbit IgG or a rat IgG is used, respectively. They are treated sequentially with either HRP-conjugated goat anti-rabbit IgG (Cedarlane Laboratories, Hornby, Ontario, Canada) or a HRP-conjugated goat anti-rat IgG (BioSource International, Camarillo, Calif.). After staining with 3,3′-diaminobenzidine (Wako Pure Chemical) or 3-amino-9-ethylcarbazole substrate kit (Vector Laboratories, Burlingame, Calif.), samples are counterstained with Mayer's hematoxylin. A decrease in CCR1, and decreases in the number of CD4+ T cells, CD8+ T cells, nonlymphoid dendritic cell (NLDC), and MHC class II bearing cells of treated animals compared to placebo treated control animals indicates an improvement in IPF.

Total lung collagen content is determined by assaying total soluble collagen using the Sircol Collagen Assay kit (Biocolor, Northern Ireland) according to the manufacturer's instructions. Briefly, lungs are harvested at day 14 after bleomycin administration and homogenized in 10 ml 0.5 M acetic acid containing about 1 mg pepsin/10 mg tissue residue. Each sample is incubated for 24 h at 4° C. with stirring. After centrifugation, 200 μl of each supernatant is assayed. One milliliter of Sircol dye reagent that binds to collagen is added to each sample and then mixed for 30 min. After centrifugation, the pellet is suspended in 1 ml of the alkali reagent included in the kit and read at 540 nm by a spectrophotometer. Collagen standard solutions are utilized to construct a standard curve. Collagens contain about 14% hydroxyproline by weight, and collagen contents obtained with this method correlate well with the hydroxyproline content according to the manufacturer's data. A decrease on lung collagen content of treated animals compared to placebo treated control animals indicates an improvement in IPF.

Using the bleomycin induced lung fibrosis mouse model, mice are examined for a decrease in the BAL cell counts, a decrease in the peripheral blood leukocytes and lung infiltrating leukocytes. Mice are also examined for a decrease in the total lung collagen content in XPro™ 1595 treated mice as compared to placebo treated mice.

### Example 16

### TNFa Inhibitor in Treatment of Asthma

Clinical Study of XPro™ 595 in Human Subjects with Asthma

Patients 12 to 65 years of age are eligible for the study if they have had a documented diagnosis of asthma of at least 2 years duration and have also had demonstrable reversible bronchospasm with an increase in FEV1 of 15% or greater after the administration of albuterol within the previous six months. Additional inclusion criteria include, a baseline FEV I between 50% and 80% of predicted normal, absence of any clinically significant disease other than asthma, a history of daily use of inhaled corticosteroids and cessation of all p32-agonist use 30 days prior to the beginning of the study.

A baseline visit occurs within 7 days after the screening visit. All patients undergo evaluation of FEV1 and have a complete physical examination. Pulmonary auscultation and oropharyngeal examinations are performed, and asthma symptoms are assesses. Patients who qualify are randomly assigned to a treatment group including a placebo group.

Following baseline measurements, patients begin receiving treatment. They are randomized and treated with either XPro™ 1595 or placebo in a blinded fashion. At days 15 and 29, all examinations performed at the baseline visit are repeated. A 12-lead ECG is also performed. Diary cards are reviewed with patients regarding the use of other medications and any adverse events.

Improvements are determined on spirometry tests measured at each visit. These include FEV1, peak expiratory flow rate (PEFR), Forced Vital Capacity (FCV), and forced expiratory flow at 25% to 75% of FVC. FEV1 at the final visit is regarded as the primary measure of efficacy. Twice-daily PEFR tests performed by the patient are compared and the number of inhalations of rescue medication is calculated. Patient/physician evaluations of asthma symptoms (wheezing, tightness in the chest, shortness of breath and cough) are characterized by severity. Compliance is assessed by review of the patient's diary cards and by collecting unused study medication.

### Example 17

TNFα Inhibitor in Treatment of COPD (Clinical Study Examining XPro™ 1595 in Human Subjects with COPD)

The study population is male and female subjects who are 40 to 80 years of age with a diagnosis of COPD. Subjects must have a best FEV1/FVC ratio ˆ0.70 liters, fixed airway obstruction, defined by □15% or □200 ml (or both) increase in FEV1 after the administration of albuterol and a post-albuterol FEV1 between 30 and 70% of predicted. Subjects must also be current or previous smokers with a history of smoking □10 pack years.

Following baseline measurements, patients begin receiving treatment. They are randomized and treated with either XPro™ 1595 or placebo in a blinded fashion.

Improvements are marked by an increase from predose baseline after study medication in pre-bronchodilator FEV1 and change from baseline in total score of the St. George's Respiratory Questionnaire (Jones, P. W., et al. (1991) Resp. Med. 85(suppl):25) which indicates an improvement in the patients' quality of life. Improvements are also seen as an increase from baseline FVC at trough, an increase in time to first COPD exacerbation, and a decrease from baseline in post-exercise breathlessness (modified Borg Scale; Stulbarg, M., Adams, L. Dyspnea. In: Murray J, Nadel J, eds. Textbook of Respiratory Medicine. Philadelphia, Pa.: W B Saunders, 2000; 541-552). Measures of safety are adverse events, vital signs, electrocardiogram at all double-blind visits, and laboratory assessments.

### Example 18

TNFα Inhibitor in Treatment of IPF (Clinical Study of XPro™ 1595 in Human Subjects with IPF.

A multi-center, double-blind, placebo-controlled study comparing treatment of IPF patients with XPro™ 1595 versus treatment with placebo is performed. Patients are eligible for the study if they have histologically verified IPF and have a decline in lung function of at least 10% during the 12 months prior to the beginning of the study, despite continuous or repeated treatment with glucocorticoids or other immunosuppressive agents or both for at least 6 months. The main histological feature used to identify IPF is the presence of subpleural and periacinar fibrotic lesions with only minor cellular infiltration. The absence of bilateral patchy infiltrates on high-resolution computed tomography and the demonstration of predominantly peripheral distribution of lesions are the radiological criteria for identifying the disease. Patients with a history of exposure to organic or inorganic dust or drugs known to cause pulmonary fibrosis and those with connective-tissue disease or other chronic lung diseases are excluded. Patients with end-stage IPF as identified on the basis of a total lung capacity of less than 45% of the predicted normal are also excluded. Baseline values for repeat pulmonary function tests, FVC, total lung capacity (TLC), and oxygen saturation are taken.

Following baseline measurements, patients begin receiving treatment. They are randomized and treated with either XPro™ 1595 or placebo in a blinded fashion.

Improvements in IPF patients include an increase in the overall survival rate of patients in the study, and improvements in FVC, total lung capacity (TLC) and oxygen saturation. Improvement in pulmonary function is defined as a 10% or greater increase in predicted value of FVC or TLC, or a 3% or greater increase in oxygen saturation with the same fraction of expired air, resting or exertional. A decrease of similar manner for each measure is considered a deterioration. Patients who do not demonstrate improvement or deterioration are considered stable.

Whereas particular embodiments of the invention have been described above for purposes of illustration, it will be appreciated by those skilled in the art that numerous variations of the details may be made without departing from the invention as described in the appended claims. All references described above are herein incorporated by reference.

We claim:

1. A composition for treating a TNF-α related disorder, said composition comprising a therapeutic agent and a variant human TNF-α homotrimer.

2. The composition of claim 1, wherein each monomer of said human TNF-α is a non-naturally occurring variant TNF-α as compared to human wild-type TNF-α (SEQ ID NO:1) comprising a substitution at a position selected from the group consisting of positions 21, 23, 30, 31, 32, 33, 34, 35, 57, 65, 66, 67, 69, 75, 84, 86, 87, 91, 97, 101, 111, 112, 115, 140, 143, 144, 145, 146 and 147.

3. The composition according to claim 2, wherein said variant TNF-α protein has from 2 to 5 amino acid substitutions as compared to SEQ ID NO:1.

4. The composition of claim 2, wherein said substitution is at a position selected from the group consisting of positions 31, 57, 69, 75, 86, 87, 97, 101, 115, 143, 145, and 146.

5. The composition of claim 1, wherein each monomer of said TNF-α homotrimer comprises an amino acid sequence that has at least one amino acid substitution in the Large Domain and at least one amino acid substitution in a domain selected from the group consisting of the DE Loop and the Small Domain as compared to SEQ ID NO:1, wherein said Large Domain substitution is at a position selected from the group consisting of 21, 30, 31, 32, 33, 35, 65, 66, 67, 111, 112, 115, 140, 143, 144, 145 and 146, said Small Domain substitution at a position selected from the group consisting of 75 and 97, said DE Loop substitution at a position selected from the group consisting of 84, 86, 87 and 91, and said monomers are capable of forming TNF-α heterotrimers having at least a 50% decrease in receptor activation as compared to a homotrimer of wild-type TNF-α proteins as determined by a caspase assay.

6. The composition of claim 2, wherein said substitutions are selected from the group consisting of Q21C, Q21R, E23C, N34E, V91E, Q21R, N30D, R31C, R311, R31D, R31E, R32D, R32E, R32S, A33E, N34E, N34V, A35S, D45C, L57F, L57W, L57Y, K65D, K65E, K651, K65M, K65N, K65Q, K65T, K65S, K65V, K65W, G66K, G66Q, Q67D, Q67K, Q67R, Q67S, Q67W, Q67Y, C69V, L75E, L75K, L75Q, A84V, S86Q, S86R, Y87H, Y87R, V91E, 197R, 197T, C101A, A111R, A111E, K112D, K112E, Y115D, Y115E, Y115F, Y115H, Y1151, Y115K, Y115L, Y115M, Y115N, Y115Q, Y115R, Y115S, Y115T, Y115W, D140K, D140R, D143E, D143K, D143L, D143R, D143N, D143Q, D143R, D143S, F144N, A145D, A145E, A145F, A145H, A145K, A145M, A145N, A145Q, A145R, A145S, A145T, A145Y, E146K, E146L, E146M, E146N, E146R, E146S and S147R.

7. The composition of claim 6, wherein said substitution is selected from the group consisting of R31 C, C69V, Y87H, C101A, and A145R.

8. The composition of claim 7, wherein said monomer comprises the substitutions VIM, R31 C, C69V, Y87H, C101A, and A145R.

9. The composition of claim 1, wherein said therapeutic agent is an anti-rheumatoid arthritis agent selected from the group consisting of a non-steroidal anti-inflammation drugs (NSAID), a disease-modifying antip rheumatic drugs (DMARD), and a steroid.

10. The composition of claim 1, wherein the therapeutic agent is an anti-psoriasis agent selected from the group consisting of anthralin, chrysarobin, corticosteroids, calcipotriene, vitamin D, TazaroteneVitamin A derivatives, methotrexate, cyclosporine, acitretin, alefacept, etanercept, and efalizumab.

11. The composition of claim 1, wherein the therapeutic agent is an anti-inflammatory medication.

12. A method of treating a TNF-α associated disorder comprising administering an effective amount of the composition according to claim 1 to a patient in need thereof.


SEQUENCE LISTING (23 sequences)

SEQ ID NO: 1 - protein, 164 aa, Homo sapiens
MHHHHHHVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 2 - DNA, 495 bp, Homo sapiens
atgcaccaccaccaccaccacgtacgctcctcctcccgcactccgtccgacaaaccggtagctcacgtagtagctaacccgcaggctgaaggtcagctgcagtggctgaaccgccgcgctaacgctctgctggctaacggtgtaaaactgcgcgacaaccagctggtagtaccgtccgaaggtctgtacctgatctactcccaggtactgttcaaaggtcagggttgtccgtccactcacgtactgctgactcacactatctcccgcatcgctgtatcctaccagactaaagtaaacctgctgtccgctatcaaatccccgtgtcagcgcgaaactccggaaggtgctgaagctaaaccgtggtacgaaccgatctacctgggtggtgtattccagctggaaaaaggtgaccgcctgtccgctgaaatcaaccgcccgaactacctggacttcgctgaatccggtcaggtatacttcggtatcatcgctctgtga

SEQ ID NO: 3 - protein, 47 aa, Homo sapiens
TRAFDQDKIEALSSKVQQLERSIGLKDLAMADLEQKVLEMEASTYDG

SEQ ID NO: 4 - protein, 47 aa, Homo sapiens
TRAFVARNTGLLESQLSRHDQMLSVHDIRLADMDLRFQVLETASYNG

SEQ ID NO: 5 - protein, 47 aa, Homo sapiens
TRAFNDQRLAVLEEETNKHDTHINIHKAQLSKNEERFKLLEGTCYNG

SEQ ID NO: 6 - protein, 47 aa, Homo sapiens
TRAFDRERILSLEQRVVELQQTLAQKDQALGKLEQSLRLMEEASFDG

SEQ ID NO: 7 - protein, 47 aa, Homo sapiens
TRAFQDHQIRELTAKMETQSMYVSELKRTIRTLEDKVAEIEAQQCNG

SEQ ID NO: 8 - protein, 31 aa, Homo sapiens
TRAFCALVSRQRQELQELRRELEELSVGSDG

SEQ ID NO: 9 - protein, 8 aa, Homo sapiens
HHHHHHVR

SEQ ID NO: 10 - protein, 10 aa, Homo sapiens
SSRTPSDKPV

SEQ ID NO: 11 - protein, 6 aa, Homo sapiens
LNRRAN

SEQ ID NO: 12 - protein, 11 aa, Homo sapiens
NGVELRDNQLV

SEQ ID NO: 13 - protein, 7 aa, Homo sapiens
PSEGLYL

SEQ ID NO: 14 - protein, 5 aa, Homo sapiens
LFKGQ

SEQ ID NO: 15 - protein, 8 aa, Homo sapiens
CPSTHVLL

SEQ ID NO: 16 - protein, 5 aa, Homo sapiens
VSYQT

SEQ ID NO: 17 - protein, 5 aa, Homo sapiens
SAIKS

SEQ ID NO: 18 - protein, 12 aa, Homo sapiens
AEAKPWYEPIYL

SEQ ID NO: 19 - protein, 11 aa, Homo sapiens
GVFQLEKGDRL

SEQ ID NO: 20 - protein, 6 aa, Homo sapiens
GSGGSN

SEQ ID NO: 21 - protein, 6 aa, Homo sapiens
GGGGSN

SEQ ID NO: 22 - protein, 5 aa, Homo sapiens
GGGSN

SEQ ID NO: 23 - protein, 156 aa, Homo sapiens
VRSSSRTPSDKPVAHVVANPQAEGQLQWLNRRANALLANGVELRDNQLWPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL